(19) United States
(12) Patent Application Publication
(10) Pub. No.: US 2012/0121656 A1
(43) Pub. Date: May 17, 2012

(54) METHODS AND COMPOSITIONS FOR PROTECTING AGAINST NEUROTOXICITY OF A NEUROTOXIC AGENT, AND IMPROVING MOTOR COORDINATION ASSOCIATED WITH A NEURODEGENERATIVE CONDITION OR DISEASE

(75) Inventors: Richard L. Watson, McPherson, KS (US); Anthony B. Wood, Dallas, TX (US); Gregory J. Archambeau, Puyallup, WA (US)

(73) Assignee: Revalesio Corporation, Tacoma, WA (US)

(21) Appl. No.: 13/097,565

(22) Filed: Apr. 29, 2011

Related U.S. Application Data
(60) Provisional application No. 61/413,899, filed on Nov. 15, 2010.
(60) Provisional application No. 61/454,409, filed on Mar. 18, 2011.

(51) Int. Cl. A61K 33/00; A61K 31/785; A61K 38/21; A61P 29/00; A61P 25/28; A61P 25/16; A61P 39/00; A61K 9/00; A61K 39/395; B82Y 5/00

(52) U.S. Cl. 424/400; 424/613; 424/78.37; 424/85.6; 424/133.1; 424/172.1; 977/915

(57) ABSTRACT

Provided are methods for protecting against or reducing neurotoxicity of exposure to a neurotoxic agent, comprising administering an electrokinetically altered aqueous fluid as provided herein in an amount sufficient to provide for neuroprotection against the neurotoxic agent, preferably where protecting against or reducing loss of motor coordination in the subject exposed to the neurotoxin is afforded. In certain aspects, protecting or reducing neurotoxin-mediated neuronal apoptosis is afforded, and/or activating or inducing at least one of PI-3 kinase and Akt phosphorylation in neurons is afforded. Preferably, administering the fluid comprises administering the fluid prior to exposure to the neurotoxic agent. Additionally provided are methods for preserving or improving motor coordination in a subject having a neurodegenerative condition or disease, comprising administering an electrokinetically altered aqueous fluid as provided herein in an amount sufficient to provide for preserving or improving motor coordination in the subject.

### CROSS-REFERENCE TO RELATED APPLICATIONS

The Application claims the benefit of priority to U.S. Provisional Patent Application Nos. 61/413,899 filed 15 Nov. 2010 and entitled “METHODS AND COMPOSITIONS FOR PROTECTING AGAINST NEUROTOXICITY OF A NEUROTOXIC AGENT, AND IMPROVING MOTOR COORDINATION ASSOCIATED WITH A NEURODEGENERATIVE CONDITION OR DISEASE,” and 61/454,409 filed 18 Mar. 2011 of same title, both of which are incorporated herein by reference in their entirety.

### FIELD OF THE INVENTION

Particular aspects relate generally to methods for protecting against or reducing neurotoxicity of exposure to a neurotoxic agent, comprising administering an electrokinetically altered aqueous fluid as provided herein, and preferably wherein protecting against or reducing loss of motor coordination in the subject exposed to the neurotoxin is afforded. Particular aspects relate to protecting or reducing neurotoxin-mediated neuronal apoptosis and/or activating or inducing at least one of PI-3 kinase and Akt phosphorylation in neurons. Particular aspects relate generally to methods for preserving or improving motor coordination in a subject having a neurodegenerative condition or disease, comprising administering an electrokinetically altered aqueous fluid as provided herein.

### BACKGROUND OF THE INVENTION

Neurodegenerative diseases are a group of diseases typified by deterioration of neurons or their myelin sheath. This destruction of neurons eventually leads to dysfunction and disabilities. Often times inflammation is found to be a component of neurodegenerative diseases and adds to the pathogenesis of the neurodegeneration (Minagar, et al. (2002) J. Neurological Sci. 202:13-23; Antel and Owens (1999) J. Neuroimmunol. 100: 181-189; Elliott (2001) Mol. Brain. Res. 95:172-178; Nakamura (2002) Biol. Pharm. Bull. 25:945-953; Whitton PS. (2007) Br J Pharmacol. 150:963-76). Collectively, these diseases comprise the art-recognized inflammatory neurodegenerative diseases. Neuroinflammation may occur years prior to any considerable loss of neurons in some neurodegenerative disorders (Tansey et. al., Fron Bioscience 13:709-717, 2008). Many different types of immune cells, including macrophages, neutrophils, T cells, astrocytes, and microglia, can contributed to the pathology of immune-related diseases, like Multiple Sclerosis (M.S.), Parkinson's disease, amyloidosis (e.g., Alzheimer's disease), amyotrophic lateral sclerosis (ALS), prion diseases, and HIV-associated dementia. More specifically, research groups have noted that in MS the injury to myelin is mediated by an inflammatory response (Ruffini et. al. (2004) Am J Pathol 164:1519-1522) and that M.S. pathogensis is exacerbated when leukocytes infiltrate the CNS (Dos Santos et. al. (2008) J Neuroinflammation 5:49). One research group has developed genetic models to test

CNS inflammation and its effects in MS (through the animal model experimental autoimmune encephalomyelitis (EAE). In addition, pro-inflammatory cytokines (specifically TNF-alpha) were found to be elevated in Alzheimer's disease, Parkinson's disease, and amyotrophic lateral sclerosis (ALS). (Greig et al (2006) Ann NY Acad of Sci 1035:290-315). These inflammatory neurodegenerative diseases may, therefore, be effectively treated by anti-inflammatory drugs.

Inflammatory neurodegenerative diseases include but are not limited to: multiple sclerosis (MS), Parkinson's disease, amyloidosis (e.g., Alzheimer's disease), amyotrophic lateral sclerosis (ALS), HIV-associated dementia, stroke/cerebral ischemia, head trauma, spinal cord injury, Huntington's disease, migraine, cerebral amyloid angiopathy, AIDS, age-related cognitive decline; mild cognitive impairment and prion diseases in a mammal.

Multiple sclerosis (MS) is a chronic inflammatory neurodegenerative disease of the central nervous system (CNS) that affects approximately 1,100,000 people all over the world, in particular affects young adults (Pugliatti et al. (2002) Clin. Neurol. Neuros. 104:182-191). MS is characterized pathologically by demyelination of neural tissue, which results clinically in one of many forms of the disease, ranging from benign to chronic-progressive patterns of the disease state. More specifically, five main forms of multiple sclerosis have been described: 1) benign multiple sclerosis; 2) relapsing-remitting multiple sclerosis (RRMS); 3) secondary progressive multiple sclerosis (SPMS); 4) primary progressive multiple sclerosis (PPMS); and 5) progressive-relapsing multiple sclerosis (PRMS). Chronic progressive multiple sclerosis is a term used to collectively refer to SPMS, PPMS, and PRMS. The relapsing forms of multiple sclerosis are SPMS with superimposed relapses, RRMS and PRMS.

Throughout the course of the disease there is a progressive destruction of the myelin sheath surrounding axons. Since intact myelin is essential in the preservation of axonal integrity (Dubois-Dalcq et al., Neuron. 48, 9-12 (2005)) systematic destruction eventually leads, clinically, to various neurological dysfunctions including numbness and pain, problems with coordination and balance, blindness, and general cognitive impairment. Interestingly, MS progression can differ considerably in patients with some having slight disability even after several decades of living with the disease, while others becoming dependent upon a wheelchair only a few years after being diagnosis.

The etiology of MS currently is unknown, but studies examining genetic evidence, the molecular basis, and immunology factors are beginning to elucidate the course of the disease and the mechanism by which demylination occurs. In genetic analyses, some reports have indicated that related individuals have higher incidence of MS when compared to normal population (0.1% prevalence of MS): an identical twin having a 30% chance of developing the disease if the other twin has MS and fraternal twins and siblings have a 1-2% chance if a another sibling is affected by MS. Several groups have utilized linkage and association studies to discover the genes responsible for this heritability and found that the relative risk of being affected by MS is 3-4 fold higher to those carrying a the major histocompatibility complex (MHC) class II allele of the human leukocyte antigen (HLA)-DR2 allele. Other genes have been identified that associate with MS, but a much lower risk. The link between MS susceptibility and MHC Class II strongly suggests a role for CD4+T-cells in the pathogenesis of MS (Oksenberg et al., JAMA 270:2363-2369 (1993); Olerup et al., Tissue Antigens 38:1-3 (1991)).

In addition, identification of genes that are differentially expressed in MS patients suffering from MS compared to healthy individuals has been attempted. Gene microarrays have been used 1) to examine transcription from MS plaque types (acute verses chronic) and plaque regions (active verses inactive) (Lock and Heller (2003)); 2) to compare peripheral blood mononucleocytes (PBMC) in RRMS patients verses controls, from patients both with and without interferon-β treatment (Sturzebecher et al. (2003)); and 3) to examine CNS cells in stages of experimental allergic encephalomyelitis (EAE) in mice, an animal model of MS (Lock et al. (2002)). Much of what these experiments discovered was expected, including the finding that anti-inflammatory, anti-apoptotic genes are down-regulated and pro-inflammatory, proliferation genes are up-regulated. Surprising results include identification of potential novel targets for therapeutic application such as osteopontin (Chabas et al. 2001) and TRAIL (Wandinger et al. 2003)). However, many of the genes that have differential regulation when comparing expression from MS patients with healthy individuals have unknown significance in MS development, because any genes that may affect MS susceptibility and/or progression are still unknown.

Further research has determined that inflammatory responses initiated by autoreactive CD4+ T-cells can mediate injury to myelin (Bruck et al., J. Neurol. Sci. 206:181-185 (2003)). In general, it is believed that much of the damage occurring to myelin sheaths and axons during an episode of MS happens through autoreactive T cell response which produces an inflammatory response including the secretion of proinflammatory (e.g. Th1 and Th17) cytokines (Prat et al., J. Rehabil. Res. Dev. 39:187-199 (2002); Hemmer et al., Nat. Rev. Neurosci. 3:291-301 (2002)).

Treatments that currently are available for MS include glatiramer acetate, interferon-β, natalizumab, and mitoxanthrone. In general, these drugs suppress the immune system in a nonspecific fashion and only marginally limit the overall progression of disease. (Lubetzki et al. (2005), Curr. Opin. Neurol. 18:237-244). Thus, there exists a need for developing therapeutic strategies to better treat MS.

Glatiramer acetate is composed of glutamic acid, lysine, alanine, and tyrosine as a random polymer. Glatiramer acetate has limited effectiveness and significant side effects, for example, lump at the site of injection, chills, fever, aches, shortness of breath, rapid heartbeat and anxiety. In an important clinical study using 943 patients with primary progressive MS, glatiramer acetate failed to halt the progression of disability and the disease (Wolinsky, et al (2007) Ann Neurol 61:13-24).

Interferon-β is a naturally occurring protein produced by fibroblasts and part of the innate immune response. As a drug for MS, interferon-β is about 18-38% effective in reducing the rate of MS episodes. Side effects include mild ones flu-like symptoms and reactions at the site of injection and more serious (e.g., depression, seizures, and liver problems)

Mitoxantrone is a treatment for MS. It was developed as a chemotherapy treatment for use in combating cancer—working by interfering with DNA repair and synthesis and is not specific to cancer cells. Side effects from mitoxantrone can be quite severe and include nausea, vomiting, hair loss, heart damage, and immunosuppression.

Natalizumab is a humanized monoclonal antibody that targets alpha4-integren, which is a cellular adhesion molecule. Natalizumab is believed to work by keeping immune cells that cause inflammation from crossing the blood brain barrier (BBB). Side effects include fatigue, headache, nausea, colds, and allergic reactions.

### Parkinson's Disease

Parkinson's disease (PD), another inflammatory neurodegeneration disease, is characterized by movement disorders, including muscle rigidity and slow physical movements. PD is the second most frequent neurodegenerative disorder, affecting up to 1 million people in the US alone1. PD prevalence increases with age, from 0.3% in the general US population to 1% to 2% in persons aged 65 years or older, and 4% to 5% in individuals aged 85 years or older1.

With an overall increasing life expectancy, numbers of PD patients in the US and other countries are expected to double by 20302.

PD is a progressive disease characterized by motor symptoms that include tremor, rigidity, bradykinesia (slowness of movement), gait impairment, and postural change. The disease also involves non-motor symptoms such as cognitive deficits, depression, and sleep disorders. Like Alzheimer's disease, PD is a proteinopathy. Misfolded α-synuclein accumulates inside neurons and forms so-called Lewy bodies, one of the neuropathological hallmarks of PD. Initially thought to be caused exclusively by the loss of dopaminergic neurons in the substantia nigra, PD recently has been recognized to have an inflammatory component that activates brain microglial cells and is involved in the progression of neuronal cell death. A perceived pathophysiological cause of Parkinson's disease is progressive destruction of dopamine producing cells in the basal ganglia which comprise the pars compartum of the substantia nigra, basal nuclei located in the brain stern. Loss of dopamineric neurons results in a relative excess of acetylcholine. Jellinger, K. A., Post Mortem Studies in Parkinson's Disease—Is It Possible to Detect Brain Areas For Specific Symptoms?, J Neural Transm 56 (Supp); 1-29:1999. In addition, recent research into Parkinson's disease has observed that due to enhanced expression of cytokines and HLA-DR antigens it is likely that the immune response contributes to the neuronal damage (Czlonkowska et. al. (2002) Med Sci Monit 8:RA165-77).

Effective treatment at an early stage represents an unmet clinical need in the care of PD patients. Levodopa (L-DOPA) is the most efficacious pharmacologic treatment for PD, but is usually prescribed late in the course of the disease due to severe side effects. Dopamine receptor agonists and monoamine oxidase type B inhibitors have shown an inverse correlation between efficacy and the occurrence and severity of side effects, and trials exploring other treatment options including coenzyme Q10, tocopherol (Vitamin E), amantidine, and beta-blockers have either failed to demonstrate benefits or have not produced sufficient data for a thorough risk vs. benefit evaluation. Neuroprotection in particular has been a key, yet elusive, goal in PD treatment.

Amyloidosis develops when certain proteins have altered structure and tend to bind to each building up in particular tissue and blocking the normal tissue functioning. These altered structured proteins are called amyloids. Often amyloidoses is split into two categories: primary or secondary. Primary amyloidoses occur from an illness with improper immune cell function. Secondary amyloidoses usually arise from a complication of some other chronic infectious or inflammatory diseases. Examples of such include Alzheimer's disease and rheumatoid arthritis. Since the underlying problem in secondary amyloidosis is inflammation, treating inflammation likely will be beneficial.

Alzheimer's disease is another type of inflammatory neurodegenerative disease. It is exemplified by the increasing impairment of learning and memory, although the disease may manifest itself in other ways indicating altered cognitive ability. Throughout the disease the progressive loss of neurons and synapses in the cerebral cortex leads to gross atrophy of the neural tissue. Although the cause of Alzheimer's is unknown, many believe that inflammation plays an important role and clinical studies have shown that inflammation considerably contributes to the pathogenesis of the disease (Akiyama, et. al. (2000) Neurobiol Aging. 21:383-421.

In amyotrophic lateral sclerosis, a link between inflammation and the disease has been suggested (Centonze, et. al. (2007) Trends Pharm Sci 28:180-7). In addition, TNF-alpha mRNA has been found to be expressed in spinal cords of a transgenic mouse model for amyotrophic lateral sclerosis. Interestingly, the transcript was detected as early as prior to onset motor difficulties until death caused by ALS (Elliot (2001) Brain Res Mol Brain Res 95:172-8).

### Neurotoxins

Neurotoxins are toxins that specifically act upon neurons, their synapses, or the nervous system in its entirety. They are substances which cause damage to the structures of the brain which in turn leads to chronic disease. Neurotoxins include, for example, adrenergic neurotoxins, cholinergic neurotoxins, dopaminergic neurotoxins, excitotoxins, and other neurotoxins. Examples of adrenergic neurotoxins include N-(2-chloroethyl)-N-ethyl-2-bromobenzylamine hydrochloride. Examples of cholinergic neurotoxins include acetylethylcholine mustard hydrochloride. Examples of dopaminergic neurotoxins include 6-hydroxydopamine HBr (6-OHDA), 1-methyl-4-(2-methylphenyl)-1,2,3,6-tetrahydro-pyridine hydrochloride, 1-methyl-4-phenyl-2,3-dihydropyridinium perchlorate, N-methyl-4-phenyl-1,2,5,6tetrahydropyridine HCl (MPTP), 1-methyl-4-phenylpyridinium iodide (MPP+), paraquat, and rotenone. Examples of excitotoxins include NMDA and kainic acid.

MPTP, MPP+, paraquat, rotenone and 6-OHDA have been been shown to induce PD like symptoms in animal models. (See, K. Ossowska, et al., (2006). “Degeneration of dopaminergic mesocortical neurons and activation of compensatory processes induced by a long-term paraquat administration in rats: Implications for Parkinson's disease”. Neuroscience 141 (4): 2155-2165; and Caboni P, et al., (2004). “Rotenone, deguelin, their metabolites, and the rat model of Parkinson's disease”. Chem Res Toxicol 17 (11): 1540-8; Simon et al., Exp Brain Res, 1974, 20: 375-384; Langston et al., Science, 1983, 219: 979-980; Tanner, Occup Med, 1992, 7: 503-513; Liou et al., Neurology, 1997, 48: 1583-1588).

### SUMMARY OF THE INVENTION

Particular aspects provide methods for protecting against or reducing neurotoxicity of exposure to a neurotoxic agent, comprising administering to a subject in need thereof a therapeutically effective amount of an electrokinetically altered aqueous fluid comprising an ionic aqueous solution of charge-stabilized oxygen-containing nanostructures substantially having an average diameter of less than about 100 nanometers and stably configured in the ionic aqueous fluid in an amount sufficient to provide for neuroprotection against the neurotoxic agent, wherein an method for protecting against or reducing neurotoxicity of exposure to a neurotoxic agent is afforded. In certain aspects, the methods comprise protecting against or reducing loss of motor coordination in the subject exposed to the neurotoxin. In particular aspects, protecting or reducing neurotoxin-mediated neuronal apoptosis is afforded, and/or activating or inducing at least one of PI-3 kinase and Akt phosphorylation in neurons (e.g., of a subject) is afforded.

In particular aspects, the charge-stabilized oxygen-containing nanostructures are stably configured in the ionic aqueous fluid in an amount sufficient to provide, upon contact of a living cell by the fluid, modulation of at least one of cellular membrane potential and cellular membrane conductivity.

In particular embodiments, administering the fluid comprises administering the fluid prior to exposure to the neurotoxic agent.

In certain aspects, the charge-stabilized oxygen-containing nanostructures are the major charge-stabilized gas-containing nanostructure species in the fluid. In particular aspects, the percentage of dissolved oxygen molecules present in the fluid as the charge-stabilized oxygen-containing nanostructures is a percentage selected from the group consisting of greater than: 0.01%, 0.1%, 1%, 5%; 10%; 15%; 20%; 25%; 30%; 35%; 40%; 45%; 50%; 55%; 60%; 65%; 70%; 75%; 80%; 85%; 90%; and 95%. In certain aspects, the total dissolved oxygen is substantially present in the charge-stabilized oxygen-containing nanostructures. In certain embodiments, the charge-stabilized oxygen-containing nanostructures substantially have an average diameter of less than a size selected from the group consisting of: 90 nm; 80 nm; 70 nm; 60 nm; 50 nm; 40 nm; 30 nm; 20 nm; 10 nm; and less than 5 nm.

In certain aspects, the ionic aqueous solution comprises a saline solution, and/or is superoxygenated. In certain aspects, the fluid comprises a form of solvated electrons.

In particular aspects, alteration of the electrokinetically altered aqueous fluid comprises exposure of the fluid to hydrodynamically-induced, localized electrokinetic effects. In certain embodiments, exposure to the localized electrokinetic effects comprises exposure to at least one of voltage pulses and current pulses. In certain embodiments, exposure of the fluid to hydrodynamically-induced, localized electrokinetic effects, comprises exposure of the fluid to electrokinetic effect-inducing structural features of a device used to generate the fluid.

In certain aspects, the electrokinetically altered aqueous fluid modulates localized or cellular levels of nitric oxide. In particular aspects, the electrokinetically altered aqueous fluid promotes a localized decrease at the site of administration of at least one cytokine selected from the group consisting of: IL-1beta, IL-8, TNF-alpha, and TNF-beta.

Particular aspects of the methods comprise combination therapy, wherein at least one additional therapeutic agent is administered to the patient. In certain embodiments, the at least one additional therapeutic agent is selected from the group consisting of: adrenergic neurotoxins, cholinergic neurotoxins, dopaminergic neurotoxins, excitotoxins and chemotherapeutic agents.

In particular aspects, modulation of at least one of cellular membrane potential and cellular membrane conductivity comprises modulating at least one of cellular membrane structure or function comprising modulation of at least one of a conformation, ligand binding activity, or a catalytic activity of a membrane associated protein. In particular aspects, the membrane associated protein comprises at least one selected from the group consisting of receptors, transmembrane receptors, ion channel proteins, intracellular attachment proteins, cellular adhesion proteins, and integrins. In particular aspects, the transmembrane receptor comprises a G-Protein Coupled Receptor (GPCR). In particular aspects, the G-Protein Coupled Receptor (GPCR) interacts with a G protein a subunit. In particular aspects, the G protein a subunit comprises at least one selected from the group consisting of Gαₛ , Gαᵢ, Gα_{q}, and Gα₁₂. In particular aspects, the at least one G protein a subunit is Gα_{q}.

In certain aspects, modulating cellular membrane conductivity, comprises modulating whole-cell conductance. In particular embodiments, modulating whole-cell conductance, comprises modulating at least one voltage-dependent contribution of the whole-cell conductance.

In particular aspects, modulation of at least one of cellular membrane potential and cellular membrane conductivity comprises modulating intracellular signal transduction comprising modulation of a calcium dependant cellular messaging pathway or system. In particular aspects, modulation of at least one of cellular membrane potential and cellular membrane conductivity comprises modulating intracellular signal transduction comprising modulation of phospholipase C activity. In particular aspects, modulation of at least one of cellular membrane potential and cellular membrane conductivity comprises modulating intracellular signal transduction comprising modulation of adenylate cyclase (AC) activity. In particular aspects, modulation of at least one of cellular membrane potential and cellular membrane conductivity comprises modulating intracellular signal transduction associated with at least one condition or symptom selected from the group consisting of: chronic inflammation in the central nervous and brain, and acute inflammation in the central nervous and brain.

Certain aspects of the methods comprise administration to a cell network or layer, and further comprising modulation of an intercellular junction therein. In particular aspects, the intracellular junction comprises at least one selected from the group consisting of tight junctions, gap junctions, zona adherins and desmasomes. In certain embodiments, the cell network or layers comprises at least one selected from the group consisting of endothelial cell and endothelial-astrocyte tight junctions in CNS vessels, blood-cerebrospinal fluid tight junctions or barrier, pulmonary epithelium-type junctions, bronchial epithelium-type junctions, and intestinal epithelium-type junctions.

In particular aspects, the electrokinetically altered aqueous fluid is oxygenated, and the oxygen in the fluid is present in an amount of at least 8 ppm, at least 15, ppm, at least 25 ppm, at least 30 ppm, at least 40 ppm, at least 50 ppm, or at least 60 ppm oxygen at atmospheric pressure. In certain aspects, the amount of oxygen present in charge-stabilized oxygen-containing nanostructures of the electrokinetically-altered fluid is at least 8 ppm, at least 15, ppm, at least 20 ppm, at least 25 ppm, at least 30 ppm, at least 40 ppm, at least 50 ppm, or at least 60 ppm oxygen at atmospheric pressure.

In certain aspects, the electrokinetically altered aqueous fluid comprises at least one of a form of solvated electrons, and electrokinetically modified or charged oxygen species. In particular embodiments, the form of solvated electrons or electrokinetically modified or charged oxygen species are present in an amount of at least 0.01 ppm, at least 0.1 ppm, at least 0.5 ppm, at least 1 ppm, at least 3 ppm, at least 5 ppm, at least 7 ppm, at least 10 ppm, at least 15 ppm, or at least 20 ppm. In certain aspects, the electrokinetically altered oxygenated aqueous fluid comprises solvated electrons stabilized, at least in part, by molecular oxygen.

In particular aspects, the ability to modulate of at least one of cellular membrane potential and cellular membrane conductivity persists for at least two, at least three, at least four, at least five, at least 6, at least 12 months, or longer periods, in a closed gas-tight container.

In certain aspects, the membrane associated protein comprises CCR3. In particular aspecxts treating or administrating comprises administration by at least one of topical, inhalation, intranasal, oral and intravenous.

In certain embodiments, the charge-stabilized oxygen-containing nanostructures of the electrokinetically-alterd fluid comprise at least one salt or ion from Tables 1 and 2 disclosed herein.

Additional aspects provide a pharmaceutical composition, comprising an amount of an electrokinetically altered aqueous fluid comprising an ionic aqueous solution of charge-stabilized oxygen-containing nanostructures substantially having an average diameter of less than about 100 nanometers and stably configured in the ionic aqueous fluid in an amount sufficient for protecting against or reducing neurotoxicity of exposure to a neurotoxic agent.

Yet further aspects provide methods for preserving or improving motor coordination in a subject, having a neurodegenerative condition or disease, comprising administering to a subject having a neurodegenerative condition or disease characterized by loss of motor coordination, a therapeutically effective amount of an electrokinetically altered aqueous fluid comprising an ionic aqueous solution of charge-stabilized oxygen-containing nanostructures substantially having an average diameter of less than about 100 nanometers and stably configured in the ionic aqueous fluid in an amount sufficient to provide for preserving or improving motor coordination in the subject, wherein a method for preserving or improving motor coordination in a subject having a neurodegenerative condition or disease is afforded. In certain aspects, activation or induction of at least one of P1-3 kinase and Akt phosphorylation is afforded.

In particular aspects, the neurodegenerative condition or disease comprises at least one inflammatory neurrodegenerative condition or disease selected from the group consisting of multiple sclerosis, amyotrophic lateral sclerosis, Alzheimer's disease, Parkinson's disease, stroke/cerebral ischemia, head trauma, spinal cord injury, Huntington's disease, migraine, cerebral amyloid angiopathy, inflammatory neurodegenerative condition associated with AIDS, age-related cognitive decline; mild cognitive impairment and prion diseases in a mammal. Preferably, the inflammatory neurodegenerative condition or disease comprises at least one of multiple sclerosis, amyotrophic lateral sclerosis, Alzheimer's disease, Parkinson's disease.

Certain aspects of the methods comprise a synergistic or non-synergistic inhibition or reduction in inflammation by simultaneously or adjunctively treating the subject with another anti-inflammatory agent, for example, wherein said other anti-inflammatory agent comprises a steroid or glucocorticoid steroid. In certain aspects, the glucocorticoid steroid comprises Budesonide or an active derivative thereof.

Certain aspects of the methods comprise combination therapy, wherein at least one additional therapeutic agent is administered to the patient. In particular embodiments, the at least one additional therapeutic agent is selected from the group consisting of: glatiramer acetate, interferon-β, mitoxantrone, natalizumab, inhibitors of MMPs including inhibitor of MMP-9 and MMP-2, short-acting β₂-agonists, long-acting β₂-agonists, anticholinergics, corticosteroids, systemic corticosteroids, mast cell stabilizers, leukotriene modifiers, methylxanthines, β₂-agonists, albuterol, levalbuterol, pirbuterol, artformoterol, formoterol, salmeterol, anticholinergics including ipratropium and tiotropium; corticosteroids including beclomethasone, budesonide, flunisolide, fluticasone, mometasone, triamcinolone, methyprednisolone, prednisolone, prednisone; leukotriene modifiers including montelukast, zafirlukast, and zileuton; mast cell stabilizers including cromolyn and nedocromil; methylxanthines including theophylline; combination drugs including ipratropium and albuterol, fluticasone and salmeterol, budesonide and formoterol; antihistamines including hydroxyzine, diphenhydramine, loratadine, cetirizine, and hydrocortisone; immune system modulating drugs including tacrolimus and pimecrolimus; cyclosporine; azathioprine; mycophenolatemofetil; and combinations thereof.

In certain aspects, the at least one additional therapeutic agent is a TSLP and/or TSLPR antagonist. In particular embodiments, the TSLP and/or TSLPR antagonist is selected from the group consisting of neutralizing antibodies specific for TSLP and the TSLP receptor, soluble TSLP receptor molecules, and TSLP receptor fusion proteins, including TSLPR-immunoglobulin Fc molecules or polypeptides that encode components of more than one receptor chain.

In particular aspects, the charge-stabilized oxygen-containing nanostructures of the electrokinetically-alterd fluid comprise at least one salt or ion from Tables 1 and 2 disclosed herein.

### BRIEF DESCRIPTION OF THE DRAWINGS

FIGS. 1A-C demonstrate the results of a series of patch clamping experiments that assessed the effects of the electrokinetically generated fluid (e.g., RNS-60 and Solas) on epithelial cell membrane polarity and ion channel activity at two time-points (15 min (left panels) and 2 hours (right panels)) and at different voltage protocols.

FIGS. 2A-C show, in relation to the experiments relating to FIGS. 1A-C, the graphs resulting from the subtraction of the Solas current data from the RNS-60 current data at three voltage protocols (A. stepping from zero mV; B. stepping from −60 mV; C. stepping from −120 mV) and the two time-points (15 mins (open circles) and 2 hours (closed circles)).

FIGS. 3A-D demonstrate the results of a series of patch clamping experiments that assessed the effects of the electrokinetically generated fluid (e.g., Solas (panels A. and B.) and RNS-60 (panels C. and D.)) on epithelial cell membrane polarity and ion channel activity using different external salt solutions and at different voltage protocols (panels A. and C. show stepping from zero mV; panels B. and D. show stepping from −120 mV).

FIGS. 4A-D show, in relation to the experiments relating to FIGS. 3A-D, the graphs resulting from the subtraction of the CsCl current data (shown in FIG. 3) from the 20 mM CsCl₂ (diamonds) and 40 mM CsCl₂ (filled squares) current data at two voltage protocols (panels A. and C. stepping from zero mV; B. and D. stepping from −120 mV) for Solas (panels A. and B.) and Revera 60 (panels C. and D.).

FIG. 5 shows that the inventive electrokinetic fluid (RNS-60) was substantially efficacious in an art-recognized Experimental Autoimmune Encephalomyelitis (EAE) rat model of Multiple Sclerosis (MS).

FIG. 6 shows a schematic depiction of the EAE induction and treatment regimens used in the experiment shown in FIG. 7.

FIG. 7A is a graphical representation of the body weight (in grams) of the animals subjected to the EAE treatment regimen used in the experiment shown in FIGS. 5 and 6. FIG. 7B shows the calculated change in body weight (in percentage) of the animals subjected to the EAE treatment regimen.

FIGS. 8A-D show that the inventive electrokinetic fluid (RNS-60) had little affect on the level of total white blood cells (WBC), neutrophils, and lymphocytes when compared to the vehicle control during the EAE treatment regimen as used in the experiment shown in FIGS. 5 and 6. Panels A, B, C, and D show the results at study day 0, 7, 14, and 21, respectively.

FIGS. 9A-H (A-D) show the effect that the inventive electrokinetic fluid (RNS-60) had on cytokine levels 7 days (A-D) and 18 days (E-H) after the EAE treatment regimen as used in experiment shown in FIGS. 5 and 6 was initiated. Panels A and E show the levels of IL-17 after treatment. Panels B and F show the levels of IL-la after treatment. Panels C and G show the levels of IL-1β after treatment. Panels D and H show the levels of IL-4 after treatment.

FIG. 10 shows that the inventive electrokinetic fluid (RNS-60), but not control normal saline (NS), attenuates MPP+-induced expression of inducible nitric oxide synthase (iNOS) and interleukin-1β (IL-1β) in activated mouse microglial cells (BV-2 microglial cells).

FIGS. 11A and B show that RNS60, but not normal saline control (NS), suppresses fibrillar Aβ(1-42)-mediated apoptosis of human SHSY5Y neuronal cells (FIG. 11A) and primary human neurons (FIG. 11B). After differentiation, SHSY5Y cells were incubated with different concentrations of either RNS60 or NS for 1 h followed by insult with 1 μM fibrillar Aβ(1-42) peptides. After 18 h of treatment, apoptosis was monitored by TUNEL (Calbiochem). Aβ(42-1) peptides were also incubated as control. Results in each figure represent three independent experiments. DAPI staining was used to visualize the nucleus of cells.

FIG. 12 shows that RNS60, but not Vehicle control (Vehicle), is substantially efficacious in suppressing clinical score in a dose-responsive manner in an art-recognized experimental allergic encephalomyelitis (EAE) mouse MOG model of Multiple Sclerosis(MS).

Both high and low dose therapeutic daily administration of RNS-60, as well as the high dose administration of RNS-60 every three days (administration or RNS-60 in all instances beginning concomitant with first clinical signs), showed a marked decrease of clinical score (open diamonds=Vehicle control; open squares=dexamethasone positive control; light “x”s=low dose (0.09 ml RNS60) daily administration from onset of clinical signs; dark “x”s=high dose (0.2 ml RNS60) administration every three days from onset of clinical signs; and open triangles=high dose (0.2 ml RNS60) daily administration from onset of clinical signs).

FIGS. 13A-C show the results from two gel shift experiments (panels A and B) and a luciferase activity (reporter gene) assay (panel C) that examined the effects of RNS60 on the activation of NFκB in MBP-primed T cells.

FIGS. 14A-C are graphical representations scoring the coordinated movements of mice in a mouse model of PD, wherein the coordinated movements of mice improve when pre-treated with RNS60. Panels A and B show the total movement time and distance, respectively. Panel C shows the ability of the mice to keep their balance on a rotating rod.

FIGS. 15A and B are graphical representations scoring the striatum-dependent behaviors of mice in a mouse model of PD, wherein RNS60 treatment prevents the loss of striatum-dependent behaviors, stereotypy (grooming, Panel A) and rearing (vertical movements, Panel B).

FIGS. 16A-C show immunostaining with an anti-tyrosine hydroxylase antibody, tyrosine hydroxylase is the rate-limiting enzyme involved in dopamine synthesis, in the substantia nigra pars compacta. Panel A shows the normal staining of the anti-tyrosine hydroxylase antibody in the substantia nigra pars compacta. Panel B shows the effect that MPTP has on substantia nigra pars compacta, wherein staining of the substantia nigra pars compacta is reduced to approximately one-third. Panel C shows that RNS60 treatment rescues dopaminergic neurons in mice intoxicated with MPTP.

FIGS. 17A and B show the immunofluorescence analysis of phosphor-Akt in human neurons. The left, middle and right panels of FIG. 17A show the results from an experiment examining the effects of control, RNS60 (RIS60; 10%) and isotonic saline (10%), respectively, on Akt phosphorylation in primary neurons. Akt phosphorylation was monitored by double-label immunofluorescence using antibodies against β-tubulin and phospho-Akt. Beta-tubulin was used as a marker for neurons and DAPI staining was used to visualize the nucleus of cells. FIG. 17B shows that RNS60 suppresses fibrillar Aβ(1-42)-mediated apoptosis of human primary neurons and that this RNS60-mediated suppression can be blocked by the specific Akt inhibitor, AktI. Neurons preincubated with different concentrations of AktI for 30 min were treated with RNS60. After 1 h of incubation, cells were challenged with fibrillar Aβ1-42. After 12 h, neuronal apoptosis was monitored by TUNEL. Results represent three independent experiments. DAPI staining was used to visualize the nucleus of cells.

FIG. 18 is a graphical representation of the ratio between the amount of phosphorylated Akt to the total amount of Akt present in astrocytes when treated with either RNS60 or normal saline.

FIGS. 19A-B show the results from an experiment examining the effects of RNS60 on fibrillar Aβ(1-42)-mediated tau phosphorylation in primary neurons. Tau phosphorylation was monitored by double-label immunofluorescence using antibodies against β-tubulin and phospho-tau. Beta-tubulin was used as a marker for neurons and DAPI staining was used to visualize the nucleus of cells.

FIG. 20 shows that RNS60 suppresses fibrillar Aβ(1-42)-mediated apoptosis of human primary neurons and that this RNS60-mediated suppression can be blocked by an PI-3 kinase inhibitor (LY). Neurons preincubated with different concentrations of the PI-3 kinase inhibitor (LY) for 30 min were treated with RNS60. After 1 h of incubation, cells were challenged with fibrillar Aβ1-42. After 12 h, neuronal apoptosis was monitored by TUNEL.

FIG. 21 is, according to particular aspects, a schematic diagram of a signally pathway for the RNS60-mediated suppressive effect of fibrillar Aβ1-42-mediated apoptosis in neurons. Without being bound by mechanism, the schematic pathway shows an RNS60-mediated activation of PI-3 kinase, which in turns activates Akt via phosphorylation. According to further aspects, phosphorylated Akt mediates suppression of apoptosis.

### DETAILED DESCRIPTION OF THE INVENTION

Certain embodiments disclosed herein relate to providing compositions and methods of treatment of at least one symptom of an inflammatory neurodegenerative disease and/or multiple sclerosis by contacting the site or administering to a subject, a therapeutic composition comprising a novel electrokinetically-generated fluid. In certain specific embodiments, the electrokinetically-generated fluids comprise gas-enriched electrokinetically-generated fluid comprising oxygen-enriched water.

### Neuroprotective Compositions and Methods

Certain embodiments herein relate to therapeutic compositions and methods of treatment for a subject by preventing or alleviating at least one symptom associated with exposure to a neurotoxin or neurotoxic agent.

### Parkinson's Disease and Conditions

Certain embodiments herein relate to therapeutic compositions and methods of treatment for a subject by preventing or alleviating at least one symptom of Parkinson's Disease and/or an associated condition or disease.

In further embodiments herein relate to the therapeutic compositions and methods of treatment for preventing or alleviating complications related to Parkinson's Disease and/or an associated condition, including alleviating the symptoms of motor symptoms (e.g. tremor, rigidity, bradykinesia (slowness of movement) and gait impairment) and non-motor symptoms (e.g., such as cognitive deficits, depression, and sleep disorders).

### Electrokinetically-Generated Fluids:

“Electrokinetically generated fluid,” as used herein, refers to Applicants’ inventive electrokinetically-generated fluids generated, for purposes of the working Examples herein, by the exemplary Mixing Device described in detail herein (see also US200802190088 and WO2008/052143, both incorporated herein by reference in their entirety). The electrokinetic fluids, as demonstrated by the data disclosed and presented herein, represent novel and fundamentally distinct fluids relative to prior art non-electrokinetic fluids, including relative to prior art oxygenated non-electrokinetic fluids (e.g., pressure pot oxygenated fluids and the like). As disclosed in various aspects herein, the electrokinetically-generated fluids have unique and novel physical and biological properties including, but not limited to the following:

In particular aspects, the electrokinetically altered aqueous fluid comprise an ionic aqueous solution of charge-stabilized oxygen-containing nanostructures substantially having an average diameter of less than about 100 nanometers and stably configured in the ionic aqueous fluid in an amount sufficient to provide, upon contact of a living cell by the fluid, modulation of at least one of cellular membrane potential and cellular membrane conductivity.

In particular aspects, electrokinetically-generated fluids refers to fluids generated in the presence of hydrodynamically-induced, localized (e.g., non-uniform with respect to the overall fluid volume) electrokinetic effects (e.g., voltage/current pulses), such as device feature-localized effects as described herein. In particular aspects said hydrodynamically-induced, localized electrokinetic effects are in combination with surface-related double layer and/or streaming current effects as disclosed and discussed herein.

In particular aspects the administered inventive electrokinetically-altered fluids comprise charge-stabilized oxygen-containing nanostructures in an amount sufficient to provide modulation of at least one of cellular membrane potential and cellular membrane conductivity. In certain embodiments, the electrokinetically-altered fluids are superoxygenated (e.g., RNS-20, RNS-40 and RNS-60, comprising 20 ppm, 40 ppm and 60 ppm dissolved oxygen, respectively, in standard saline). In particular embodiments, the electrokinetically-altered fluids are not-superoxygenated (e.g., RNS-10 or Solas, comprising 10 ppm (e.g., approx. ambient levels of dissolved oxygen in standard saline). In certain aspects, the salinity, sterility, pH, etc., of the inventive electrokinetically-altered fluids is established at the time of electrokinetic production of the fluid, and the sterile fluids are administered by an appropriate route. Alternatively, at least one of the salinity, sterility, pH, etc., of the fluids is appropriately adjusted (e.g., using sterile saline or appropriate diluents) to be physiologically compatible with the route of administration prior to administration of the fluid. Preferably, and diluents and/or saline solutions and/or buffer compositions used to adjust at least one of the salinity, sterility, pH, etc., of the fluids are also electrokinetic fluids, or are otherwise compatible.

In particular aspects, the inventive electrokinetically-altered fluids comprise saline (e.g., one or more dissolved salt(s); e.g., alkali metal based salts (Li+, Na+, K+, Rb+, Cs+, etc.), alkaline earth based salts (e.g., Mg++, Ca++), etc., or transition metal-based positive ions (e.g., Cr, Fe, Co, Ni, Cu, Zn, etc.,), in each case along with any suitable anion components, including, but not limited to F—, Cl—, Br—, I—, PO4-, SO4-, and nitrogen-based anions. Particular aspects comprise mixed salt based electrokinetic fluids (e.g., Na+, K+, Ca++, Mg++, transition metal ion(s), etc.) in various combinations and concentrations, and optionally with mixtures of couterions. In particular aspects, the inventive electrokinetically-altered fluids comprise standard saline (e.g., approx. 0.9% NaCl, or about 0.15 M NaCl). In particular aspects, the inventive electrokinetically-altered fluids comprise saline at a concentration of at least 0.0002 M, at least 0.0003 M, at least 0.001 M, at least 0.005 M, at least 0.01 M, at least 0.015 M, at least 0.1 M, at least 0.15 M, or at least 0.2 M. In particular aspects, the conductivity of the inventive electrokinetically-altered fluids is at least 10 μS/cm, at least 40 μS/cm, at least 80 μS/cm, at least 100 μS/cm, at least 150 μS/cm, at least 200 μS/cm, at least 300 μS/cm, or at least 500 μS/cm, at least 1 mS/cm, at least 5, mS/cm, 10 mS/cm, at least 40 mS/cm, at least 80 mS/cm, at least 100 mS/cm, at least 150 mS/cm, at least 200 mS/cm, at least 300 mS/cm, or at least 500 mS/cm. In particular aspects, any salt may be used in preparing the inventive electrokinetically-altered fluids, provided that they allow for formation of biologically active salt-stabilized nanostructures (e.g., salt-stabilized oxygen-containing nanostructures) as disclosed herein.

According to particular aspects, the biological effects of the inventive fluid compositions comprising charge-stabilized gas-containing nanostructures can be modulated (e.g., increased, decreased, tuned, etc.) by altering the ionic components of the fluids, and/or by altering the gas component of the fluid.

According to particular aspects, the biological effects of the inventive fluid compositions comprising charge-stabilized gas-containing nanostructures can be modulated (e.g., increased, decreased, tuned, etc.) by altering the gas component of the fluid. In preferred aspects, oxygen is used in preparing the inventive electrokinetic fluids. In additional aspects mixtures of oxygen along with at least one other gas selected from Nitrogen, Oxygen, Argon, Carbon dioxide, Neon, Helium, krypton, hydrogen and Xenon. As described above, the ions may also be varied, including along with varying the gas constitutent(s).

Given the teachings and assay systems disclosed herein (e.g., cell-based cytokine assays, patch-clamp assays, etc.) one of skill in the art will readily be able to select appropriate salts and concentrations thereof to achieve the biological activities disclosed herein.

TABLE 1
Exemplary cations and anions.

| | | |
|---|---|---|
| Name | Formula | Other name(s) |

Common Cations:

| | | |
|---|---|---|
| Aluminum | Al⁺³ | |
| Ammonium | NH₄⁺ | |
| Barium | Ba⁺² | |
| Calcium | Ca⁺² | |
| Chromium(II) | Cr⁺² | Chromous |
| Chromium(III) | Cr⁺³ | Chromic |
| Copper(I) | Cu⁺ | Cuprous |
| Copper(II) | Cu⁺² | Cupric |
| Iron(II) | Fe⁺² | Ferrous |
| Iron(III) | Fe⁺³ | Ferric |
| Hydrogen | H⁺ | |
| Hydronium | H₃O⁺ | |
| Lead(II) | Pb⁺² | |
| Lithium | Li⁺ | |
| Magnesium | Mg⁺² | |
| Manganese(II) | Mn⁺² | Manganous |
| Manganese(III) | Mn⁺³ | Manganic |
| Mercury(I) | Hg₂⁺² | Mercurous |
| Mercury(II) | Hg⁺² | Mercuric |
| Nitronium | NO₂⁺ | |
| Potassium | K⁺ | |
| Silver | Ag⁺ | |
| Sodium | Na⁺ | |
| Strontium | Sr⁺² | |
| Tin(II) | Sn⁺² | Stannous |
| Tin(IV) | Sn⁺⁴ | Stannic |
| Zinc | Zn⁺² | |

Common Anions:

| | | | |
|---|---|---|---|
| Simple ions: | | | |
| Hydride | H⁻ | Oxide | O²⁻ |
| Fluoride | F⁻ | Sulfide | S²⁻ |
| Chloride | Cl⁻ | Nitride | N³⁻ |
| Bromide | Br⁻ | | |
| Iodide | I⁻ | | |
| Oxoanions: | | | |
| Arsenate | AsO₄³⁻ | Phosphate | PO₄³⁻ |
| Arsenite | AsO₃³⁻ | Hydrogen phosphate | HPO₄²⁻ |
| | | Dihydrogen phosphate | H₂PO₄⁻ |
| Sulfate | SO₄²⁻ | Nitrate | NO₃⁻ |
| Hydrogen sulfate | HSO₄⁻ | Nitrite | NO₂⁻ |
| Thiosulfate | S₂O₃²⁻ | | |
| Sulfite | SO₃²⁻ | | |
| Perchlorate | ClO₄⁻ | Iodate | IO₃⁻ |
| Chlorate | ClO₃⁻ | Bromate | BrO₃⁻ |
| Chlorite | ClO₂⁻ | | |
| Hypochlorite | OCl⁻ | Hypobromite | OBr⁻ |
| Carbonate | CO₃²⁻ | Chromate | CrO₄²⁻ |
| Hydrogen carbonate | HCO₃⁻ | Dichromate | Cr₂O₇²⁻ |
| or Bicarbonate | | | |
| Anions from Organic | | | |
| Acids: | | | |
| Acetate | CH₃COO⁻ | formate | HCOO⁻ |
| Others: | | | |
| Cyanide | CN⁻ | Amide | NH₂⁻ |
| Cyanate | OCN⁻ | Peroxide | O₂²⁻ |
| Thiocyanate | SCN⁻ | Oxalate | C₂O₄²⁻ |
| Hydroxide | OH⁻ | Permanganate | MnO₄⁻ |

TABLE 2
Exemplary cations and anions.

| | | |
|---|---|---|
| Formula | Charge | Name |

Monoatomic Cations

| | | |
|---|---|---|
| H⁺ | 1+ | hydrogen ion |
| Li⁺ | 1+ | lithium ion |
| Na⁺ | 1+ | sodium ion |
| K⁺ | 1+ | potassium ion |
| Cs⁺ | 1+ | cesium ion |
| Ag⁺ | 1+ | silver ion |
| Mg²⁺ | 2+ | magnesium ion |
| Ca²⁺ | 2+ | calcium ion |
| Sr²⁺ | 2+ | strontium ion |
| Ba²⁺ | 2+ | barium ion |
| Zn²⁺ | 2+ | zinc ion |
| Cd²⁺ | 2+ | cadmium ion |
| Al³⁺ | 3+ | aluminum ion |

Polyatomic Cations

| | | |
|---|---|---|
| NH₄⁺ | 1+ | ammonium ion |
| H₃O⁺ | 1+ | hydronium ion |

Multivalent Cations

| | | |
|---|---|---|
| Cr²⁺ | 2 | chromium(II) or chromous ion |
| Cr³⁺ | 3 | chromium(III)or chromic ion |
| Mn²⁺ | 2 | manganese(II) or manganous ion |
| Mn⁴⁺ | 4 | manganese(IV) ion |
| Fe²⁺ | 2 | iron(II) or ferrous ion |
| Fe³⁺ | 3 | iron(III) or ferric ion |
| Co²⁺ | 2 | cobalt(II) or cobaltous ion |
| Co³⁺ | 3 | cobalt(II) or cobaltic ion |
| Ni²⁺ | 2 | nickel(II) or nickelous ion |
| Ni³⁺ | 3 | nickel(III) or nickelic ion |
| Cu⁺ | 1 | copper(I) or cuprous ion |
| Cu²⁺ | 2 | copper(II) or cupric ion |
| Sn²⁺ | 2 | tin(II) or atannous ion |
| Sn⁴⁺ | 4 | tin(IV) or atannic ion |
| Pb²⁺ | 2 | lead(II) or plumbous ion |
| Pb⁴⁺ | 4 | lead(IV) or plumbic ion |

Monoatomic Anions

| | | |
|---|---|---|
| H⁻ | 1− | hydride ion |
| F⁻ | 1− | fluoride ion |
| Cl⁻ | 1− | chloride ion |
| Br⁻ | 1− | bromide ion |
| I⁻ | 1− | iodide ion |
| O²⁻ | 2− | oxide ion |
| S²⁻ | 2− | sulfide ion |
| N³⁻ | 3− | nitride ion |

Polyatomic Anions

| | | |
|---|---|---|
| OH⁻ | 1− | hydroxide ion |
| CN⁻ | 1− | cyanide ion |
| SCN⁻ | 1− | thiocyanate ion |
| C₂H₃O₂⁻ | 1− | acetate ion |
| ClO⁻ | 1− | hypochlorite ion |
| ClO₂⁻ | 1− | chlorite ion |
| ClO₃⁻ | 1− | chlorate ion |
| ClO₄⁻ | 1− | perchlorate ion |
| NO₂⁻ | 1− | nitrite ion |
| NO₃⁻ | 1− | nitrate ion |
| MnO₄²⁻ | 2− | permanganate ion |
| CO₃²⁻ | 2− | carbonate ion |
| C₂O₄²⁻ | 2− | oxalate ion |
| CrO₄²⁻ | 2− | chromate ion |
| Cr₂O₇²⁻ | 2− | dichromate ion |
| SO₃²⁻ | 2− | sulfite ion |
| SO₄²⁻ | 2− | sulfate ion |
| PO₃³⁻ | 3− | phosphite ion |
| PO₄³⁻ | 3− | phosphate ion |

The present disclosure sets forth novel gas-enriched fluids, including, but not limited to gas-enriched ionic aqueous solutions, aqueous saline solutions (e.g., standard aqueous saline solutions, and other saline solutions as discussed herein and as would be recognized in the art, including any physiological compatible saline solutions), cell culture media (e.g., minimal medium, and other culture media) useful in the treatment of diabetes or diabetes related disorders. A medium, or media, is termed “minimal” if it only contains the nutrients essential for growth. For prokaryotic host cells, a minimal media typically includes a source of carbon, nitrogen, phosphorus, magnesium, and trace amounts of iron and calcium. (Gunsalus and Stanter, The Bacteria, V. 1, Ch. 1 Acad. Press Inc., N.Y. (1960)). Most minimal media use glucose as a carbon source, ammonia as a nitrogen source, and orthophosphate (e.g., PO₄) as the phosphorus source. The media components can be varied or supplemented according to the specific prokaryotic or eukaryotic organism(s) grown, in order to encourage optimal growth without inhibiting target protein production. (Thompson et al., Biotech. and Bioeng. 27: 818-824 (1985)).

In particular aspects, the electrokinetically altered aqueous fluids are suitable to modulate ¹³C-NMR line-widths of reporter solutes (e.g., Trehelose) dissolved therein. NMR line-width effects are in indirect method of measuring, for example, solute ‘tumbling’ in a test fluid as described herein in particular working Examples.

In particular aspects, the electrokinetically altered aqueous fluids are characterized by at least one of: distinctive square wave voltametry peak differences at any one of −0.14V, −0.47V, −1.02V and −1.36V; polarographic peaks at −0.9 volts; and an absence of polarographic peaks at −0.19 and −0.3 volts, which are unique to the electrokinetically generated fluids as disclosed herein in particular working Examples.

In particular aspects, the electrokinetically altered aqueous fluids are suitable to alter cellular membrane conductivity (e.g., a voltage-dependent contribution of the whole-cell conductance as measure in patch clamp studies disclosed herein).

In particular aspects, the electrokinetically altered aqueous fluids are oxygenated, wherein the oxygen in the fluid is present in an amount of at least 15, ppm, at least 25 ppm, at least 30 ppm, at least 40 ppm, at least 50 ppm, or at least 60 ppm dissolved oxygen at atmospheric pressure. In particular aspects, the electrokinetically altered aqueous fluids have less than 15 ppm, less that 10 ppm of dissolved oxygen at atmospheric pressure, or approximately ambient oxygen levels.

In particular aspects, the electrokinetically altered aqueous fluids are oxygenated, wherein the oxygen in the fluid is present in an amount between approximately 8 ppm and approximately 15 ppm, and in this case is sometimes referred to herein as “Solas.”

In particular aspects, the electrokinetically altered aqueous fluid comprises at least one of solvated electrons (e.g., stabilized by molecular oxygen), and electrokinetically modified and/or charged oxygen species, and wherein in certain embodiments the solvated electrons and/or electrokinetically modified or charged oxygen species are present in an amount of at least 0.01 ppm, at least 0.1 ppm, at least 0.5 ppm, at least 1 ppm, at least 3 ppm, at least 5 ppm, at least 7 ppm, at least 10 ppm, at least 15 ppm, or at least 20 ppm.

In particular aspects, the electrokinetically altered aqueous fluids are suitable to alter cellular membrane structure or function (e.g., altering of a conformation, ligand binding activity, or a catalytic activity of a membrane associated protein) sufficient to provide for modulation of intracellular signal transduction, wherein in particular aspects, the membrane associated protein comprises at least one selected from the group consisting of receptors, transmembrane receptors (e.g., G-Protein Coupled Receptor (GPCR), TSLP receptor, beta 2 adrenergic receptor, bradykinin receptor, etc.), ion channel proteins, intracellular attachment proteins, cellular adhesion proteins, and integrins. In certain aspects, the effected G-Protein Coupled Receptor (GPCR) interacts with a G protein a subunit (e.g., Gαₛ , Gαᵢ, Gα_{q} , and Gα₁₂).

In particular aspects, the electrokinetically altered aqueous fluids are suitable to modulate intracellular signal transduction, comprising modulation of a calcium dependant cellular messaging pathway or system (e.g., modulation of phospholipase C activity, or modulation of adenylate cyclase (AC) activity).

In particular aspects, the electrokinetically altered aqueous fluids are characterized by various biological activities (e.g., regulation of cytokines, receptors, enzymes and other proteins and intracellular signaling pathways) described in the working Examples and elsewhere herein.

In particular aspects, the electrokinetically altered aqueous fluids display synergy with any one of erythropoietin, anti-apoptotics (TCH346, CEP-1347), antiglutamatergics, monoamine oxidase inhibitors (selegiline, rasagiline), promitochondrials (coenzyme Q10, creatine), calcium channel blockers (isradipine), alpha-synuclein, and growth factors (GDNF). In particular aspects, the electrokinetically altered aqueous fluids reduce DEP-induced TSLP receptor expression in bronchial epithelial cells (BEC) as shown in working Examples herein.

In particular aspects, the electrokinetically altered aqueous fluids inhibit the DEP-induced cell surface-bound MMP9 levels in bronchial epithelial cells (BEC) as shown in working Examples herein.

In particular aspects, the biological effects of the electrokinetically altered aqueous fluids are inhibited by diphtheria toxin, indicating that beta blockade, GPCR blockade and Ca channel blockade affects the activity of the electrokinetically altered aqueous fluids (e.g., on regulatory T cell function) as shown in working Examples herein.

In particular aspects, the physical and biological effects (e.g., the ability to alter cellular membrane structure or function sufficient to provide for modulation of intracellular signal transduction) of the electrokinetically altered aqueous fluids persists for at least two, at least three, at least four, at least five, at least 6 months, or longer periods, in a closed container (e.g., closed gas-tight container).

Therefore, further aspects provide said electrokinetically-generated solutions and methods of producing an electrokinetically altered oxygenated aqueous fluid or solution, comprising: providing a flow of a fluid material between two spaced surfaces in relative motion and defining a mixing volume therebetween, wherein the dwell time of a single pass of the flowing fluid material within and through the mixing volume is greater than 0.06 seconds or greater than 0.1 seconds; and introducing oxygen (O₂) into the flowing fluid material within the mixing volume under conditions suitable to dissolve at least 20 ppm, at least 25 ppm, at least 30, at least 40, at least 50, or at least 60 ppm oxygen into the material, and electrokinetically alter the fluid or solution. In certain aspects, the oxygen is infused into the material in less than 100 milliseconds, less than 200 milliseconds, less than 300 milliseconds, or less than 400 milliseconds. In particular embodiments, the ratio of surface area to the volume is at least 12, at least 20, at least 30, at least 40, or at least 50.

Yet further aspects, provide a method of producing an electrokinetically altered oxygenated aqueous fluid or solution, comprising: providing a flow of a fluid material between two spaced surfaces defining a mixing volume therebetween; and introducing oxygen into the flowing material within the mixing volume under conditions suitable to infuse at least 20 ppm, at least 25 ppm, at least 30, at least 40, at least 50, or at least 60 ppm oxygen into the material in less than 100 milliseconds, less than 200 milliseconds, less than 300 milliseconds, or less than 400 milliseconds. In certain aspects, the dwell time of the flowing material within the mixing volume is greater than 0.06 seconds or greater than 0.1 seconds. In particular embodiments, the ratio of surface area to the volume is at least 12, at least 20, at least 30, at least 40, or at least 50.

Additional embodiments provide a method of producing an electrokinetically altered oxygenated aqueous fluid or solution, comprising use of a mixing device for creating an output mixture by mixing a first material and a second material, the device comprising: a first chamber configured to receive the first material from a source of the first material; a stator; a rotor having an axis of rotation, the rotor being disposed inside the stator and configured to rotate about the axis of rotation therein, at least one of the rotor and stator having a plurality of through-holes; a mixing chamber defined between the rotor and the stator, the mixing chamber being in fluid communication with the first chamber and configured to receive the first material therefrom, and the second material being provided to the mixing chamber via the plurality of through-holes formed in the one of the rotor and stator; a second chamber in fluid communication with the mixing chamber and configured to receive the output material therefrom; and a first internal pump housed inside the first chamber, the first internal pump being configured to pump the first material from the first chamber into the mixing chamber. In certain aspects, the first internal pump is configured to impart a circumferential velocity into the first material before it enters the mixing chamber.

Further embodiments provide a method of producing an electrokinetically altered oxygenated aqueous fluid or solution, comprising use of a mixing device for creating an output mixture by mixing a first material and a second material, the device comprising: a stator; a rotor having an axis of rotation, the rotor being disposed inside the stator and configured to rotate about the axis of rotation therein; a mixing chamber defined between the rotor and the stator, the mixing chamber having an open first end through which the first material enters the mixing chamber and an open second end through which the output material exits the mixing chamber, the second material entering the mixing chamber through at least one of the rotor and the stator; a first chamber in communication with at least a majority portion of the open first end of the mixing chamber; and a second chamber in communication with the open second end of the mixing chamber.

Additional aspects provide an electrokinetically altered oxygenated aqueous fluid or solution made according to any of the above methods. In particular aspects the administered inventive electrokinetically-altered fluids comprise charge-stabilized oxygen-containing nanostructures in an amount sufficient to provide modulation of at least one of cellular membrane potential and cellular membrane conductivity. In certain embodiments, the electrokinetically-altered fluids are superoxygenated (e.g., RNS-20, RNS-40 and RNS-60, comprising 20 ppm, 40 ppm and 60 ppm dissolved oxygen, respectively, in standard saline). In particular embodiments, the electrokinetically-altered fluids are not-superoxygenated (e.g., RNS-10 or Solas, comprising 10 ppm (e.g., approx. ambient levels of dissolved oxygen in standard saline). In certain aspects, the salinity, sterility, pH, etc., of the inventive electrokinetically-altered fluids is established at the time of electrokinetic production of the fluid, and the sterile fluids are administered by an appropriate route. Alternatively, at least one of the salinity, sterility, pH, etc., of the fluids is appropriately adjusted (e.g., using sterile saline or appropriate diluents) to be physiologically compatible with the route of administration prior to administration of the fluid. Preferably, and diluents and/or saline solutions and/or buffer compositions used to adjust at least one of the salinity, sterility, pH, etc., of the fluids are also electrokinetic fluids, or are otherwise compatible therewith.

The present disclosure sets forth novel gas-enriched fluids, including, but not limited to gas-enriched ionic aqueous solutions, aqueous saline solutions (e.g., standard aqueous saline solutions, and other saline solutions as discussed herein and as would be recognized in the art, including any physiological compatible saline solutions), cell culture media (e.g., minimal medium, and other culture media).

### Neurotoxins:

By “toxic agent” or “neurotoxic agent” (neurotoxin) is meant a substance that through its chemical action injures, impairs, or inhibits the activity of a component of the nervous system. The list of neurotoxic agents that cause neuropathies is lengthy (see a list of exemplary neurotoxic agents provided in Table 3 below). Such neurotoxic agents include, but are not limited to, neoplastic agents such as vincristine, vinblastine, cisplatin, taxol, or dideoxy-compounds, e.g., dideoxyinosine; alcohol; metals; industrial toxins involved in occupational or environmental exposure; contaminants in food or medicinals; or over doses of vitamins or therapeutic drugs, e.g., antibiotics such as penicillin or chloramphenicol, or mega-doses of vitamins A, D, or B6.

Neurotoxicity may occur upon exposure to natural or artificial toxic substances (neurotoxins) that alters the normal activity of the nervous system in such a way as to cause damage to nervous tissue, and can eventually disrupt or kill neurons. Neurotoxicity can result from exposure to substances used in chemotherapy, radiation treatment, drug therapies, certain drug abuse, and organ transplants, as well as exposure to heavy metals, certain foods and food additives, pesticides, industrial and/or cleaning solvents, cosmetics, and some naturally occurring substances. Symptoms may appear immediately after exposure or be delayed. They may include limb weakness or numbness, loss of memory, vision, and/or intellect, uncontrollable obsessive and/or compulsive behaviors, delusions, headache, cognitive and behavioral problems and sexual dysfunction. Individuals with certain disorders may be especially vulnerable to neurotoxins.

According to particular embodiments, the compositions disclosed herein are used to prevent or ameliorate neurotoxicity caused by exposure to a variety of agents as discussed herein.

Certain toxins can cause peripheral neuropathy. Lead toxicity is associated with a motor neuropathy. Arsenic and mercury cause a sensory neuropathy. Thallium can cause a sensory and autonomic neuropathy. Several organic solvents and insecticides can also cause polyneuropathy. Alcohol is directly toxic to nerves and alcohol abuse is a major cause of neuropathy. The subject method can be used, in certain embodiments, as part of a broader detoxification program.

In still another embodiment, the methods and compositions of the present invention can be used for the treatment of neuropathies caused by drugs. Several drugs are known to cause neuropathy. They include, among others, vincristine and cisplatinum in cancer, nitrofurantoin, which is used in pyelonephritis, amiodarone in cardiac arrhythmias, disulfuram in alcoholism, ddC and ddI in AIDS, and dapsone which is used to treat leprosy. As above, the subject method can be used, in certain embodiments, as part of a broader detoxification program.

Another aspect of the invention provides a conjoint therapy wherein one or more other therapeutic agents are administered with the subject compound. Such conjoint treatment may be achieved by way of the simultaneous, sequential or separate dosing of the individual components of the treatment. Conjoint administration thus includes administration as part of the same pharmaceutical preparation, simultaneous administration of separate pharmaceutical preparations, as well as administration of separate pharmaceutical preparations at different times on the same day, adjacent days, or otherwise as part of a single therapeutic regimen. For example, the subject method can be carried out conjointly with other neuroprotective agents. The dosages recited herein would be adjusted to compensate for such additional components in the therapeutic composition. Progress of the treated patient can be monitored by conventional methods. In yet other embodiments, the subject method can be carried out conjointly with the administration of growth and/or trophic factors. For instance, the combinatorial therapy can include a trophic factor such as glial cell line-derived neurotrophic factor, nerve growth factor, cilliary neurotrophic factor, schwanoma-derived growth factor, glial growth factor, striatal-derived neuronotrophic factor, platelet-derived growth factor, brain-derived neurotrophic factor (BDNF), and scatter factor (HGF-SF). Antimitogenic agents can also be used, as for example, cytosine, arabinoside, 5-fluorouracil, hydroxyurea, and methotrexate.

Determination of a therapeutically effective amount and/or a prophylactically effective amount of administered composition of the invention, e.g., to be adequately neuroprotective, can be readily made one skilled in the art by the use of known techniques. The dosages may be varied depending upon the requirements of the patient in the judgment of the attending clinician, the severity of the condition being treated, the risk of further degeneration to the CNS, and the particular neurotoxin. In determining the therapeutically effective trophic amount or dose, and/or the prophylactically effective amount or dose, a number of factors are considered by the attending clinician, including, but not limited to: the specific cause of the degenerative state and its likelihood of recurring or worsening; pharmacodynamic characteristics of the particular neurotoxic agent; the desired time course of treatment; the species of mammal; its size, age, and general health; the response of the individual patient; the particular compound administered; the bioavailability characteristics of the preparation administered; the dose regimen selected; the kind of concurrent treatment; and other relevant circumstances.

Treatment can be initiated with smaller dosages that are less than the optimum dose. Thereafter, the dosage may be increased by small increments until the optimum effect under the circumstances is reached. For convenience, the total daily dosage may be divided and administered in portions during the day if desired. A therapeutically effective trophic amount and a prophylactically effective neuroprotective amount of therapeutic composition, for instance, is expected to vary depending on the route of administration, and other factors as discussed above.

Compositions effective for the prevention or treatment of degeneration of neurons (e.g., dopaminergic neurons and motoneurons and the like) in animals, e.g., dogs, rodents, may also be useful in treatment of disorders in humans. Those skilled in the art of treating in such disorders in humans will be guided, from the data obtained in animal studies, to the correct dosage and route of administration of the compound to humans. In general, the determination of dosage and route of administration in humans is expected to be similar to that used to determine administration in animals.

The identification of those patients who are in need of prophylactic treatment for disorders marked by degeneration of neurons (e.g. dopaminergic neurons and/or motoneurons and the like) is well within the ability and knowledge of one skilled in the art. Certain of the methods for identification of patients that are at risk and that can be treated by the subject method are appreciated in the medical arts, such as family history of the development of a particular disease state and the presence of risk factors associated with the development of that disease state in the subject patient. Risk of environmental (e.g., chemical) exposure. A clinician skilled in the art can readily identify such candidate patients, by the use of, for example, clinical tests, physical examination, medical/family history, vocation/occupation, etc.

Protecting soldiers against any kind of threat and preserving their ability to fight has become a major concern of armies. Nerve gas (e.g., sarin, soman or Vx) is one such threat. One class of nerve agents (also known as nerve gases) are phosphorus-containing organic chemicals (organophosphates) that block acetylcholinesterase, an enzyme that normally relaxes the activity of acetylcholine, a neurotransmitter. There are two main classes of nerve agents, G agents (e.g., GA, tabun or ethyl N,N-dimethylphosphoramidocyanidate; GB, sarin or O-isopropyl methylphosphonofluoridate; GD, soman or O-pinacolyl methylphosphonofluoridate; GF, cyclosarin or cyclohexyl methylphosphonofluoridate; GV, P-[2-(dimethylamino)ethyl]-N,N-dimethylphosphonamidic fluoride)) and V agents (VE, S-(diethylamino)ethyl O-ethyl ethylphosphonothioate; VG, Amiton or Tetram or O,O-diethyl-S-[2-(diethylamino)ethyl]phosphorothioate; VM, phosphonothioic acid, methyl-, S-(2-(diethylamino)ethyl) O-ethyl ester); VX, O-ethyl-S-[2(diisopropylamino)ethyl]methylphosphonothiolate). A third group of agents, the Novichok agents, are organophosphate compounds that inhibit the enzyme cholinesterase, preventing the normal breakdown of acetylcholine.

Insecticides, the organophosphates, such as dichlorvos, malathion and parathion, are nerve agents.

TABLE 3
Neurotoxic Agents

| AGENT | ACTIVITY |
|---|---|
| actazolimide | Diuretic |
| Acrylamide | flocculant, grouting agent |
| adriamycin | Antineoplastic |
| alcohol (i.e. ethanol) | solvent, recreational drug |
| almitine | respiratory stimulant |
| amiodarone | Antiarrthymic |
| amphotericin | Antimicrobial |
| arsenic | herbicide, insecticide |
| aurothioglucose | Antirheumatic |
| barbiturates | anticonvulsive, sedative |
| buckthorn | toxic berry |
| carbimates | Insecticide |
| carbon disulfide | industrial applications |
| chlorarnphenicol | Antibacterial |
| chloroquine | Antimalarial |
| chlorestyramine | Antihyperlipoproteinemic |
| cisplatin | Antineoplastic |
| clioquinol | amebicide, antibacterial |
| colestipol | Antihyperlipoproteinemic |
| colchicine | gout suppressant |
| colistin | Antimicrobial |
| cycloserine | Antibacterial |
| cytarabine | Antineoplastic |
| dapsone | dermatological ie- |
| | leprosy |
| dideoxycytidine | Anatineoplastic |
| dideoxyinosine | Antineoplastic |
| dideoxythymidine | Antiviral |
| disulfiram | Antialcohol |
| doxorubicin | Antineoplastic |
| ethambutol | Antibacterial |
| ethionamide | Antibacterial |
| glutethimide | sedative, hypnotic |
| gold | Antirheumatic |
| hexacarbons | Solvents |
| hormonal contraceptives | |
| hexamethylolmelamine | fireproofing, crease |
| | proofing |
| hydralazine | Antihypertensive |
| hydroxychloroquine | Antirheumatic |
| imipramine | antidepressant |
| indolmethacin | anti-inflammatory |
| inorganic lead | toxic metal in |
| | paint, etc. |
| iso-niazid | antituberculousis |
| lithium | antidepressant |
| methylmercury | industrial waste |
| metformin | antidiabetic |
| methylhydrazine | synthetic |
| | intermediate |
| metronidazole | antiprotozoal |
| misonidazole | radiosensitizer |
| nitrofurantoin | urinary antiseptic |
| nitrogen mustard | antineoplastic, |
| | nerve gas |
| nitous oxide | anesthetic |
| organophosphates | insecticides |
| ospolot | anticonvulsant |
| penicillin | antibacterial |
| perhexiline | antiarrhythmic |
| perhexiline | antiarrythmic |
| maleate | |
| phenytoin | anticonvulsant |
| platnim | drug component |
| primidone | anticonvulsant |
| procarbazine | antineoplastic |
| pyridoxine | vitamin B6 |
| sodium cyanate | antisickling |
| streptomycin | antimicrobial |
| sulphonamides | antimicrobial |
| suramin | anteneoplastic |
| tamoxifen | antineoplastic |
| taxol | antineoplastic |
| thalidomide | antileprous |
| thallium | rat poison |
| triamterene | diuretic |
| trimethyltin | toxic metal |
| L-trypophan | health food |
| | additive |
| vincristine | Antineoplastic |
| vinblastine | Antineoplastic |
| vindesine | Antineoplastic |
| vitamine A or D | mega doses |

In particular embodiments, the methods and compositions of the present invention can be used for the prevention or amelioration of chemotheraphy induced neurotoxicity (see, e.g, U.S. Pat. No. 7,129,250, (published as 2004/0220202), which is incorporated by reference herein in its entirety, and in particular for its teachings of exemplary neurotoxins).

For example, in particular embodiments, the methods and compositions of the present invention can be used with an anti-cancer agent such as an anti-cancer drug, a cytokine, and/or supplementary potentiating agent(s). The use of cocktails in the treatment of cancer is routine. In this embodiment, a common administration vehicle (e.g., orally available or injectable solution, etc.) could contain both a compositions of the present invention and the anti-cancer drug and/or supplementary potentiating agent. Thus, cocktails comprising compositions of the present invention as well as other compounds are within the scope of the invention.

Compounds having anti-neoplastic properties include, but are not limited to: Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanin; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Imofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycinl, Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Other anti-neoplastic compounds include: 20epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emiteftir; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormiaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based inmmune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Anti-cancer supplementary potentiating agents include, but are not limited to: tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca++ antagonists (e.g., yerapamil, nifedipine, nitrendipine and caroverine); calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL

### Inflammation

Inflammation may occur as a defensive response to invasion of the subject by foreign material, particularly of microbial origin. Additionally, mechanical trauma, toxins, and neoplasia may induce inflammatory responses. The accumulation and subsequent activation of leukocytes are central events in the pathogenesis of most forms of inflammation. Inflammation deficiencies can compromise the host, leaving it susceptible to worsening infection or trauma. Excessive inflammation, such as prolonged inflammatory responses, may lead to inflammatory diseases including but not limited to diabetes, arteriosclerosis, cataracts, chronic skin disorders, reperfusion injury, and cancer, to post-infectious syndromes such as in infectious meningitis, rheumatic fever, and to rheumatic diseases such as systemic lupus erythematosus and rheumatoid arthritis. These diseases affect millions of people worldwide every year, and lead to increased mortality and morbidity. The commonality of the inflammatory response in these varied disease processes makes its regulation a major element in the prevention, or treatment of human disease.

Overproduction of pro-inflammatory cytokines has been implicated in the pathogenesis of numerous inflammatory and autoimmune diseases. Secretion of TNFα is a primary event in the initiation of the inflammatory cascade (Brennan F. M., et. al. Lancet, 1989, 2:244-7; Haworth C, et. al. Eur. J. Immunol. 1991, 21:2575-2579) and directly contributes to the initiation and maintenance of these diseases. Other cytokines also play a role, including interleukin 1β(IL-1β), IL-6, IL-8, IL-12 nitric oxide (NO), IFN-γ, granulocyte colony stimulating factor (G-CSF), granulocyte macrophage-colony stimulating factor (GM-CSF), and IL-10. Certain of these cytokines (e.g. IL-8) may increase or exacerbate an inflammatory response, while others (e.g. IL-10) may decrease or alleviate the inflammatory response.

Cells of the immune system, macrophages in particular, secrete many of these cytokines in response to activating stimuli. Target cells of the cytokines may be localized in any body compartment and may act via long-distance mechanisms, or may act on neighboring cells. Thus, cytokines may regulate inflammation in a localized or systemic manner.

### Metalloproteinases

Metalloproteinases are a superfamily of proteinases (enzymes) classified into families and subfamilies as described, for example, in N. M. Hooper FEBS Letters 354:1-6, 1994. Examples of metalloproteinases include the matrix metalloproteinases (MMPs) such as the collagenases (MMP1, MMP8, MMP13), the gelatinases (MMP2, MMP9), the stromelysins (MMP3, MMP10, MMP II), matrilysin (MMPI), metalloelastase (MMP12), enamelysin (MMP19), the MT-MMPs (MMP14, MMP15, MMP16, MMP17); the reprolysin or adamalysin or MDC family which includes the secretases and sheddases such as TNF converting enzymes (ADAM10 and TACE); the astacin family which include enzymes such as procollagen processing proteinase (PCP); and other metalloproteinases such as aggrecanase, the endothelin converting enzyme family and the angiotensin converting enzyme family. Collectively, the metalloproteinases are known to cleave a broad range of matrix substrates such as collagen, proteoglycan and fibronectin. Metalloproteinases are implicated in the processing, or secretion, of biological important cell mediators, such as tumour necrosis factor (TNF); and the post translational proteolysis processing, or shedding, of biologically important membrane proteins, such as the low affinity IgE receptor CD23 (see, e.g., N. M. Hooper et al., Biochem. J. 321:265-279, 1997).

Not surprisingly, therefore, metalloproteinases are believed to be important in many physiological disease processes that involve tissue remodeling (e.g., embryonic development, bone formation, uterine remodelling during menstruation, etc.). Moreover, inhibition of the activity of one or more metalloproteinases may well be of benefit in these diseases or conditions, for example: various inflammatory and allergic diseases such as, inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis); in tumour metastasis or invasion; in disease associated with uncontrolled degradation of the extracellular matrix such as osteoarthritis; in bone resorptive disease (such as osteoporosis and Paget's disease); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); Alzheimer's disease; extracellular matrix remodelling observed in cardiovascular diseases such as restenosis and atherosclerosis; asthma; rhinitis; and chronic obstructive pulmonary diseases (COPED).

MMP12, also known as macrophage elastase or metalloelastase, was initially cloned in the mouse (Shapiro et al., Journal of Biological Chemistry 267: 4664, 1992) and has also been cloned in man by the same group in 1995. MMP12 is preferentially expressed in activated macrophages, and has been shown to be secreted from alveolar macrophages from smokers (Shapiro et al, 1993, Journal of Biological Chemistry, 268: 23824) as well as in foam cells in atherosclerotic lesions (Matsumoto et al, Am. J. Pathol. 153: 109, 1998). A mouse model of COPD is based on challenge of mice with cigarette smoke for six months, two cigarettes a day six days a week. Wild-type mice developed pulmonary emphysema after this treatment. When MMP12 knock-out mice were tested in this model they developed no significant emphysema, strongly indicating that MMP12 is a key enzyme in the COPD pathogenesis. The role of MMPs such as MMP12 in COPD (emphysema and bronchitis) is discussed in Anderson and Shinagawa, 1999, Current Opinion in Anti-inflammatory and Immunomodulatory Investigational Drugs 1(1): 29-38. It was recently discovered that smoking increases macrophage infiltration and macrophage-derived MMP-12 expression in human carotid artery plaques (Matetzky S, Fishbein M C et al., Circulation 102:(18), 36-39 Suppl. S, Oct. 31, 2000).

MMP9-(Gelatinase B; 92 kDa-TypeIV Collagenase; 92 kDa Gelatinase) is a secreted protein which was first purified, then cloned and sequenced, in 1989 (S. M. Wilhelm et al., J. Biol. Chem. 264 (29): 17213-17221, 1989; published erratum in J. Biol. Chem. 265 (36): 22570, 1990) (for review of detailed information and references on this protease see T. H. Vu & Z. Werb (1998) (In: Matrix Metalloproteinases, 1998, edited by W. C. Parks & R. P. Mecham, pp. 115-148, Academic Press. ISBN 0-12-545090-7). The expression of MMP9 is restricted normally to a few cell types, including trophoblasts, osteoclasts, neutrophils and macrophages (Vu & Werb, supra). However, the expression can be induced in these same cells and in other cell types by several mediators, including exposure of the cells to growth factors or cytokines These are the same mediators often implicated in initiating an inflammatory response. As with other secreted MMPs, MMP9 is released as an inactive Pro-enzyme, which is subsequently cleaved to form the enzymatically active enzyme. The proteases required for this activation in vivo are not known. The balance of active MMP9 versus inactive enzyme is further regulated in vivo by interaction with TIMP-1 (Tissue Inhibitor of Metalloproteinases-1), a naturally-occurring protein. TIMP-1 binds to the C-terminal region of MMP9, leading to inhibition of the catalytic domain of MMP9. The balance of induced expression of ProMMP9, cleavage of Pro-to active MMP9 and the presence of TIMP-1 combine to determine the amount of catalytically active MMP9 which is present at a local site. Proteolytically active MMP9 attacks substrates which include gelatin, elastin, and native Type IV and Type V collagens; it has no activity against native Type I collagen, proteoglycans or laminins There has been a growing body of data implicating roles for MMP9 in various physiological and pathological processes. Physiological roles include the invasion of embryonic trophoblasts through the uterine epithelium in the early stages of embryonic implantation; some role in the growth and development of bones; and migration of inflammatory cells from the vasculature into tissues.

MMP9 release, measured using enzyme immunoassay, was significantly enhanced in fluids and in AM supernatants from untreated asthmatics compared with those from other populations (Am. J. Resp. Cell & Mol. Biol., 5:583-591, 1997). Also, increased MMP9 expression has been observed in certain other pathological conditions, thereby implicating MMP9 in disease processes such as COPD, arthritis, tumour metastasis, Alzheimer's disease, multiple sclerosis, and plaque rupture in atherosclerosis leading to acute coronary conditions such as myocardial infarction (see also WO07087637A3, incorporated herein by reference).

Recently, it has been demonstrated that the levels of MMP-9 are significantly increased in patients with stable asthma and even higher in patients with acute asthmatic patients compared with healthy control subjects. MMP-9 plays a crucial role in the infiltration of airway inflammatory cells and the induction of airway hyperresponsiveness indicating that MMP-9 may have an important role in inducing and maintaining asthma (Vignola et al., Sputum metalloproteinase-9/tissue inhibitor of metalloproteinase-1 ratio correlates with airflow obstruction in asthma and chronic bronchitis, Am J Respir Crit Care Med 158:1945-1950, 1998; Hoshino et al., Inhaled corticosteroids decrease subepithelial collagen deposition by modulation of the balance between matrix metalloproteinase-9 and tissue inhibitor of metalloproteinase-1 expression in asthma, J Allergy Clin Immunol 104:356-363, 1999; Simpson et al., Differential proteolytic enzyme activity in eosinophilic and neutrophilic asthma, Am J Respir Crit Care Med 172:559-565,2005; Lee et al., A murine model of toluene diisocyanate-induced asthma can be treated with matrix metalloproteinase inhibitor, J Allergy Clin Immunol 108:1021-1026, 2001; and Lee et al., Matrix metalloproteinase inhibitor regulates inflammatory cell migration by reducing ICAM-1 and VCAM-1 expression in a murine model of toluene diisocyanate-induced asthma, J Allergy Clin Immunol 2003;111:1278-1284).

### MMP Inhibitors:

A number of metalloproteinase inhibitors are known (see, for example, the reviews of MMP inhibitors by Beckett R. P. and Whittaker M., 1998, Exp. Opin. Ther. Patents, 8(3):259-282; and by Whittaker M. et al, 1999, Chemical Reviews 99(9):2735-2776). WO 02/074767 discloses hydantoin derivatives of formula that are useful as MMP inhibitors, particularly as potent MMP12 inhibitors. U.S. patent application Ser. No. 11/721,590 (published as 20080032997) discloses a further group of hydantoin derivatives that are inhibitors of metalloproteinases and are of particular interest in inhibiting MMPs such as MMP 12 and MMP9. Novel triazolone derivatives for inhibiting MMPs such as MMP12 and MMP9 are disclosed in U.S. patent application Ser. No. 10/593,543 (published as 20070219217). Additional MMP12 and MMP9 inhibitors are disclosed in Ser. No. 11/509,490 (published as 20060287338) (see also Ser. No. 10/831,265 (published as 20040259896)).

Additionally, two compounds, 4-(4-phenoxyphenylsulfonyl)butane-1,2-dithiol (1) and 5-(4-phenoxyphenylsulfonyl)pentane-1,2-dithiol (2), have been shown to bind selectively and inhibit potently MMP-2 and MMP-9 (Bernardo, et. al (2002) J. Biol. Chem. 277:11201-11207). These two compounds may have significant use in the clinic to inhibit MMP-2 and -9 and therefore lessen inflammation. In addition, the use of certain tetracycline antibiotics (e.g., Minocycline and Doxycycline) at sub-antibiotic levels has been shown to effectively inhibit MMP activity. Certain aspects of this invention include using the inventive fluids in combination with sub-antibiotic levels useful to inhibit MMP.

### Methods of Treatment

The term “treating” refers to, and includes, reversing, alleviating, inhibiting the progress of, or preventing a disease, disorder or condition, or one or more symptoms thereof; and “treatment” and “therapeutically” refer to the act of treating, as defined herein.

A “therapeutically effective amount” is any amount of any of the compounds utilized in the course of practicing the invention provided herein that is sufficient to reverse, alleviate, inhibit the progress of, or prevent a disease, disorder or condition, or one or more symptoms thereof.

Certain embodiments herein relate to therapeutic compositions and methods of treatment for a subject by preventing or alleviating at least one symptom associated with exposure to a neurotoxin. For example, the therapeutic compositions and/or methods disclosed herein may be useful for treating or preventing one or more condition or disease selected from the group consisting multiple sclerosis (MS), Parkinson's disease, amyloidosis (e g Alzheimer's disease), amyotrophic lateral sclerosis (ALS), prion diseases, and HIV-associated dementia.

Many conditions or diseases associated with inflammation have been treated with steroids, methotrexate, immunosuppressive drugs including cyclophosphamide, cyclosporine, azathioprine and leflunomide, nonsteroidal anti-inflammatory agents such as aspirin, acetaminophen and COX-2 inhibitors, gold agents and anti-malarial treatments. These drugs have a variety of disadvantages, and adverse reactions including injection site reactions, rash, upper respiratory infections, autoimmune disorders and increased susceptibility to infections. In addition, many anti-inflammatory pharmaceutical drugs require intravenous (IV) or subcutaneous (SC) administration, as opposed to more convenient and compliant oral or topical dermal routes. Accordingly, a need still exists for the development of novel medicaments and treatment methods for conditions and diseases relating to inflammation.

### Combination Therapy:

Additional aspects provide the herein disclosed inventive methods, further comprising combination therapy, wherein at least one additional therapeutic agent is administered to the patient. In certain aspects, the at least one additional therapeutic agent is selected from the group consisting of any one of erythropoietin, anti-apoptotics (TCH346, CEP-1347), antiglutamatergics, monoamine oxidase inhibitors (selegiline, rasagiline), promitochondrials (coenzyme Q10, creatine), calcium channel blockers (isradipine), alpha-synuclein, and/or growth factors (GDNF).

### Anti-Inflammatory Activity of the Electrokinetically-Generated Gas-Enriched Fluids and Solutions:

According to certain aspects of the present invention, the gas-enriched fluids and/or solutions disclosed herein have anti-inflammatory properties and effects, and can be used as anti-inflammatory agents for the treatment of subjects afflicted by diseases or disorders relating to inflammatory neurodegeneration. Previous results showed that the inventive oxygen-enriched fluid (water) affected a down regulation of particular cytokines, especially IL-6, IL-8, and IL-1β in cytokine profiles in stimulated lymphocytes from a healthy blood donor.

Increased production of pro-inflammatory cytokines has been implicated in the pathogenesis of numerous inflammatory and autoimmune diseases. Secretion of TNFα is a primary event in the initiation of the inflammatory cascade (Brennan F. M., et. al. Lancet, 1989, 2:244-7; Haworth C, et. al. Eur. J. Immunol. 1991, 21:2575-2579) and directly contributes to the initiation and maintenance of inflammatory and autoimmune diseases. Other pro-inflammatory cytokines also play a role, including interleukin 1β(IL-1β), IL-6, IL-8, IL-12 nitric oxide, IFN-γ and GM-CSF, while anti-inflammatory cytokines such as IL-10 may reduce disease. Cells of the immune system, macrophages in particular, secrete many of these cytokines in response to activating stimuli.

A variety of cell types are involved in the inflammatory process. Overproduction of TNFα by monocytes, macrophages and other immune cells is a key element in the pathogenesis of a multitude of diseases. Macrophages and T-cells in particular play a central role in the initiation and maintenance of the immune response. Once activated by pathological or immunogenic stimuli, macrophages respond by releasing a host of cytokines, including TNF-α, IL-1β, IL-8, IL-12, nitric oxide (NO), IL-6, GM-CSF, G-CSF, M-CSF and others. T-cells release IL-2, IL-4, INF-γ, and other inflammatory cytokines These cytokines activate other immune cells and some can also act as independent cytotoxic agents. Excessive release of macrophage and T-cell derived inflammatory mediators can particularly lead to damage of normal cells and surrounding tissues.

Pro-inflammatory cytokines have been implicated in HIV-AIDS, and other viral infections including the cytomegalovirus, influenza virus and the herpes family of viruses. TNFα enhances the basal activity of the major immediate early enhancer/promoter of human cytomegalovirus and may play a role in reactivation of latent HCMV infection in premonocytic cells (Prosch S., et. al. Virology 1995, 208:197-206).

Additionally, a number of inflammatory cytokines contribute to mortality in patients suffering from sepsis or endotoxic shock. For example, TNFαand IL-1β have a well-established central role in sepsis, septic shock and endotoxic shock. Increased levels of these cytokines are associated with fever, hypotension and shock (Smith J. W. et. al. J. Clin. Oncol. 1992, 10:1141-1152; Chapman P. B., et. al. J. Clin. Oncol. 1987, 5:1942-1951) together with the induction of gene expression for phospholipase A2 (Gronich J., et. al. J. Clin. Invest. 1994, 93:1224-1233) and NO synthase.

The induction of NO from smooth muscle cells mediates decreased mean arterial pressure and systemic vascular resistance during septic shock, suggesting a fundamental role for NO. Thus, therapies that target downregulatory effects on IL-8, IL-1β, and NO could be beneficial in the treatment of inflammatory diseases or disorders, including sepsis, septic shock, and endotoxic shock.

Overproduction of TNFα contributes to the clinical features of numerous autoimmune diseases such as diabetes and rheumatoid arthritis. Systemic lupus erythematosus (SLE) is also precipitated by increased IL-1β and TNFα levels. Within lupus patients, serum C-reactive protein, IL-1.beta and TNFα levels were higher than in controls, suggesting that an increased inflammatory response plays a role in the disease (Liou L. B. Clin. Exp. Rheumatol. 2001, 19:515-523). A study of patients with one form of SLE, neuropsychiatric lupus erythematosus (NPLE), showed that the number of peripheral blood mononuclear cells expressing mRNA for TNFα as well as the cerebrospinal fluid level of NO metabolites correlated with NPLE disease severity (Svenungsson E., et al. Ann. Rheum. Dis. 2001, 60:372-9).

IL-1 and TNFα play a central role in various acute as well as chronic responses in animal models. Additionally, IL-11, IFNα and IFNβ may also up-regulate inflammatory reactions. Conversely, several cytokines may be involved in down-regulation of inflammatory responses (i.e. IL-4, IL-10, IL-13, among others). As set forth in Example 1, cells contacted with the inventive gas-enriched fluid showed an increase in IFN-γ levels with T3 antigen than in the control culture media with T3 antigen, while IL-8 was lower in the inventive gas-enriched culture media with T3 antigen than in the control culture media with T3 antigen. Additionally, IL-6, IL-8, and TNF-α levels were lower in the inventive gas-enriched media with PHA, than in the control media with PHA, while IL-1β levels were lower in the inventive gas-enriched fluid with PHA when compared with control media with PHA. In the inventive gas-enriched media alone, IFN-γ levels were higher than in control media. These results are consistent with an anti-inflammatory microenvironment.

NO is recognized as a mediator and regulator of inflammatory responses. It possesses cytotoxic properties toward pathogens, but can also have deleterious effects on the subject's own tissues. (Korhonen et al., Curr Drug Targets Inflamm Allergy 4(4): 471-9, 2005). NO reacts with soluble guanylate cyclase to form cyclic guanosine monophosphate (cGMP), which mediates many of the effects of NO. NO can also interact with molecular oxygen and superoxide anion to produce reactive oxygen species that can modify various cellular functions. These indirect effects of NO have a significant role in inflammation, where NO is produce in high amounts by inducible NO synthase (iNOS) and reactive oxygen species are synthesized by activated inflammatory cells.

NO can be produced by keratinocytes, fibroblasts, endothelial cells, and possibly others. Some of the vascular actions of NO include vasodilation, inhibiting platelet adhesion to the vascular endothelium, inhibiting leukocyte adhesion to the vascular endothelium, and scavenging superoxides. (Shah et al., Env. Health Persp. v. 106 (5): 1139-1143.)

Furthermore, inhibition of NO synthesis has been shown to delay wound contraction, alter collagen organization, and alter neoepidermis thickness. (Amadeu and Costa, J. Cutan. Pathol. 33: 465-473, 2006.) Mast cell migration and angiogenesis in wounds is also affected by inhibition of NO. (Id.) Without being bound to any particular theory of mechanism, in certain embodiments the inventive gas-enriched fluids may be modulating localized and/or cellular NO production, or degradation, consistent with the spectrum of wound healing effects illustrated in the Examples section disclosed herein. Due to variable pathways of regulation, in certain embodiments, the inventive gas-enriched fluid may increase NO production and/or retard NO degradation, whereas in other certain embodiments, the inventive gas-enriched fluid may decrease NO production and/or hasten NO degradation.

Specifically, wounds treated with oxygen-enriched saline solution showed an increase in wound healing at days 4 through 11, and between days 3 and 11, the new epidermis in wounds treated with the oxygen-enriched saline solution migrated at two to four times as fast as the epidermis of the wounds treated with the normal saline solution, as set forth in Example 9 herein. The study also showed that between 15 and 22 days, wounds treated by the oxygen-enriched saline solution differentiated at a more rapid rate as evidenced by the earlier formation of more mature epidermal layers. At all stages, the thickening that occurs in the epidermis associated with normal healing did not occur within the wounds treated by the oxygen-enriched saline solution.

Thus, in accordance with this spectrum of wound healing effects, but without wishing to be bound by any particular theory, it is believed that the oxygen-enriched saline solution may modulate the localized and/or cellular level of NO within the wounds. NO modulates growth factors, collagen deposition, inflammation, mast cell migration, epidermal thickening, and neovascularization in wound healing. Furthermore, nitric oxide is produced by an inducible enzyme that is regulated by oxygen.

In the case of mast cell migration, differences also occurred in early and late migration for the oxygen-enriched solution. This is consistent with what is known in the art regarding inhibition of NO synthesis (Amadeu and Costa, J. Cutan Pathol 33: 465-473, 2006).

In the first two phases of the inflammatory process, the foreign body is either destroyed, for example, if the foreign body is an organism, or the tissue around it is loosened, for example, if it is a splinter. In the healing phase, the inflammation begins to subside; individual blood vessels and vascular patterns become normal once again; and repair of the wound commences. The three main events in the repair process are (1) formation of new connective tissue by proliferating fibroblasts; (2) regeneration of epithelium; and (3) outgrowth of new capillaries.

Even before the inflammation subsides, fibroblasts begin moving into the injured area from the surrounding normal tissue, where they usually exist in a dormant state. They migrate by an amoeboid movement along strands of fibrin and distribute themselves throughout the healing area. Once fixed into position in the injured tissue, they begin to synthesize collagen and secrete this protein, which arranges itself into fibers. The fibers orient themselves with their longitudinal axes in the direction of the greatest stress. As the collagen bundles grow in firmness, the fibroblasts gradually degenerate and attach closely to the bundles, and the injured area transforms into scar tissue.

Simultaneously with scar tissue formation, the intact epidermal cells on the edge of the wound begin to proliferate and move, as one sheet, toward the center of the injured area. As the inflammation subsides, a need for a direct supply of blood arises, and angiogenesis occurs at the wound site.

Inflammation is a complex process that involves multiple cell types. For example, mast cells release mediators that trigger an early phase of vasodilation, accompanied by the separation of endothelial cells and exposure of collagen fibers in the subendothelial layer. Fibers in the intercellular gaps that form in blood vessels trap platelets and trigger the release of mediators from these cells.

In addition to platelets, the exposed collagen fibers also interact with proteins of the plasma that filter through the pores of the dilated vessel wall, including the triggering factor of the blood-clotting cascade, increased vasodilation, increased blood vessel permeability, and chemotaxis.

Additionally, the complement cascade can be activated by several stimuli: the injured blood vessels, the proteolytic enzymes released by the damaged cells, the membrane components of any participating bacteria, and antigen-antibody complexes. Some of the activated complement components act as chemotactic factors, responsible for the influx of leukocytes into the inflamed area, while others facilitate phagocytosis and participate in cell lysis.

In addition, it is believed that the inventive gas-enriched fluids or solutions may also regulate at least one cytokine involved in at least one aspect of inflammation, the cytokine(s) including, but not limited to MAF (macrophage activating factor), MMIF (macrophage migration inhibition factor), MCF (macrophage chemotactic factor), LMIF (leukocyte migration inhibition factor), HRFs (histamine releasing factors), TF (transfer factors), interleukins (IL-1, IL-2, IL-3, IL-4, IL-5, IL-6, IL-7, IL-8, IL-9, IL-10, IL-11, IL-12, IL-13, IL-14, IL-15, etc.), TNF-α, TNF-β, interferons (IFN-α, IFN-β, IFN-γ, IFN-ζ, IFN-δ, etc.), G-CSF (granulocyte colony stimulating factor), GM-CSF (granulocyte-macrophage CSF), M-CSF (macrophage CSF), multi-CSF (IL-3), fibroblast growth factor (aFGF, bFGF), EGF (epidermal growth factor), NGF (nerve growth factor), PDGF (platelet-derived growth factor), VEGF (vascular endothelial growth factor), transforming growth factors (TGF-α, TGF-β, etc.), NAP-2 (neutrophil-activating protein 2), PF-4 (platelet factor 4), thromboglobulin, MCP-1 (monocyte chemoattractant protein 1), MCP-3, MIP-1α, MIP-1β-+ (macrophage inflammatory proteins), RANTES (regulated upon activation normal T expressed and presumably secreted chemokine), HSPs (heat shock proteins), GRPs (glucose-regulated proteins), ubiquitin, and others.

Thus, in certain embodiments, the gas-enriched fluids and/or therapeutic compositions may increase production and/or secretion of anti-inflammatory molecules or cytokines or decrease the degradation of anti-inflammatory molecules or cytokines, thereby alleviating or preventing at least one symptom of inflammation and/or inflammatory neurodegeneration. In other embodiments, the gas-enriched fluids and/or therapeutic compositions of the present invention may decrease production and/or secretion of pro-inflammatory molecules or cytokines or increase the degradation of pro-inflammatory molecules or cytokines, thereby alleviating or preventing at least one symptom of inflammation and/or inflammatory neurodegeneration.

Previous studies had shown a critical role of anti-MOG antibodies in augmentation of demyelination and worsening of EAE (experimental autoimmune encephalomyelitis), an animal model system for the human autoimmune disorder of rheumatoid arthritis. (Linington, et al. 1992. J. Neuroimmunol. 40:219-224). Additionally, antibodies against MOG have been implicated in the pathogenesis of multiple sclerosis. (Berger et al. N. Engl. J. Med. 2003 Jul. 10; 349(2):139-45).

As set forth in previous experiments the inventive gas-enriched fluid of the present invention amplifies the lymphocyte response to an antigen for which an animal was previously primed. As indicated in previous experiments, lymphocyte proliferation was greater for response to MOG challenge when cultured in fluid reconstituted with the inventive gas-enriched fluid comprising solvated electrons, when compared with pressurized, oxygenated fluid (pressure pot) or control deionized fluid.

### Exemplary Relevant Molecular Interactions:

Conventionally, quantum properties are thought to belong to elementary particles of less than 10⁻¹⁰ meters, while the macroscopic world of our everyday life is referred to as classical, in that it behaves according to Newton's laws of motion.

Recently, molecules have been described as forming clusters that increase in size with dilution. These clusters measure several micrometers in diameter, and have been reported to increase in size non-linearly with dilution. Quantum coherent domains measuring 100 nanometers in diameter have been postulated to arise in pure water, and collective vibrations of water molecules in the coherent domain may eventually become phase locked to electromagnetic field fluctuations, providing for stable oscillations in water, providing a form of ‘memory’ in the form of excitation of long lasting coherent oscillations specific to dissolved substances in the water that change the collective structure of the water, which may in turn determine the specific coherent oscillations that develop. Where these oscillations become stabilized by magnetic field phase coupling, the water, upon dilution may still carry ‘seed’ coherent oscillations. As a cluster of molecules increases in size, its electromagnetic signature is correspondingly amplified, reinforcing the coherent oscillations carried by the water.

Despite variations in the cluster size of dissolved molecules and detailed microscopic structure of the water, a specificity of coherent oscillations may nonetheless exist. One model for considering changes in properties of water is based on considerations involved in crystallization.

A simplified protonated water cluster forming a nanoscale cage is shown in Applicants' previous patent application: WO 2009/055729. A protonated water cluster typically takes the form of H⁺(H₂0)ₙ. Some protonated water clusters occur naturally, such as in the ionosphere. Without being bound by any particular theory, and according to particular aspects, other types of water clusters or structures (clusters, nanocages, etc) are possible, including structures comprising oxygen and stabilized electrons imparted to the inventive output materials. Oxygen atoms may be caught in the resulting structures. The chemistry of the semi-bound nanocage allows the oxygen and/or stabilized electrons to remain dissolved for extended periods of time. Other atoms or molecules, such as medicinal compounds, can be caged for sustained delivery purposes. The specific chemistry of the solution material and dissolved compounds depend on the interactions of those materials.

Fluids processed by the mixing device have been shown previously via experiments to exhibit different structural characteristics that are consistent with an analysis of the fluid in the context of a cluster structure. See, for example, WO 2009/055729.

### Charge-Stabilized Nanostructures (e.g., Charge Stabilized Oxygen-Containing Nanostructures):

As described previously in Applicants' WO 2009/055729, “Double Layer Effect,” “Dwell Time,” “Rate of Infusion,” and “Bubble size Measurements,” the electrokinetic mixing device creates, in a matter of milliseconds, a unique non-linear fluid dynamic interaction of the first material and the second material with complex, dynamic turbulence providing complex mixing in contact with an effectively enormous surface area (including those of the device and of the exceptionally small gas bubbles of less that 100 nm) that provides for the novel electrokinetic effects described herein. Additionally, feature-localized electrokinetic effects (voltage/current) were demonstrated using a specially designed mixing device comprising insulated rotor and stator features.

As well-recognized in the art, charge redistributions and/or solvated electrons are known to be highly unstable in aqueous solution. According to particular aspects, Applicants' electrokinetic effects (e.g., charge redistributions, including, in particular aspects, solvated electrons) are surprisingly stabilized within the output material (e.g., saline solutions, ionic solutions). In fact, as described herein, the stability of the properties and biological activity of the inventive electrokinetic fluids (e.g., RNS-60 or Solas) can be maintained for months in a gas-tight container, indicating involvement of dissolved gas (e.g., oxygen) in helping to generate and/or maintain, and/or mediate the properties and activities of the inventive solutions. Significantly, the charge redistributions and/or solvated electrons are stably configured in the inventive electrokinetic ionic aqueous fluids in an amount sufficient to provide, upon contact with a living cell (e.g., mammalian cell) by the fluid, modulation of at least one of cellular membrane potential and cellular membrane conductivity (see, e.g., cellular patch clamp working Example 23 from WO 2009/055729 and as disclosed herein).

As described herein under “Molecular Interactions,” to account for the stability and biological compatibility of the inventive electrokinetic fluids (e.g., electrokinetic saline solutions), Applicants have proposed that interactions between the water molecules and the molecules of the substances (e.g., oxygen) dissolved in the water change the collective structure of the water and provide for nanoscale cage clusters, including nanostructures comprising oxygen and/or stabilized electrons imparted to the inventive output materials. Without being bound by mechanism, the configuration of the nanostructures in particular aspects is such that they: comprise (at least for formation and/or stability and/or biological activity) dissolved gas (e.g., oxygen); enable the electrokinetic fluids (e.g., RNS-60 or Solas saline fluids) to modulate (e.g., impart or receive) charges and/or charge effects upon contact with a cell membrane or related constituent thereof; and in particular aspects provide for stabilization (e.g., carrying, harboring, trapping) solvated electrons in a biologically-relevant form.

According to particular aspects, and as supported by the present disclosure, in ionic or saline (e.g., standard saline, NaCl) solutions, the inventive nanostructures comprise charge stabilized nanostrutures (e.g., average diameter less that 100 nm) that may comprise at least one dissolved gas molecule (e.g., oxygen) within a charge-stabilized hydration shell. According to additional aspects, the charge-stabilized hydration shell may comprise a cage or void harboring the at least one dissolved gas molecule (e.g., oxygen). According to further aspects, by virtue of the provision of suitable charge-stabilized hydration shells, the charge-stabilized nanostructure and/or charge-stabilized oxygen containing nano-structures may additionally comprise a solvated electron (e.g., stabilized solvated electron).

Without being bound by mechanism or particular theory, after the present priority date, charge-stabilized microbubbles stabilized by ions in aqueous liquid in equilibrium with ambient (atmospheric) gas have been proposed (Bunkin et al., Journal of Experimental and Theoretical Physics, 104:486-498, 2007; incorporated herein by reference in its entirety). According to particular aspects of the present invention, Applicants' novel electrokinetic fluids comprise a novel, biologically active form of charge-stabilized oxygen-containing nanostructures, and may further comprise novel arrays, clusters or associations of such structures.

According to the charge-stabilized microbubble model, the short-range molecular order of the water structure is destroyed by the presence of a gas molecule (e.g., a dissolved gas molecule initially complexed with a nonadsorptive ion provides a short-range order defect), providing for condensation of ionic droplets, wherein the defect is surrounded by first and second coordination spheres of water molecules, which are alternately filled by adsorptive ions (e.g., acquisition of a ‘screening shell of Na⁻ ions to form an electrical double layer) and nonadsorptive ions (e.g., Cl⁻ ions occupying the second coordination sphere) occupying six and 12 vacancies, respectively, in the coordination spheres. In under-saturated ionic solutions (e.g., undersaturated saline solutions), this hydrated ‘nucleus’ remains stable until the first and second spheres are filled by six adsorptive and five nonadsorptive ions, respectively, and then undergoes Coulomb explosion creating an internal void containing the gas molecule, wherein the adsorptive ions (e.g., Na⁺ ions) are adsorbed to the surface of the resulting void, while the nonadsorptive ions (or some portion thereof) diffuse into the solution (Bunkin et al., supra). In this model, the void in the nanostructure is prevented from collapsing by Coulombic repulsion between the ions (e.g., Na⁺ ions) adsorbed to its surface. The stability of the void-containing nanostrutures is postulated to be due to the selective adsorption of dissolved ions with like charges onto the void/bubble surface and diffusive equilibrium between the dissolved gas and the gas inside the bubble, where the negative (outward electrostatic pressure exerted by the resulting electrical double layer provides stable compensation for surface tension, and the gas pressure inside the bubble is balanced by the ambient pressure. According to the model, formation of such microbubbles requires an ionic component, and in certain aspects collision-mediated associations between particles may provide for formation of larger order clusters (arrays) (Id).

The charge-stabilized microbubble model suggests that the particles can be gas microbubbles, but contemplates only spontaneous formation of such structures in ionic solution in equilibrium with ambient air, is uncharacterized and silent as to whether oxygen is capable of forming such structures, and is likewise silent as to whether solvated electrons might be associated and/or stabilized by such structures.

According to particular aspects, the inventive electrokinetic fluids comprising charge-stabilized nanostructures and/or charge-stabilized oxygen-containing nanostructures are novel and fundamentally distinct from the postulated non-electrokinetic, atmospheric charge-stabilized microbubble structures according to the microbubble model. Significantly, this conclusion is unavoidable, deriving, at least in part, from the fact that control saline solutions do not have the biological properties disclosed herein, whereas Applicants' charge-stabilized nanostructures provide a novel, biologically active form of charge-stabilized oxygen-containing nanostructures.

According to particular aspects of the present invention, Applicants' novel electrokinetic device and methods provide for novel electrokinetically-altered fluids comprising significant quantities of charge-stabilized nanostructures in excess of any amount that may or may not spontaneously occur in ionic fluids in equilibrium with air, or in any non-electrokinetically generated fluids. In particular aspects, the charge-stabilized nanostructures comprise charge-stabilized oxygen-containing nanostructures. In additional aspects, the charge-stabilized nanostrutures are all, or substantially all charge-stabilized oxygen-containing nanostructures, or the charge-stabilized oxygen-containing nanostructures the major charge-stabilized gas-containing nanostructure species in the electrokinetic fluid.

According to yet further aspects, the charge-stabilized nanostructures and/or the charge-stabilized oxygen-containing nanostructures may comprise or harbor a solvated electron, and thereby provide a novel stabilized solvated electron carrier. In particular aspects, the charge-stabilized nanostructures and/or the charge-stabilized oxygen-containing nanostructures provide a novel type of electride (or inverted electride), which in contrast to conventional solute electrides having a single organically coordinated cation, rather have a plurality of cations stably arrayed about a void or a void containing an oxygen atom, wherein the arrayed sodium ions are coordinated by water hydration shells, rather than by organic molecules. According to particular aspects, a solvated electron may be accommodated by the hydration shell of water molecules, or preferably accommodated within the nanostructure void distributed over all the cations. In certain aspects, the inventive nanostructures provide a novel ‘super electride’ structure in solution by not only providing for distribution/stabilization of the solvated electron over multiple arrayed sodium cations, but also providing for association or partial association of the solvated electron with the caged oxygen molecule(s) in the void—the solvated electron distributing over an array of sodium atoms and at least one oxygen atom. According to particular aspects, therefore, ‘solvated electrons’ as presently disclosed in association with the inventive electrokinetic fluids, may not be solvated in the traditional model comprising direct hydration by water molecules. Alternatively, in limited analogy with dried electride salts, solvated electrons in the inventive electrokinetic fluids may be distributed over multiple charge-stabilized nanostructures to provide a ‘lattice glue’ to stabilize higher order arrays in aqueous solution.

In particular aspects, the inventive charge-stabilized nanostructures and/or the charge-stabilized oxygen-containing nanostructures are capable of interacting with cellular membranes or constituents thereof, or proteins, etc., to mediate biological activities. In particular aspects, the inventive charge-stabilized nanostructures and/or the charge-stabilized oxygen-containing nanostructures harboring a solvated electron are capable of interacting with cellular membranes or constituents thereof, or proteins, etc., to mediate biological activities.

In particular aspects, the inventive charge-stabilized nanostructures and/or the charge-stabilized oxygen-containing nanostructures interact with cellular membranes or constituents thereof, or proteins, etc., as a charge and/or charge effect donor (delivery) and/or as a charge and/or charge effect recipient to mediate biological activities. In particular aspects, the inventive charge-stabilized nanostructures and/or the charge-stabilized oxygen-containing nanostructures harboring a solvated electron interact with cellular membranes as a charge and/or charge effect donor and/or as a charge and/or charge effect recipient to mediate biological activities.

In particular aspects, the inventive charge-stabilized nanostructures and/or the charge-stabilized oxygen-containing nanostructures are consistent with, and account for the observed stability and biological properties of the inventive electrokinetic fluids, and further provide a novel electride (or inverted electride) that provides for stabilized solvated electrons in aqueous ionic solutions (e.g., saline solutions, NaCl, etc.).

In particular aspects, the charge-stabilized oxygen-containing nanostructures substantially comprise, take the form of, or can give rise to, charge-stabilized oxygen-containing nanobubbles. In particular aspects, charge-stabilized oxygen-containing clusters provide for formation of relatively larger arrays of charge-stabilized oxygen-containing nanostructures, and/or charge-stabilized oxygen-containing nanobubbles or arrays thereof. In particular aspects, the charge-stabilized oxygen-containing nanostructures can provide for formation of hydrophobic nanobubbles upon contact with a hydrophobic surface.

In particular aspects, the charge-stabilized oxygen-containing nanostructures substantially comprise at least one oxygen molecule. In certain aspects, the charge-stabilized oxygen-containing nanostructures substantially comprise at least 1, at least 2, at least 3, at least 4, at least 5, at least 10 at least 15, at least 20, at least 50, at least 100, or greater oxygen molecules. In particular aspects, charge-stabilized oxygen-containing nanostructures comprise or give rise to nanobubles (e.g., hydrophobid nanobubbles) of about 20 nm×1.5 nm, comprise about 12 oxygen molecules (e.g., based on the size of an oxygen molecule (approx 0.3 nm by 0.4 nm), assumption of an ideal gas and application of n=PV/RT, where P=1 atm, R=0.082 057 Latm/mol.K; T=295K; V=pr²h=4.7×10⁻²² L, where r=10×10⁻⁹ m, h=1.5×10⁻⁹ m, and n=1.95×10⁻²² moles).

In certain aspects, the percentage of oxygen molecules present in the fluid that are in such nanostructures, or arrays thereof, having a charge-stabilized configuration in the ionic aqueous fluid is a percentage amount selected from the group consisting of greater than: 0.1%, 1%; 2%; 5%; 10%; 15%; 20%; 25%; 30%; 35%; 40%; 45%; 50%; 55%; 60%; 65%; 70%; 75%; 80%; 85%; 90%; and greater than 95%. Preferably, this percentage is greater than about 5%, greater than about 10%, greater than about 15%f, or greater than about 20%. In additional aspects, the substantial size of the charge-stabilized oxygen-containing nanostructures, or arrays thereof, having a charge-stabilized configuration in the ionic aqueous fluid is a size selected from the group consisting of less than: 100 nm; 90 nm; 80 nm; 70 nm; 60 nm; 50 nm; 40 nm; 30 nm; 20 nm; 10 nm; 5 nm; 4 nm; 3 nm; 2 nm; and 1 nm. Preferably, this size is less than about 50 nm, less than about 40 nm, less than about 30 nm, less than about 20 nm, or less than about 10 nm.

In certain aspects, the inventive electrokinetic fluids comprise solvated electrons. In further aspects, the inventive electrokinetic fluids comprises charge-stabilized nanostructures and/or charge-stabilized oxygen-containing nanostructures, and/or arrays thereof, which comprise at least one of: solvated electron(s); and unique charge distributions (polar, symmetric, asymmetric charge distribution). In certain aspects, the charge-stabilized nanostructures and/or charge-stabilized oxygen-containing nanostructures, and/or arrays thereof, have paramagnetic properties.

By contrast, relative to the inventive electrokinetic fluids, control pressure pot oxygenated fluids (non-electrokinetic fluids) and the like do not comprise such electrokinetically generated charge-stabilized biologically-active nanostructures and/or biologically-active charge-stabilized oxygen-containing nanostructures and/or arrays thereof, capable of modulation of at least one of cellular membrane potential and cellular membrane conductivity.

### Systems for Making Gas-Enriched Fluids

The system and methods as previously disclosed in Applicants' WO 2009/055729 patent application allow gas (e.g. oxygen) to be enriched stably at a high concentration with minimal passive loss. This system and methods can be effectively used to enrich a wide variety of gases at heightened percentages into a wide variety of fluids. By way of example only, deionized water at room temperature that typically has levels of about 2-3 ppm (parts per million) of dissolved oxygen can achieve levels of dissolved oxygen ranging from at least about 5 ppm, at least about 10 ppm, at least about 15 ppm, at least about 20 ppm, at least about 25 ppm, at least about 30 ppm, at least about 35 ppm, at least about 40 ppm, at least about 45 ppm, at least about 50 ppm, at least about 55 ppm, at least about 60 ppm, at least about 65 ppm, at least about 70 ppm, at least about 75 ppm, at least about 80 ppm, at least about 85 ppm, at least about 90 ppm, at least about 95 ppm, at least about 100 ppm, or any value greater or therebetween using the disclosed systems and/or methods. In accordance with a particular exemplary embodiment, oxygen-enriched water may be generated with levels of about 30-60 ppm of dissolved oxygen.

Table 3 illustrates various partial pressure measurements taken in a healing wound treated with an oxygen-enriched saline solution (Table 3) and in samples of the gas-enriched oxygen-enriched saline solution of the present invention.

TABLE 3
TISSUE OXYGEN MEASUREMENTS
Probe Z082BO
In air: 171 mmHg 23° C.

| Column | Partial Pressure (mmHg) |
|---|---|
| B1 | 32-36 |
| B2 | 169-200 |
| B3 | 20-180* |
| B4 | 40-60 |

*wound depth minimal, majority >150, occasional 20 s

Routes and Forms of Administration

In particular exemplary embodiments, the gas-enriched fluid of the present invention may function as a therapeutic composition alone or in combination with another therapeutic agent such that the therapeutic composition prevents or alleviates at least one symptom of inflammation. The therapeutic compositions of the present invention include compositions that are able to be administered to a subject in need thereof. In certain embodiments, the therapeutic composition formulation may also comprise at least one additional agent selected from the group consisting of: carriers, adjuvants, emulsifying agents, suspending agents, sweeteners, flavorings, perfumes, and binding agents.

As used herein, “pharmaceutically acceptable carrier” and “carrier” generally refer to a non-toxic, inert solid, semi-solid or liquid filler, diluent, encapsulating material or formulation auxiliary of any type. Some non-limiting examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. In particular aspects, such carriers and excipients may be gas-enriched fluids or solutions of the present invention.

The pharmaceutically acceptable carriers described herein, for example, vehicles, adjuvants, excipients, or diluents, are well known to those who are skilled in the art. Typically, the pharmaceutically acceptable carrier is chemically inert to the therapeutic agents and has no detrimental side effects or toxicity under the conditions of use. The pharmaceutically acceptable carriers can include polymers and polymer matrices, nanoparticles, microbubbles, and the like.

In addition to the therapeutic gas-enriched fluid of the present invention, the therapeutic composition may further comprise inert diluents such as additional non-gas-enriched water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. As is appreciated by those of ordinary skill, a novel and improved formulation of a particular therapeutic composition, a novel gas-enriched therapeutic fluid, and a novel method of delivering the novel gas-enriched therapeutic fluid may be obtained by replacing one or more inert diluents with a gas-enriched fluid of identical, similar, or different composition. For example, conventional water may be replaced or supplemented by a gas-enriched fluid produced by mixing oxygen into water or deionized water to provide gas-enriched fluid.

In certain embodiments, the inventive gas-enriched fluid may be combined with one or more therapeutic agents and/or used alone. In particular embodiments, incorporating the gas-enriched fluid may include replacing one or more solutions known in the art, such as deionized water, saline solution, and the like with one or more gas-enriched fluid, thereby providing an improved therapeutic composition for delivery to the subject.

Certain embodiments provide for therapeutic compositions comprising a gas-enriched fluid of the present invention, a pharmaceutical composition or other therapeutic agent or a pharmaceutically acceptable salt or solvate thereof, and at least one pharmaceutical carrier or diluent. These pharmaceutical compositions may be used in the prophylaxis and treatment of the foregoing diseases or conditions and in therapies as mentioned above. Preferably, the carrier must be pharmaceutically acceptable and must be compatible with, i.e. not have a deleterious effect upon, the other ingredients in the composition. The carrier may be a solid or liquid and is preferably formulated as a unit dose formulation, for example, a tablet that may contain from 0.05 to 95% by weight of the active ingredient.

Possible administration routes include oral, sublingual, buccal, parenteral (for example subcutaneous, intramuscular, intra-arterial, intraperitoneally, intracisternally, intravesically, intrathecally, or intravenous), rectal, topical including transdermal, intravaginal, intraoccular, intraotical, intranasal, inhalation, and injection or insertion of implantable devices or materials.

### Administration Routes

Most suitable means of administration for a particular subject will depend on the nature and severity of the disease or condition being treated or the nature of the therapy being used, as well as the nature of the therapeutic composition or additional therapeutic agent. In certain embodiments, oral or topical administration is preferred.

Formulations suitable for oral administration may be provided as discrete units, such as tablets, capsules, cachets, syrups, elixirs, chewing gum, “lollipop” formulations, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

Additional formulations suitable for oral administration may be provided to include fine particle dusts or mists which may be generated by means of various types of metered dose pressurized aerosols, atomizers, nebulisers, or insufflators. In particular, powders or other compounds of therapeutic agents may be dissolved or suspended in a gas-enriched fluid of the present invention.

Formulations suitable for transmucosal methods, such as by sublingual or buccal administration include lozenges patches, tablets, and the like comprising the active compound and, typically a flavored base, such as sugar and acacia or tragacanth and pastilles comprising the active compound in an inert base, such as gelatin and glycerine or sucrose acacia.

Formulations suitable for parenteral administration typically comprise sterile aqueous solutions containing a predetermined concentration of the active gas-enriched fluid and possibly another therapeutic agent; the solution is preferably isotonic with the blood of the intended recipient. Additional formulations suitable for parenteral administration include formulations containing physiologically suitable co-solvents and/or complexing agents such as surfactants and cyclodextrins. Oil-in-water emulsions may also be suitable for formulations for parenteral administration of the gas-enriched fluid. Although such solutions are preferably administered intravenously, they may also be administered by subcutaneous or intramuscular injection.

Formulations suitable for urethral, rectal or vaginal administration include gels, creams, lotions, aqueous or oily suspensions, dispersible powders or granules, emulsions, dissolvable solid materials, douches, and the like. The formulations are preferably provided as unit-dose suppositories comprising the active ingredient in one or more solid carriers forming the suppository base, for example, cocoa butter. Alternatively, colonic washes with the gas-enriched fluids of the present invention may be formulated for colonic or rectal administration.

Formulations suitable for topical, intraoccular, intraotic, or intranasal application include ointments, creams, pastes, lotions, pastes, gels (such as hydrogels), sprays, dispersible powders and granules, emulsions, sprays or aerosols using flowing propellants (such as liposomal sprays, nasal drops, nasal sprays, and the like) and oils. Suitable carriers for such formulations include petroleum jelly, lanolin, polyethyleneglycols, alcohols, and combinations thereof. Nasal or intranasal delivery may include metered doses of any of these formulations or others. Likewise, intraotic or intraocular may include drops, ointments, irritation fluids and the like.

Formulations of the invention may be prepared by any suitable method, typically by uniformly and intimately admixing the gas-enriched fluid optionally with an active compound with liquids or finely divided solid carriers or both, in the required proportions and then, if necessary, shaping the resulting mixture into the desired shape.

For example a tablet may be prepared by compressing an intimate mixture comprising a powder or granules of the active ingredient and one or more optional ingredients, such as a binder, lubricant, inert diluent, or surface active dispersing agent, or by molding an intimate mixture of powdered active ingredient and a gas-enriched fluid of the present invention.

Suitable formulations for administration by inhalation include fine particle dusts or mists which may be generated by means of various types of metered dose pressurized aerosols, atomizers, nebulisers, or insufflators. In particular, powders or other compounds of therapeutic agents may be dissolved or suspended in a gas-enriched fluid of the present invention.

For pulmonary administration via the mouth, the particle size of the powder or droplets is typically in the range 0.5-10 μM, preferably 1-5 μM,to ensure delivery into the bronchial tree. For nasal administration, a particle size in the range 10-500 μM is preferred to ensure retention in the nasal cavity.

Metered dose inhalers are pressurized aerosol dispensers, typically containing a suspension or solution formulation of a therapeutic agent in a liquefied propellant. In certain embodiments, as disclosed herein, the gas-enriched fluids of the present invention may be used in addition to or instead of the standard liquefied propellant. During use, these devices discharge the formulation through a valve adapted to deliver a metered volume, typically from 10 to 150 μL, to produce a fine particle spray containing the therapeutic agent and the gas-enriched fluid. Suitable propellants include certain chlorofluorocarbon compounds, for example, dichlorodifluoromethane, trichlorofluoromethane, dichlorotetrafluoroethane and mixtures thereof.

The formulation may additionally contain one or more co-solvents, for example, ethanol surfactants, such as oleic acid or sorbitan trioleate, anti-oxidants and suitable flavoring agents. Nebulisers are commercially available devices that transform solutions or suspensions of the active ingredient into a therapeutic aerosol mist either by means of acceleration of a compressed gas (typically air or oxygen) through a narrow venturi orifice, or by means of ultrasonic agitation. Suitable formulations for use in nebulisers consist of another therapeutic agent in a gas-enriched fluid and comprising up to 40% w/w of the formulation, preferably less than 20% w/w. In addition, other carriers may be utilized, such as distilled water, sterile water, or a dilute aqueous alcohol solution, preferably made isotonic with body fluids by the addition of salts, such as sodium chloride. Optional additives include preservatives, especially if the formulation is not prepared sterile, and may include methyl hydroxy-benzoate, anti-oxidants, flavoring agents, volatile oils, buffering agents and surfactants.

Suitable formulations for administration by insufflation include finely comminuted powders that may be delivered by means of an insufflator or taken into the nasal cavity in the manner of a snuff. In the insufflator, the powder is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation.

In addition to the ingredients specifically mentioned above, the formulations of the present invention may include other agents known to those skilled in the art, having regard for the type of formulation in issue. For example, formulations suitable for oral administration may include flavoring agents and formulations suitable for intranasal administration may include perfumes.

The therapeutic compositions of the invention can be administered by any conventional method available for use in conjunction with pharmaceutical drugs, either as individual therapeutic agents or in a combination of therapeutic agents.

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to 1000 milligrams (mg) per kilogram (kg) of body weight, with the preferred dose being 0.1 to about 30 mg/kg. According to certain aspects daily dosage of active ingredient may be 0.001 liters to 10 liters, with the preferred dose being from about 0.01 liters to 1 liter.

Dosage forms (compositions suitable for administration) contain from about 1 mg to about 500 mg of active ingredient per unit. In these pharmaceutical compositions, the active ingredient will ordinarily be present in an amount of about 0.5-95% weight based on the total weight of the composition.

Ointments, pastes, foams, occlusions, creams and gels also can contain excipients, such as starch, tragacanth, cellulose derivatives, silicones, bentonites, silica acid, and talc, or mixtures thereof. Powders and sprays also can contain excipients such as lactose, talc, silica acid, aluminum hydroxide, and calcium silicates, or mixtures of these substances. Solutions of nanocrystalline antimicrobial metals can be converted into aerosols or sprays by any of the known means routinely used for making aerosol pharmaceuticals. In general, such methods comprise pressurizing or providing a means for pressurizing a container of the solution, usually with an inert carrier gas, and passing the pressurized gas through a small orifice. Sprays can additionally contain customary propellants, such as nitrogen, carbon dioxide, and other inert gases. In addition, microspheres or nanoparticles may be employed with the gas-enriched therapeutic compositions or fluids of the present invention in any of the routes required to administer the therapeutic compounds to a subject.

The injection-use formulations can be presented in unit-dose or multi-dose sealed containers, such as ampules and vials, and can be stored in a freeze-dried (lyophilized) condition requiring only the addition of the sterile liquid excipient, or gas-enriched fluid, immediately prior to use. Extemporaneous injection solutions and suspensions can be prepared from sterile powders, granules, and tablets. The requirements for effective pharmaceutical carriers for injectable compositions are well known to those of ordinary skill in the art. See, for example, Pharmaceutics and Pharmacy Practice, J. B. Lippincott Co., Philadelphia, Pa., Banker and Chalmers, Eds., 238-250 (1982) and ASHP Handbook on Injectable Drugs, Toissel, 4th ed., 622-630 (1986).

Formulations suitable for topical administration include lozenges comprising a gas-enriched fluid of the invention and optionally, an additional therapeutic and a flavor, usually sucrose and acacia or tragacanth; pastilles comprising a gas-enriched fluid and optional additional therapeutic agent in an inert base, such as gelatin and glycerin, or sucrose and acacia; and mouth washes or oral rinses comprising a gas-enriched fluid and optional additional therapeutic agent in a suitable liquid carrier; as well as creams, emulsions, gels and the like.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable pharmaceutical carriers are described in Remington's Pharmaceutical Sciences, Mack Publishing Company, a standard reference text in this field.

The dose administered to a subject, especially an animal, particularly a human, in the context of the present invention should be sufficient to affect a therapeutic response in the animal over a reasonable time frame. One skilled in the art will recognize that dosage will depend upon a variety of factors including the condition of the animal, the body weight of the animal, as well as the condition being treated. A suitable dose is that which will result in a concentration of the therapeutic composition in a subject that is known to affect the desired response.

The size of the dose also will be determined by the route, timing and frequency of administration as well as the existence, nature, and extent of any adverse side effects that might accompany the administration of the therapeutic composition and the desired physiological effect.

It will be appreciated that the compounds of the combination may be administered: (1) simultaneously by combination of the compounds in a co-formulation or (2) by alternation, i.e. delivering the compounds serially, sequentially, in parallel or simultaneously in separate pharmaceutical formulations. In alternation therapy, the delay in administering the second, and optionally a third active ingredient, should not be such as to lose the benefit of a synergistic therapeutic effect of the combination of the active ingredients. According to certain embodiments by either method of administration (1) or (2), ideally the combination should be administered to achieve the most efficacious results. In certain embodiments by either method of administration (1) or (2), ideally the combination should be administered to achieve peak plasma concentrations of each of the active ingredients. A one pill once-per-day regimen by administration of a combination co-formulation may be feasible for some patients expected to be exposed to a neurotoxin. According to certain embodiments effective peak plasma concentrations of the active ingredients of the combination will be in the range of approximately 0.001 to 100 μM. Optimal peak plasma concentrations may be achieved by a formulation and dosing regimen prescribed for a particular patient. It will also be understood that the inventive fluids and any one of erythropoietin, anti-apoptotics (TCH346, CEP-1347), antiglutamatergics, monoamine oxidase inhibitors (selegiline, rasagiline), promitochondrials (coenzyme Q10, creatine), calcium channel blockers (isradipine), alpha-synuclein, and/or growth factors (GDNF) or the physiologically functional derivatives of any thereof, whether presented simultaneously or sequentially, may be administered individually, in multiples, or in any combination thereof. In general, during alternation therapy (2), an effective dosage of each compound is administered serially, where in co-formulation therapy (1), effective dosages of two or more compounds are administered together.

The combinations of the invention may conveniently be presented as a pharmaceutical formulation in a unitary dosage form. A convenient unitary dosage formulation contains the active ingredients in any amount from 1 mg to 1 g each, for example but not limited to, 10 mg to 300 mg. The synergistic effects of the inventive fluid in combination with any one of erythropoietin, anti-apoptotics (TCH346, CEP-1347), antiglutamatergics, monoamine oxidase inhibitors (selegiline, rasagiline), promitochondrials (coenzyme Q10, creatine), calcium channel blockers (isradipine), alpha-synuclein, and/or growth factors (GDNF) may be realized over a wide ratio, for example 1:50 to 50:1 (inventive fluid: erythropoietin, anti-apoptotics (TCH346, CEP-1347), antiglutamatergics, monoamine oxidase inhibitors (selegiline, rasagiline), promitochondrials (coenzyme Q10, creatine), calcium channel blockers (isradipine), alpha-synuclein, and/or growth factors (GDNF)). In one embodiment the ratio may range from about 1:10 to 10:1. In another embodiment, the weight/weight ratio of inventive fluid to any one of erythropoietin, anti-apoptotics (TCH346, CEP-1347), antiglutamatergics, monoamine oxidase inhibitors (selegiline, rasagiline), promitochondrials (coenzyme Q10, creatine), calcium channel blockers (isradipine), alpha-synuclein, and/or growth factors (GDNF) in a co-formulated combination dosage form, such as a pill, tablet, caplet or capsule will be about 1, i.e. an approximately equal amount of inventive fluid and any one of erythropoietin, anti-apoptotics (TCH346, CEP-1347), antiglutamatergics, monoamine oxidase inhibitors (selegiline, rasagiline), promitochondrials (coenzyme Q10, creatine), calcium channel blockers (isradipine), alpha-synuclein, and/or growth factors (GDNF). In other exemplary co-formulations, there may be more or less inventive fluid and any one of erythropoietin, anti-apoptotics (TCH346, CEP-1347), antiglutamatergics, monoamine oxidase inhibitors (selegiline, rasagiline), promitochondrials (coenzyme Q10, creatine), calcium channel blockers (isradipine), alpha-synuclein, and/or growth factors (GDNF). In one embodiment, each compound will be employed in the combination in an amount at which it exhibits anti-inflammatory activity when used alone. Other ratios and amounts of the compounds of said combinations are contemplated within the scope of the invention.

A unitary dosage form may further comprise inventive fluid and any one of erythropoietin, anti-apoptotics (TCH346, CEP-1347), antiglutamatergics, monoamine oxidase inhibitors (selegiline, rasagiline), promitochondrials (coenzyme Q10, creatine), calcium channel blockers (isradipine), alpha-synuclein, and/or growth factors (GDNF), or physiologically functional derivatives of either thereof, and a pharmaceutically acceptable carrier.

It will be appreciated by those skilled in the art that the amount of active ingredients in the combinations of the invention required for use in treatment will vary according to a variety of factors, including the nature of the condition being treated and the age and condition of the patient, and will ultimately be at the discretion of the attending physician or health care practitioner. The factors to be considered include the route of administration and nature of the formulation, the animal's body weight, age and general condition and the nature and severity of the disease to be treated.

It is also possible to combine any two of the active ingredients in a unitary dosage form for simultaneous or sequential administration with a third active ingredient. The three-part combination may be administered simultaneously or sequentially. When administered sequentially, the combination may be administered in two or three administrations. According to certain embodiments the three-part combination of inventive fluid and any one of erythropoietin, anti-apoptotics (TCH346, CEP-1347), antiglutamatergics, monoamine oxidase inhibitors (selegiline, rasagiline), promitochondrials (coenzyme Q10, creatine), calcium channel blockers (isradipine), alpha-synuclein, and/or growth factors (GDNF) may be administered in any order.

### Neurotoxic Agents:

Neurotoxic agents are toxins that specifically act upon neurons, their synapses, or the nervous system in its entirety. They are substances which cause damage to the structures of the brain which in turn leads to chronic disease. Neurotoxins include adrenergic neurotoxins, cholinergic neurotoxins, dopaminergic neurotoxins, excitotoxins, and other neurotoxins. Examples of adrenergic neurotoxins include N-(2-chloroethyl)-N-ethyl-2-bromobenzylamine hydrochloride. Examples of cholinergic neurotoxins include acetylethylcholine mustard hydrochloride. Examples of dopaminergic neurotoxins include 6-hydroxydopamine HBr (6-OHDA), 1-methyl-4-(2-methylphenyl)-1,2,3,6-tetrahydro-pyridine hydrochloride, 1-methyl-4-phenyl-2,3-dihydropyridinium perchlorate, N-methyl-4-phenyl-1,2,5,6tetrahydropyridine HCl (MPTP), 1-methyl-4-phenylpyridinium iodide (MPP+), paraquat, and rotenone. Examples of excitotoxins include NMDA and kainic acid.

MPTP, MPP+, paraquat, rotenone and 6-OHDA have been been shown to induce PD like symptoms in animal models. (See, K. Ossowska, et al., (2006). “Degeneration of dopaminergic mesocortical neurons and activation of compensatory processes induced by a long-term paraquat administration in rats: Implications for Parkinson's disease”. Neuroscience 141 (4): 2155-2165; and Caboni P, et al., (2004). “Rotenone, deguelin, their metabolites, and the rat model of Parkinson's disease”. Chem Res Toxicol 17 (11): 1540-8; Simon et al., Exp Brain Res, 1974, 20: 375-384; Langston et al., Science, 1983, 219: 979-980; Tanner, Occup Med, 1992, 7: 503-513; Liou et al., Neurology, 1997, 48: 1583-1588).

### Neuroprotective

Neuroprotection within the nervous system protects neurons from apoptosis or degeneration, for example following a brain injury or as a result of chronic neurodegenerative diseases. A “neuroprotective effect” is aimed to prevent and treat complications that might result in central nervous system (CNS) damage. Neuroprotection can be estimated by parameters of cell survival or cell death delay, arrest or slowing of the disease progression, disease onset and disease mortality delay.

Examples, as described herein, show that the electrokinetically altered aqueous fluids have neuro protective properties, wherein the electrokinetically altered aqueous fluids was shown to protect neurocells from MPTP-induced PD symptoms. According to certain embodiments, the electrokinetically altered aqueous fluids have substantial utility in protecting against and/or reducing the effects related to being exposed to neurotoxins.

Neuroprotective agents include but are not limited to erythropoietin, anti-apoptotics (TCH346, CEP-1347), antiglutamatergics, monoamine oxidase inhibitors (selegiline, rasagiline), promitochondrials (coenzyme Q10, creatine), calcium channel blockers (isradipine), alpha-synuclein, and growth factors (GDNF).

The following examples are meant to be illustrative only and not limiting in any way.

### EXAMPLES

### Example 1

### Microbubble Size

Experiments were performed with a gas-enriched fluid by using the diffuser of the present invention in order to determine a gas microbubble size limit. The microbubble size limit was established by passing the gas enriched fluid through 0.22 and 0.1 micron filters. In performing these tests, a volume of fluid passed through the diffuser of the present invention and generated a gas-enriched fluid. Sixty milliliters of this fluid was drained into a 60 ml syringe. The dissolved oxygen level of the fluid within the syringe was then measured by Winkler titration. The fluid within the syringe was injected through a 0.22 micron Millipore Millex GP50 filter and into a 50 ml beaker. The dissolved oxygen rate of the material in the 50 ml beaker was then measured. The experiment was performed three times to achieve the results illustrated in Table 4 below.

TABLE 4

| | DO AFTER 0.22 MICRON |
|---|---|
| DO IN SYRINGE | FILTER |
| 42.1 ppm | 39.7 ppm |
| 43.4 ppm | 42.0 ppm |
| 43.5 ppm | 39.5 ppm |

As can be seen, the dissolved oxygen levels that were measured within the syringe and the dissolved oxygen levels within the 50 ml beaker were not significantly changed by passing the diffused material through a 0.22 micron filter, which implies that the microbubbles of dissolved gas within the fluid are not larger than 0.22 microns.

A second test was performed in which a batch of saline solution was enriched with the diffuser of the present invention and a sample of the output solution was collected in an unfiltered state. The dissolved oxygen level of the unfiltered sample was 44.7 ppm. A 0.1 micron filter was used to filter the oxygen-enriched solution from the diffuser of the present invention and two additional samples were taken. For the first sample, the dissolved oxygen level was 43.4 ppm. For the second sample, the dissolved oxygen level was 41.4 ppm. Finally, the filter was removed and a final sample was taken from the unfiltered solution. In this case, the final sample had a dissolved oxygen level of 45.4 ppm. These results were consistent with those in which the Millipore 0.22 micron filter was used. Thus, the majority of the gas bubbles or microbubbles within the saline solution are approximately less than 0.1 microns in size.

### Example 2

(Patch Clamp Analysis Conducted on Calu-3 Cells Perfused with Inventive Electrokinetically Generated Fluids (RNS-60 and Solas) Revealed that (i) Exposure to RNS-60 and Solas Resulted in Increases in Whole Cell Conductance, (it) that Exposure of Cells to the RNS-60 Produced an Increase in a Non-Linear Conductance, Evident at 15 min Incubation Times, and (iii) that Exposure of Cells to the RNS-60 Produced an Effect of RNS-60 Saline on Calcium Permeable Channels)

Overview. In this Example, patch clamp studies were performed to further confirm the utilities, as described herein, of the inventive electrokinetically generated saline fluids (RNS-60 and Solas), including the utility to modulate whole-cell currents. Two sets of experiments were conducted.

The summary of the data of the first set of experiments indicates that the whole cell conductance (current-to-voltage relationship) obtained with Solas saline is highly linear for both incubation times (15 min, 2 hours), and for all voltage protocols. It is however evident, that longer incubation (2 hours) with Solas increased the whole cell conductance. Exposure of cells to the RNS-60 produced an increase in a non-linear conductance, as shown in the delta currents (Rev-Sol subtraction), which is only evident at 15 min incubation time. The effect of the RNS-60 on this non-linear current disappears, and is instead highly linear at the two-hour incubation time. The contribution of the non-linear whole cell conductance, as previously observed, was voltage sensitive, although present at all voltage protocols.

The summary of data of the second set of experiments indicates that there is an effect of the RNS-60 saline on a non-linear current, which was made evident in high calcium in the external solution. The contribution of the non-linear whole cell conductance, although voltage sensitive, was present in both voltage protocols, and indicates an effect of RNS-60 saline on calcium permeable channels.

### First Set of Experiments (Increase of Conductance; and Activation of a Non-Linear Voltage Regulated Conductance)

### Materials and Methods:

The Bronchial Epithelial line Calu-3 was used in Patch clamp studies. Calu-3 Bronchial Epithelial cells (ATCC #HTB-55) were grown in a 1:1 mixture of Ham's F12 and DMEM medium that was supplemented with 10% FBS onto glass coverslips until the time of the experiments. In brief, a whole cell voltage clamp device was used to measure effects on Calu-3 cells exposed to the inventive electrokinetically generated fluids (e.g., RNS-60; electrokinetically treated normal saline comprising 60 ppm dissolved oxygen; sometimes referred to as “drug” in this Example).

Patch clamping techniques were utilized to assess the effects of the test material (RNS-60) on epithelial cell membrane polarity and ion channel activity. Specifically, whole cell voltage clamp was performed upon the Bronchial Epithelial line Calu-3 in a bathing solution consisting of: 135 mM NaCl, 5 mM KCl, 1.2 mM CaCl2, 0.8 mM MgCl2, and 10 mM HEPES (pH adjusted to 7.4 with N-methyl D-Glucamine). Basal currents were measured after which RNS-60 was perfused onto the cells.

More specifically, patch pipettes were pulled from borosilicate glass (Garner Glass Co, Claremont, CA) with a two-stage Narishige PB-7 vertical puller and then fire-polished to a resistance between 6-12 Mohms with a Narishige MF-9 microforge (Narishige International USA, East Meadow, N.Y.). The pipettes were filled with an intracellular solution containing (in mM): 135 KCl, 10 NaCl, 5 EGTA, 10 Hepes, pH was adjusted to 7.4 with NMDG (N-Methyl-D-Glucamine).

The cultured Calu-3 cells were placed in a chamber containing the following extracellular solution (in mM): 135 NaCl, 5 KCl, 1.2 CaCl2, 0.5 MgCl2 and 10 Hepes (free acid), pH was adjusted to 7.4 with NMDG.

Cells were viewed using the 40× DIC objective of an Olympus IX71 microscope (Olympus Inc., Tokyo, Japan). After a cell-attached gigaseal was established, a gentle suction was applied to break in, and to attain the whole-cell configuration. Immediately upon breaking in, the cell was voltage clamped at −120, −60, −40 and 0 mV, and was stimulated with voltage steps between ±100 mV (500 ms/step). After collecting the whole-cell currents at the control condition, the same cell was perfused through bath with the test fluid comprising same extracellular solutes and pH as for the above control fluid, and whole-cell currents at different holding potentials were recorded with the same protocols.

Electrophysiological data were acquired with an Axon Patch 200B amplifier, low-pass filtered at 10 kHz, and digitized with 1400A Digidata (Axon Instruments, Union City, Calif.). The pCLAMP 10.0 software (Axon Instruments) was used to acquire and to analyze the data. Current (I)-to-voltage (V) relationships (whole cell conductance) were obtained by plotting the actual current value at approximately 400 msec into the step, versus the holding potential (V). The slope of the IN relationship is the whole cell conductance.

Drugs and Chemicals. Whenever indicated, cells were stimulated with a cAMP stimulatory cocktail containing 8-Br-cAMP (500 mM), IBMX (isobutyl-1-methylxanthie, 200 mM) and forskolin (10 mM). The cAMP analog 8-Br-cAMP (Sigma Chem. Co.) was used from a 25 mM stock in H2O solution. Forskolin (Sigma) and IBMX (Sigma) were used from a DMSO solution containing both 10 mM Forskolin and 200 mM IBMX stock solution. The data obtained are expressed as the mean±SEM whole cell current for 5-9 cells.

### Results:

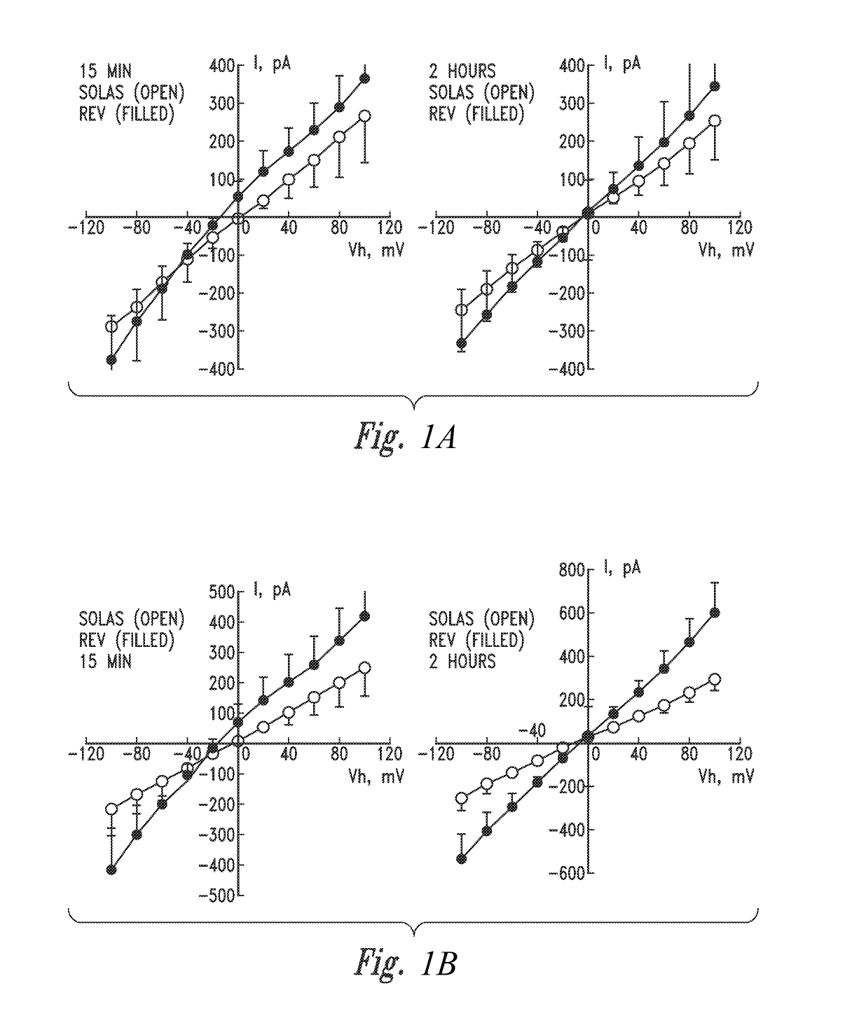

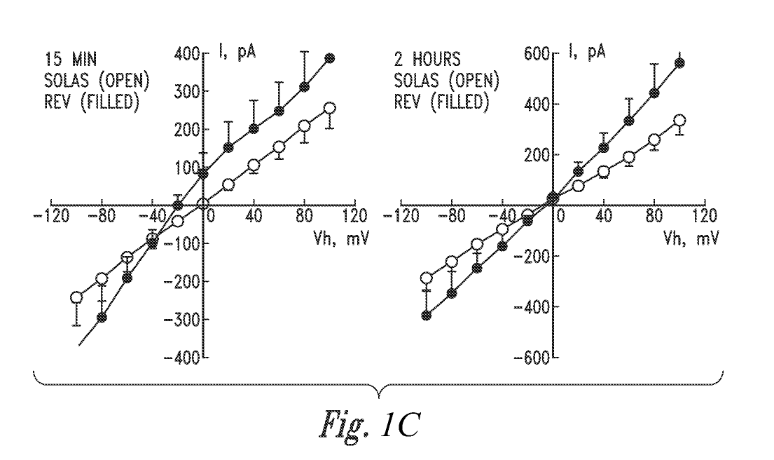

FIGS. 1A-C show the results of a series of patch clamping experiments that assessed the effects of the electrokinetically generated fluid (e.g., RNS-60 and Solas) on epithelial cell membrane polarity and ion channel activity at two time-points (15 min (left panels) and 2 hours (right panels)) and at different voltage protocols (A, stepping from zero mV; B, stepping from −60 mV; and C, stepping from −120 mV). The results indicate that the RNS-60 (filled circles) has a larger effect on whole-cell conductance than Solas (open circles). In the experiment similar results were seen in the three voltage protocols and at both the 15 minute and two-hour incubation time points.

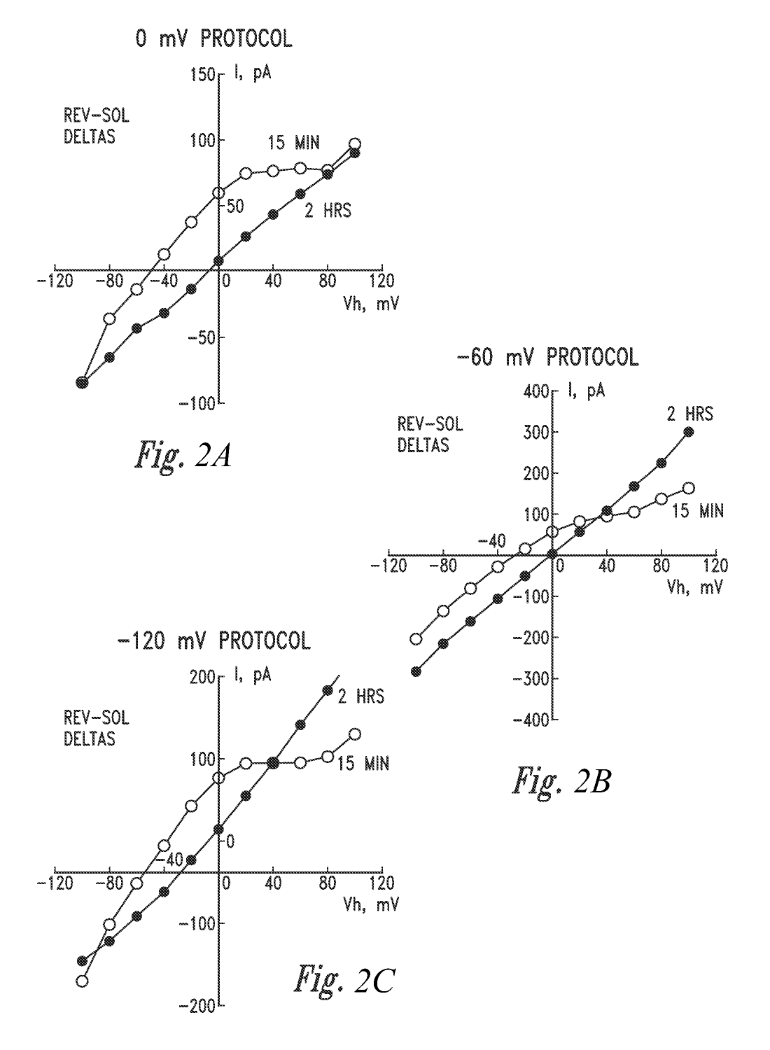

FIGS. 2A-C show graphs resulting from the subtraction of the Solas current data from the RNS-60 current data at three voltage protocols (“Delta currents”) (A, stepping from zero mV; B, stepping from −60 mV; and C, stepping from −120 mV) and the two time-points (15 mins (open circles) and 2 hours (filled circles)). These data indicated that at the 15 minute time-point with RNS-60, there is a non-linear voltage-dependent component that is absent at the 2 hour time point.

As in previous experiments, data with “Normal” saline gave a very consistent and time-independent conductance used as a reference. The present results were obtained by matching groups with either Solas or RNS-60 saline, and indicate that exposure of Calu-3 cells to the RNS-60 saline under basal conditions (without cAMP, or any other stimulation), produces time-dependent effect(s), consistent with the activation of a voltage-regulated conductance at shorter incubation times (15 min). This phenomenon was not as apparent at the two-hour incubation point. As described elsewhere herein, the linear component is more evident when the conductance is increased by stimulation with the cAMP “cocktail”. Nonetheless, the two-hour incubation time showed higher linear conductance for both the RNS-60 and the Solas saline, and in this case, the RNS-60 saline doubled the whole cell conductance as compared to Solas alone. This evidence indicates that at least two contributions to the whole cell conductance are affected by the RNS-60 saline, namely the activation of a non-linear voltage regulated conductance, and a linear conductance, which is more evident at longer incubation times.

### Second Set of Experiments (Effect on Calcium Permeable Channels)

### Methods for Second Set of Experiments:

See above for general patch clamp methods. In the following second set of experiments, yet additional patch clamp studies were performed to further confirm the utility of the inventive electrokinetically generated saline fluids (RNS-60 and Solas) to modulate whole-cell currents, using Calu-3 cells under basal conditions, with protocols stepping from either zero mV or -120 mV holding potentials.

The whole-cell conductance in each case was obtained from the current-to-voltage relationships obtained from cells incubated for 15 min with either saline. To determine whether there is a contribution of calcium permeable channels to the whole cell conductance, and whether this part of the whole cell conductance is affected by incubation with RNS-60 saline, cells were patched in normal saline after the incubation period (entails a high NaCl external solution, while the internal solution contains high KCl). The external saline was then replaced with a solution where NaCl was replaced by CsCl to determine whether there is a change in conductance by replacing the main external cation. Under these conditions, the same cell was then exposed to increasing concentrations of calcium, such that a calcium entry step is made more evident.

### Results:

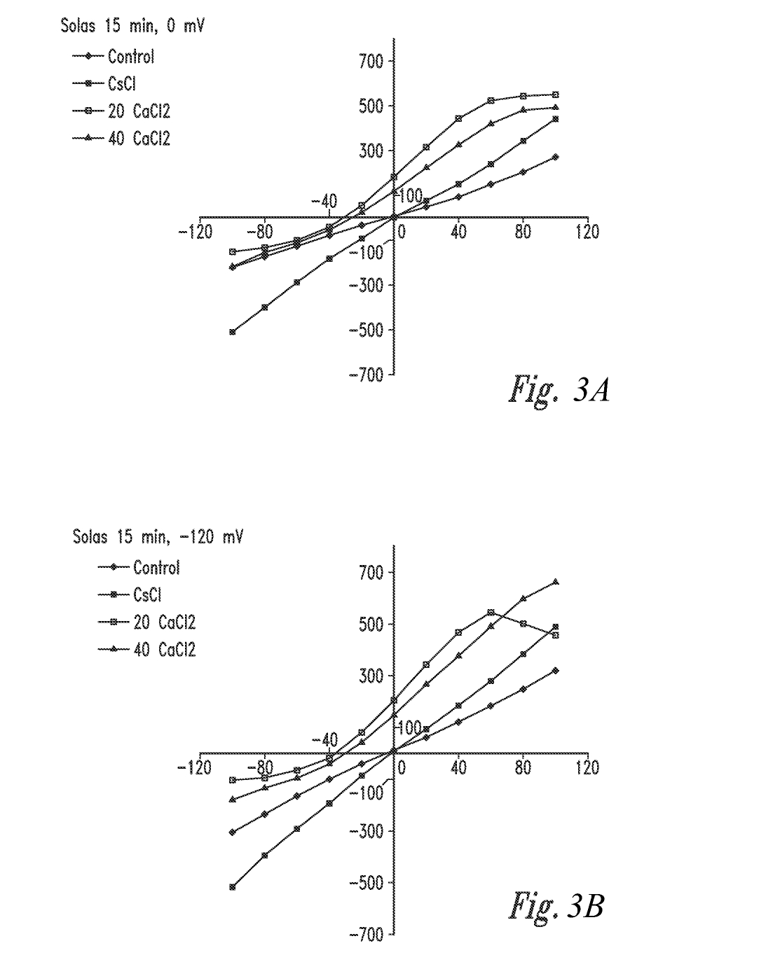

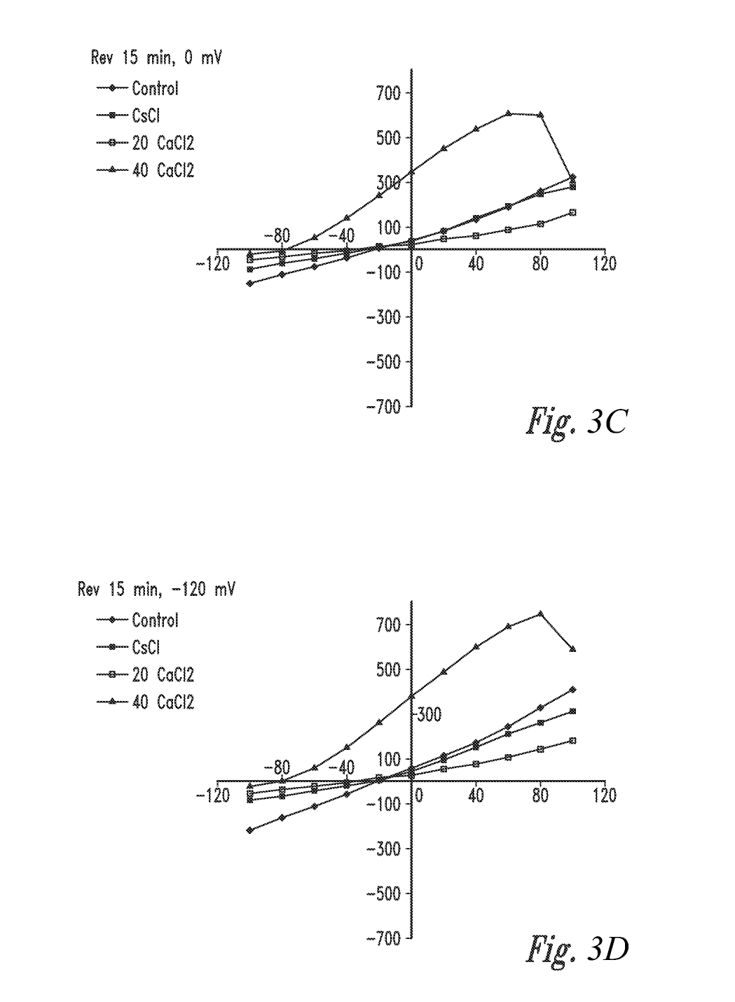

FIGS. 3A-D show the results of a series of patch clamping experiments that assessed the effects of the electrokinetically generated fluid (e.g., Solas (panels A and B) and RNS-60 (panels C and D)) on epithelial cell membrane polarity and ion channel activity using different external salt solutions and at different voltage protocols (panels A and C show stepping from zero mV, whereas panels B and D show stepping from −120 mV). In these experiments one time-point of 15 minutes was used. For Solas (panels A and B) the results indicate that: 1) using CsCl (square symbols) instead of NaCl as the external solution, increased whole cell conductance with a linear behavior when compared to the control (diamond symbols); and 2) CsCl₂ at both 20 mM CsCl₂ (circle symbols) and 40 mM CsCl₂ (triangle symbols) increased whole cell conductance in a non-linear manner. For RNS-60 (panels C and D), the results indicate that: 1) using CsCl (square symbols) instead of NaCl as the external solution had little effect on whole cell conductance when compared to the control (diamond symbols); and 2) CsCl₂ at 40 mM (triangle symbols) increased whole cell conductance in a non-linear manner.

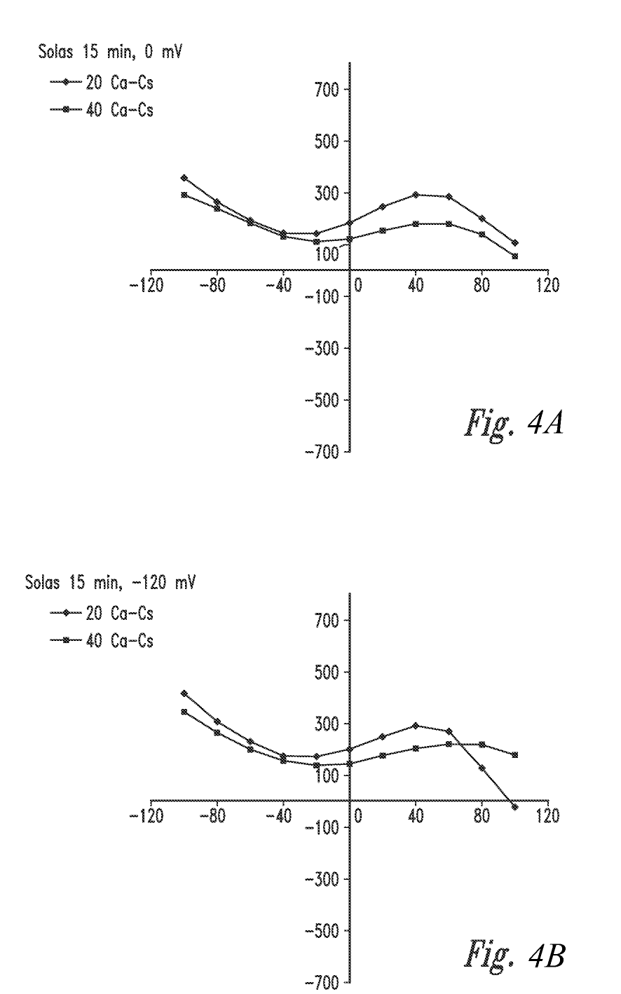

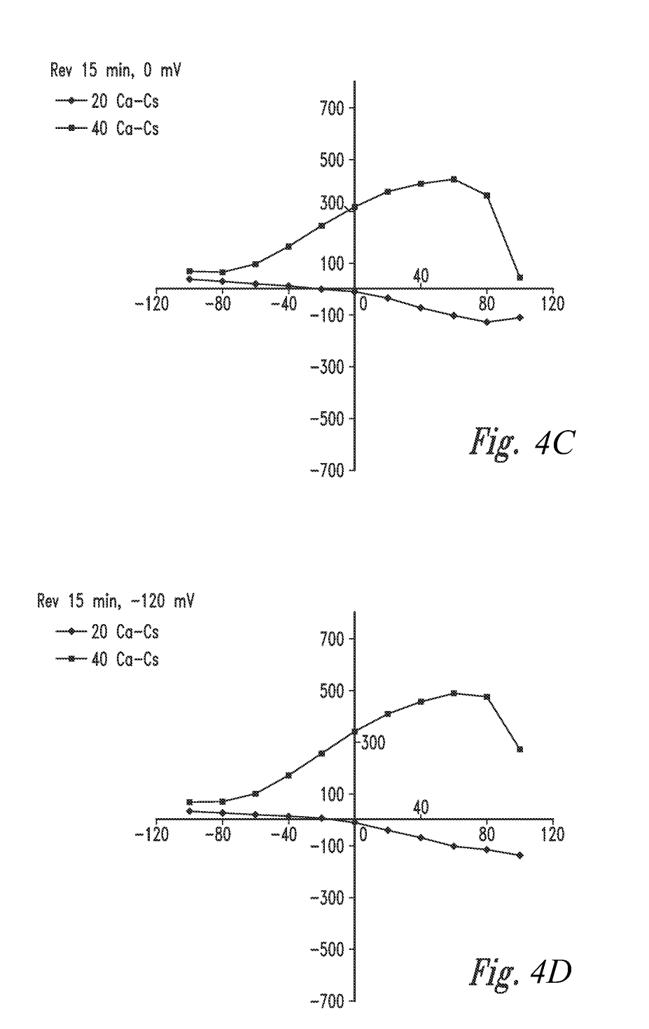

FIGS. 4A-D show the graphs resulting from the subtraction of the CsCl current data (shown in FIG. 3) from the 20 mM CsCl₂ (diamond symbols) and 40 mM CsCl₂ (square symbols) current data at two voltage protocols (panels A and C, stepping from zero mV; and B and D, stepping from −120 mV) for Solas (panels A and B) and RNS-60 (panels C and D). The results indicate that both Solas and RNS-60 solutions activated a calcium-induced non-linear whole cell conductance. The effect was greater with RNS-60 (indicating a dosage responsiveness), and with RNS-60 was only increased at higher calcium concentrations. Moreover, the non-linear calcium dependent conductance at higher calcium concentration was also increased by the voltage protocol.

The data of this second set of experiments further indicates an effect of RNS-60 saline and Solas saline for whole cell conductance data obtained in Calu-3 cells. The data indicate that 15-min incubation with either saline produces a distinct effect on the whole cell conductance, which is most evident with RNS-60, and when external calcium is increased, and further indicates that the RNS-60 saline increases a calcium-dependent non-linear component of the whole cell conductance.

The accumulated evidence suggests activation by Revalesio saline of ion channels, which make different contributions to the basal cell conductance.

Taken together with Applicants' other data (e.g., the data of Applicants other working Examples) particular aspects of the present invention provide compositions and methods for modulating intracellular signal transduction, including modulation of at least one of membrane structure, membrane potential or membrane conductivity, membrane proteins or receptors, ion channels, lipid components, or intracellular components with are exchangeable by the cell (e.g., signaling pathways, such as calcium dependant cellular signaling systems, comprising use of the inventive electrokinetically generated solutions to impart electrochemical and/or conformational changes in membranous structures (e.g., membrane and/or membrane proteins, receptors or other membrane components) including but not limited to GPCRs and/or g-proteins. According to additional aspects, these effects modulate gene expression, and may persist, dependant, for example, on the half lives of the individual messaging components, etc.

### Example 3

### (The Inventive Electrokinetic Fluid was Shown to be Substantially Efficacious in a Dose-Responsive Manner in an Art-Recognized Acute Experimental Allergic (Autoimmune) Encephalomyelitis (EAE) Rat MBP Model of Multiple Sclerosis(MS))

### Overview:

In this working EXAMPLE, the inventive electrokinetic fluid RNS-60 was evaluated at two doses, in both prophylactic and therapeutic administration regimens, in an art-recognized Myelin Basic Protein MBP induced acute Experimental Allergic Encephalomyelitis (EAE) rat model. The inventive electrokinetic fluid RNS-60 was shown to be substantially efficacious in a dose-responsive manner. Both the therapeutic (daily administration of RNS-60 beginning concomitant with MBP injection) and prophylactic (daily administration of RNS-60 beginning seven days prior to MBP injection) RNS-60 dosage regimens showed a marked decrease, as well as a delayed onset (in the high dose groups) of clinical score. According to particular aspects of the present invention, therefore, the inventive electrokinetic compositions have substantial utility for treating, including alleviating and preventing, the symptoms of EAE in an art-recognized rat model of human MS. According to further aspects of the present invention, therefore, the inventive electrokinetic compositions have substantial utility for treating, including alleviating and preventing, the symptoms of MS in afflicted mammals (preferably humans). In yet further aspects, the inventive electrokinetic compositions cross the Blood Brain Barrier (BBB), and thus provided a novel method for treating inflammatory conditions of the central nervous system.

Multiple Sclerosis (MS). Multiple Sclerosis (MS) is a demyelinating disease of the central nervous system (CNS), and is one of the most common disabling neurological diseases in young adults. The main characteristics of this disease are focal areas of demyelination and inflammation. The disease course is unpredictable and life-long, and affects women more commonly than men. The etiology of the disease appears to be dependent on genetic and environmental factors. In the periphery, antigen is bound by antigen presenting cells (APC) via MCH II. Th0 cells bind to the antigen and undergo activation and differentiation. Adhesion molecules and matrix metalloproteases (MMPs) help the Th1 cells to bind and penetrate the Blood Brain Barrier (BBB). Upon crossing the BBB into the CNS, Th1 cells engage antigen-MHC complexes and produce pro-inflammatory cytokines leading to damage in the CNS. The autoimmune system recognizes myelin proteins as foreign and begins to attack. Historically, while Th1 cells are thought to play a predominant role in the pathology of the disease, recent evidence indicates that a proinflammatory cascade of Th17 cells, IL-6 and TGF-β plays a critical role in the pathogenesis of EAE and MS.

Experimental Autoimmune Encephalomyelitis (EAE). Experimental Autoimmune Encephalomyelitis (EAE), also called Experimental Allergic Encephalomyelitis, is a non-human animal model of Multiple Sclerosis (MS). While not MS, the different forms and stages of EAE resemble the various forms and stages of MS very closely in a large number of ways. More specifically, EAE is an acute or chronic-relapsing, acquired, inflammatory and demyelinating autoimmune disease. The animals are injected with the whole or parts of various proteins (e.g., Myelin Basic Protein (MBP), Proteolipid Protein (PLP), and Myelin Oligodendrocyte Glycoprotein (MOG)) that make up myelin, the insulating sheath that surrounds nerve cells (neurons), to induce an autoimmune response against the animal's own myelin that closely resembles MS in humans. EAE has been induced in a number of different animal species including mice, rats, guinea pigs, rabbits, macaques, rhesus monkeys and marmosets. For various reasons including the number of immunological tools, the availability, lifespan and fecundity of the animals and the resemblance of the induced disease to MS, mice and rats are the most commonly used species. The acute rat EAE model has a strong inflammatory component and is therefore an appropriate model in which to investigate the therapeutic potential of an agent that targets immune events in MS.

MBP-induced EAE. MPB in Lewis rats following one dose will lead to relapse that is characterized mainly by hind paw paralysis. Lewis rats are subjected to MBP injection on day 0. Disease develops between day 12-16, with full disease recovery occurring between days 18-21. The model is self limiting and does not show demyelination.

### Materials and Methods:

Production and Characterization of the test fluid (RNS-60). Filter sterilized RNS-60 was prepared by Applicants according to methods described in US2008/0219088 (published on 11 Sep. 2008), US2008/0281001 (published on 11 Nov. 2008) and WO2008/052143 (published on 2 May 2008), all of which are incorporated herein by reference in their entirety and particularly for all aspects relating to the apparatus and/or methods for preparing Applicants' inventive electrokinetic fluids. The dissolved oxygen (DO) content of the RNS-60 used was 59 ppm, as determined by the Winkler Titration assay (Y.C. Wong & C.T. Wong. New Way Chemistry for Hong Kong A-Level Volume 4, Page 248. Or Standard Methods for the Examination of Water and Wastewater—20th Edition ISBN 0-87553-235-7). RNS-60 fluid was labeled with a test item (TI) number, receipt date, storage conditions and expiry date. The storage conditions and handling of the RNS-60 was per Applicants' specification to ensure stability at the Testing Facility during testing. Fluid was kept refrigerated at 2-8° C. when not in use. Vials containing fluid were used as single use containers.

Vehicle control fluid. Vehicle control fluid was Normal Saline for injection (0.9%) from Hospira.

Dexamethasone. Dexamethasone was purchased from Sigma (Cat. No. D1756; Lot No. 096K1805). For administration, Dexamethasone (white powder) was diluted in ethanol to achieve a concentration of 1 mg/ml and then diluted again in distilled water to achieve a dose concentration of 0.1 mg/ml.

EAE Induction Items:

MBP antigenic agent. MBP was Myelin Basic Protein from guinea pig (Des-Gly-77, Des-His-78)-MBP (68-84); Cat. No. H-6875; provided by MD Bioscience). MBP was dissolved in physiological saline at a concentration of 2 mg/ml;

CFA sensitizing agent. Complete Freund's Adjuvant (CFA) was from MD Biosciences Division of Morwell Diagnostics GmbH (Cat. No. IMAD-4). CFA suspension, containing heat killed Mycobacterium Tuberculosis H37 Ra at a concentration of 4 mg/ml, was used as supplied; and

MBP/CFA Emulsion (Antigenic/Sensitizing agents). Prior to the single inoculations carried out on study day 0, one volume of MBP solution was mixed with an equal volume of CFA 4 mg/ml by employing two syringes connected by a Luer fitting to thoroughly mix the emulsive mixture to equal a total dose volume of 100 μl/animal. The dose was delivered as 2×50 μl subcutaneous (SC) bilateral injections into the intraplantar paw regions.

Test animals; Rats. Sixty (60) female Lewis rats (6-7 weeks of age at study initiation) were obtained from Harlan Laboratories Israel, Ltd. Weight variation of animals at the time of treatment initiation should not exceed 20% of the mean weight. The health status of the animals used in this study is examined upon their arrival. Only animals in good health were acclimatized to laboratory conditions and used in the study. Prior to entry in the study, the animals were acclimated for at least 5 days. During acclimation and throughout the study duration, animals were housed within a limited access rodent facility and kept in groups of maximum 5 rats in polypropylene cages fitted with solid bottoms and filled with sterile wood shavings as bedding material. Animals were provided ad libitum with a commercial rodent diet and had free access to drinking water, which was supplied to each cage via polyethylene bottles with stainless steel sipper tubes. A feed lot analysis of the diet batch used in the study was included in the archives with the study data. Water was monitored periodically. Automatically controlled environmental conditions were set to maintain temperature at 20-24° C. with a relative humidity (RH) of 30-70%, a 12:12 hour light:dark cycle and 15-30 air changes/hr in the study room. Temperature and RH were monitored daily. The light cycle was monitored by the control clock. Animals were given a unique animal identification using tail marks. This number also appeared on a cage card, visible on the front of each cage. The cage card also contained the study and group numbers, route of administration, gender, strain and all other relevant details as to treatment group.

TABLE 5
Constitution of Test Groups and Dose Levels, listing the 6 experimental
groups comprising the study:

| | | | | Dose | | |
|---|---|---|---|---|---|---|
| | | | | Level | Volume | |
| Group | Group | Test | | (mg/kg/ | Dosage | |
| Number | Size | Material | Route | admin) | (ml/kg) | Regime |
| 1F | n = 10 | Vehicle | IV | 0 | 2 ml for | 7 days prior to |
| | | Control | | | 350 g | disease |
| | | | | | rat | induction until |
| | | | | | | the end of the |
| | | | | | | study |
| 2F | n = 10 | Dexa- | IP | 1 | 10 | Once daily |
| | | meth- | | | | beginning on |
| | | asone | | | | study day 0 |
| 3F | n = 10 | RNS-60 | IV | | 1 ml for | 7 days prior to |
| | | | | | 350 g | disease |
| | | | | | rat | induction until |
| | | | | | | the end of the |
| | | | | | | study |
| 4F | n = 10 | RNS-60 | IV | | 2 ml for | 7 days prior to |
| | | | | | 350 g | disease |
| | | | | | rat | induction until |
| | | | | | | the end of the |
| | | | | | | study |
| 5F | n = 10 | RNS-60 | IV | | 1 ml for | Once daily |
| | | | | | 350 g | beginning on |
| | | | | | rat | study day 0 |
| 6F | n = 10 | RNS-60 | IV | | 2 ml for | Once daily |
| | | | | | 350 g | beginning on |
| | | | | | rat | study day 0 |

Test procedures and Principles of the Acute EAE Murine Model. Experimental Allergic Encephalomyelitis (EAE) is a central nervous system (CNS) autoimmune demyelinating disease that mimics many of the clinical and pathologic features of Multiple Sclerosis (MS). The acute rat model consists of a sensitization period, induced by the single subcutaneous (SC) injection of Myelin basic protein (MBP) emulsified in Complete Freund's Adjuvant (CFA) on day 0 of the study.

A schematic depiction of EAE induction and treatment regimens is shown in FIG. 6).

EAE Induction:

MBP/CF A. As shown in the schematic description in FIG. 6), all animals were subjected on study day 0 (study commencement) to a single inoculum injection consisting of a homogenate emulsive mixture of MBP and CFA (MBP/CFA encephalitogenic emulsive inoculum (100 μg MBP/200 μg CFA) was injected at a total dose volume of 100 μl/animal and delivered as 2×50 μl subcutaneous (SC) bilateral injections into the intraplantar paw regions).

### Treatment:

Treatment Regimen and Procedure. All compounds were prepared fresh each day by a person different than the one scoring the animals. The person that scored the animals received vials marked only with group numbers and was unaware of the treatment.

Route of Administration: (i) RNS-60 (IV); (ii) Vehicle Controls: (IV); and (iii) Positive Controls: (IP).

Dose Levels and Volume Dosages: (i) RNS-60: Low dose 2 ml for 350 g; High dose 4 ml for 350 g; (ii) Vehicle Controls: 0; and (iii) Positive Control (Dexamethasone): 1 mg/kg.

Supportive Care. Unless determined during the course of the study, once EAE experimental effects were expected and/or observed (approximately 8-12 days post the single encephalitogenic inoculation), or when the animals were showing a decrease is body weight greater than 15% from their previous determination or a decrease greater than 20% of their initial body weight measurement, appropriate supportive care was carried out on a case-by-case basis.

Feeding and Watering. An additional water source consisting of chipped pellets or mealy rodent diet, soaked in drinking water is placed on the cage bottom and in front of the crawling/non-mobile animals.

Dehydration. Animals may be subjected to subcutaneous (SC) supplemental fluid therapy with Dextrose 5% solution at least twice daily and up to 2 ml/animal/day until body weight returns to be within 10% of the initial determination.

Urination. Palpation of the animals' abdomen is carried out in order to assist with voiding and to observe whether the animals can empty their bladder.

Other Special Care. Animals' perianal areas and hind legs were cleaned as needed with a moistened gauze pad.

### Observations and Examinations:

Clinical Signs. Throughout the entire 21-day study, careful clinical examinations were carried out and recorded at least once daily in addition to the EAE clinical scoring and assessment (see below). Observations included changes in skin, fur, eyes, mucous membranes, occurrence of secretions and excretions (e.g. diarrhea) and autonomic activity (e.g., lacrimation, salivation, piloerection, pupil size, unusual respiratory pattern), gait, posture and response to handling, as well as the presence of unusual behavior, tremors, convulsions, sleep and coma.

Body Weights. Body weight loss can be the first sign of disease initiation, while a sudden marked weight gain tends to accompany remission of EAE symptoms. Therefore, determination of individual body weights of animals was made shortly before EAE induction on study day 0 (study commencement) and thereafter on a daily basis throughout the entire 21-day observation period.

EAE Clinical Scoring and Assessments. Initially, all animals were examined for signs of any neurological responses and symptoms prior to EAE induction (study day 0) and thereafter examined on a daily basis throughout the entire 21-day observation period. To avoid experimental bias, EAE reactions are determined in a blinded fashion, as much as possible, by a staff member unaware of the specific treatment applied. EAE reactions were scored and recorded according to a classical, art-recognized conventional 0-5 scale in ascending order of severity as shown below in Table 6:

TABLE 6
EAE reactions were scored and recorded according to a
classical, art-recognized conventional 0-5 scale in
ascending order of severity.

| Grade | Signs/Symptoms |
|---|---|
| 0 | No abnormalities |
| 0.5 | Tail weakness distal half |
| 1 | Tail weakness proximal half |
| 1.5 | Hind paw weakness one paw |
| 2 | Hind paw weakness two paws |
| 2.5 | Fore paw paralysis one paw |
| 3 | Fore paw paralysis two paws |
| 4 | Full paralysis |
| 5 | Death |

Blood Samples. On the day of study termination (day 21), all animals were bled 1 hour post injection. Samples were collected on study days 0 (prophylactic groups only), 7, 14, and 21. Plasma was collected in heparinized vials and kept at −20° C. A volume of 300 μl was stored for the blood count analysis and 100 μl was stored and used for further cytokine analysis via Luminex Technology. Blood counts were analyzed for days 0, 7, 14, and 21.

Tissue Collection. At study termination, the animals were perfused with 4% PFA. Brains and spinal cords were collected and kept in 4% PFA.

Humane Endpoints. Animals found in a moribund condition and/or animals showing severe pain and enduring signs of severe distress were humanely euthanized.

### Statistics/Data Evalution:

Evaluation was primarily based on the relative recorded changes in both neurological symptoms and body weights, expressed as absolute values, percentage (%) change and mean group values obtained in all treated groups vs. those of the Vehicle Control. Analysis of the data by appropriate statistical methods was applied to determine significance of treatment effects.

### Animal Care and Use Statement:

This study was performed following approval of an application form submitted to the appropriate Committee for Ethical Conduct in the Care and Use of Laboratory Animals that the study complied with the rules and regulations set forth.

### Results:

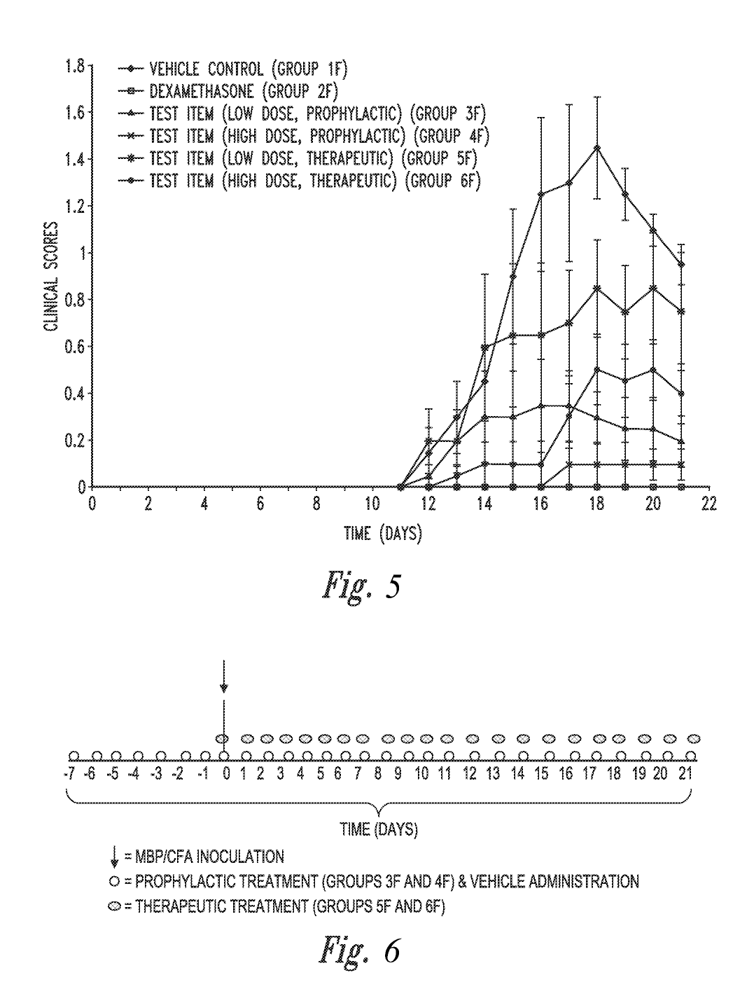

Results of the study are shown in FIG. 5, where time (days after MBP injection) is shown on the X-axis, and “Clinical scores” (see above under “Materials and Methods”) are shown on the Y-axis.

FIG. 5 shows that the inventive electrokinetic fluid (RHS-60) was substantially efficacious in an art-recognized Experimental Autoimmune Encephalomyelitis (EAE) rat model of Multiple Sclerosis (MS) (see above under “Materials and Methods”).

Specifically, compared to the vehicle control group (filled diamonds) over a 17 day period, both the therapeutic (daily administration of RNS-60 beginning concomitant with MBP injection) and prophylactic (daily administration of RNS-60 beginning seven days prior to MBP injection) RNS-60 dosage regimens showed a marked decrease, as well as a delayed onset (in the high dose groups) of clinical score.

The clinical score of the low dose (daily one cc injection) RNS-60 therapeutic group was approximately one-half (½) that of the vehicle control group, while the clinical score of the high dose (daily two cc injection) RNS-60 therapeutic group was not only approximately one-fifth (⅕) to one-tenth ( 1/10) that of the vehicle control group, but also displayed delayed onset.

The clinical score of the low dose (daily one cc injection) RNS-60 prophylactic group was approximately one-third (⅓) that of the vehicle control group, while the clinical score of the high dose (daily two cc injection) RNS-60 prophylactic group was not only zero (no detectable clinical score) through day 16, thereby displaying substantially delayed onset, but when observable at day 17 was less than one-tenth ( 1/10) that of the vehicle control group at the same time point.

According to particular aspects of the present invention, therefore, the inventive electrokinetic compositions have substantial utility for treating, including alleviating and preventing, the symptoms of EAE in art-recognized rat models of human MS.

### Example 4

### (The Inventive Electrokinetic Fluid was Shown to be Effective in Sustaining the Weight of Rats in an Art-Recognized Acute Experimental Allergic (Autoimmune) Encephalomyelitis (EAE) Rat MBP Model of Multiple Sclerosis(MS))

### Overview:

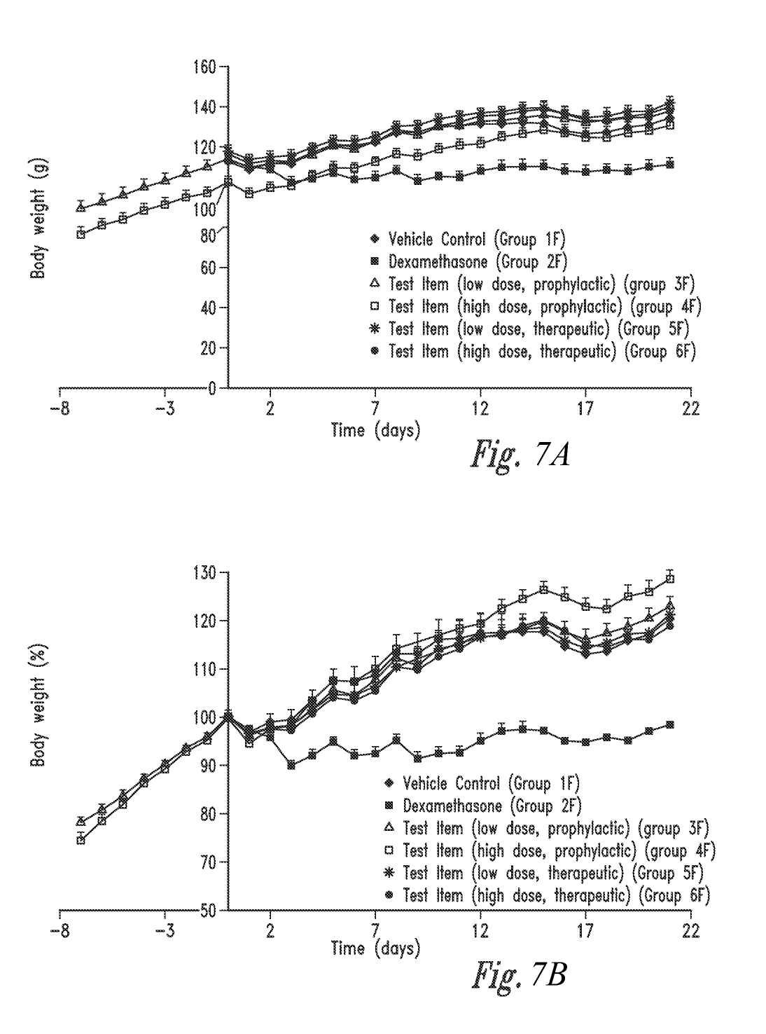

This working EXAMPLE discloses the weight change of rats subjected to the experiment described in Example 7. Body weight loss can be the first sign of disease initiation, while a sudden marked weight gain tends to accompany remission of EAE symptoms. Therefore, determination of individual body weights of animals was made shortly before EAE induction on study day 0 (study commencement) and on a daily basis throughout the 21-day observation period. The effect of the inventive electrokinetic fluid RNS-60 on body weight was shown to be effective in sustaining the weight of rats subjected to the EAE rat model (FIG. 7).

### Body Weight Data:

FIG. 7 shows the body weight in grams (panel A) and as a percentage (panel B) based on 100 grams. After a slight reduction of the mean body weight of in the animals treated in this Example, the mean body weight began to increase until study termination. At study termination, the mean body weight gain was 20% in the Vehicle treated animals (Group 1F). Throughout the study, the Dexamethasone treatment group (Group 2F) which was administered starting on study day 0 had 10% mean body weight loss during the study. At study termination, the Dexamethasone treated animals lost 2% of mean body weight. The prophylactic, low dose treated group (Group 3F) showed up to 4% mean body weight loss on study days 1-3, and then gained 23% of the mean body weight by the day of study termination. The prophylactic, high dose treated group (Group 4F) showed up to 5% mean body weight loss on study days 1-3, and then gained 28% of the mean body weight by the day of study termination. The therapeutic, low dosed treated group (Group 5F) showed up to 4% mean body weight loss on study days 1-3, and then gained 21% of the mean body weight by the day of study termination. The therapeutic, high dose treated group (Group 6F) showed up to 4% mean body weight loss on Study Days 1-3, then gained 19% of the mean body weight by the day of study termination.

Thus the inventive electrokinetic fluid RNS-60 was found to be effective in sustaining the weight of rats subjected to the EAE rat model.

According to particular aspects of the present invention, therefore, the inventive electrokinetic compositions have substantial utility for treating, including alleviating and preventing, the symptoms of EAE in art-recognized rat models of human MS.

### Example 5

(The Inventive Electrokinetic Fluid was Shown to have Little Effect on the Level of White Blood Cells, Neutrophils, and Lymphocytes in Blood Samples Taken from Rat Subjected to the Art-Recognized Acute Experimental Allergic (Autoimmune) Encephalomyelitis (EAE) Rat MBP Model of Multiple Sclerosis(MS))

### Overview:

This working EXAMPLE discloses the level of white blood cells, neutrophils, and lymphocytes in blood samples taken from rats during the experiment as described in Example 7. To determine whether the change in cytokine levels was due to an overall change in white blood cells, Applicants' took blood samples, throughout the experiment, from rats subjected to the EAE experiment.

### Level of White Blood Cells, Neutrophils, and Lymphocytes:

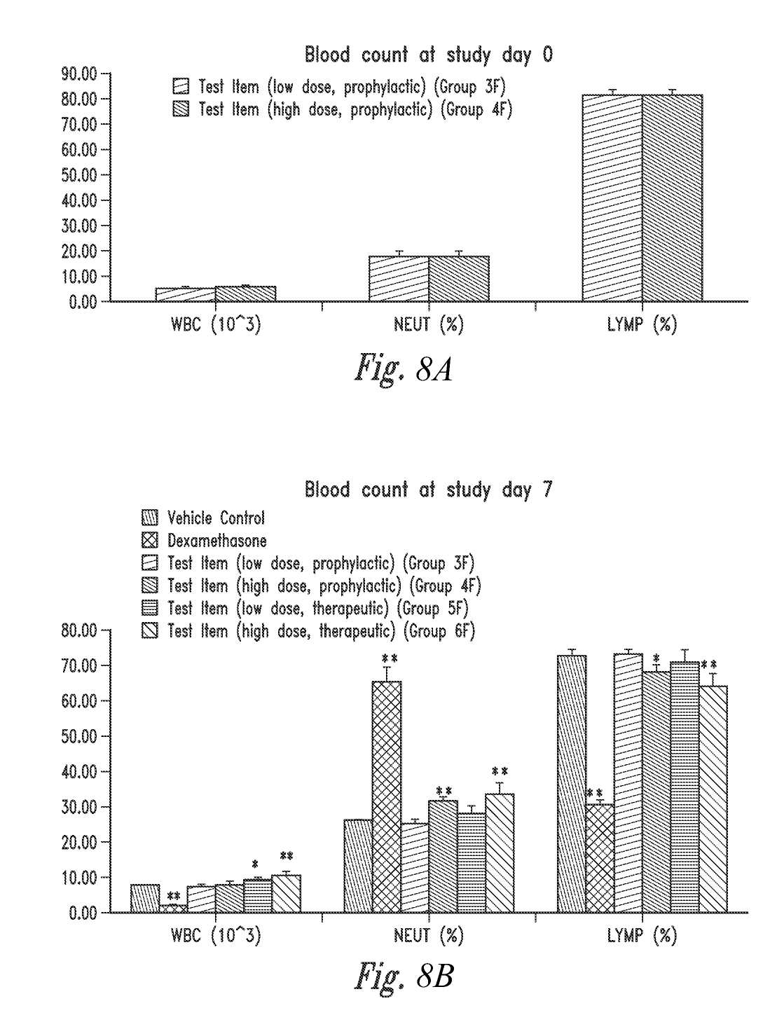

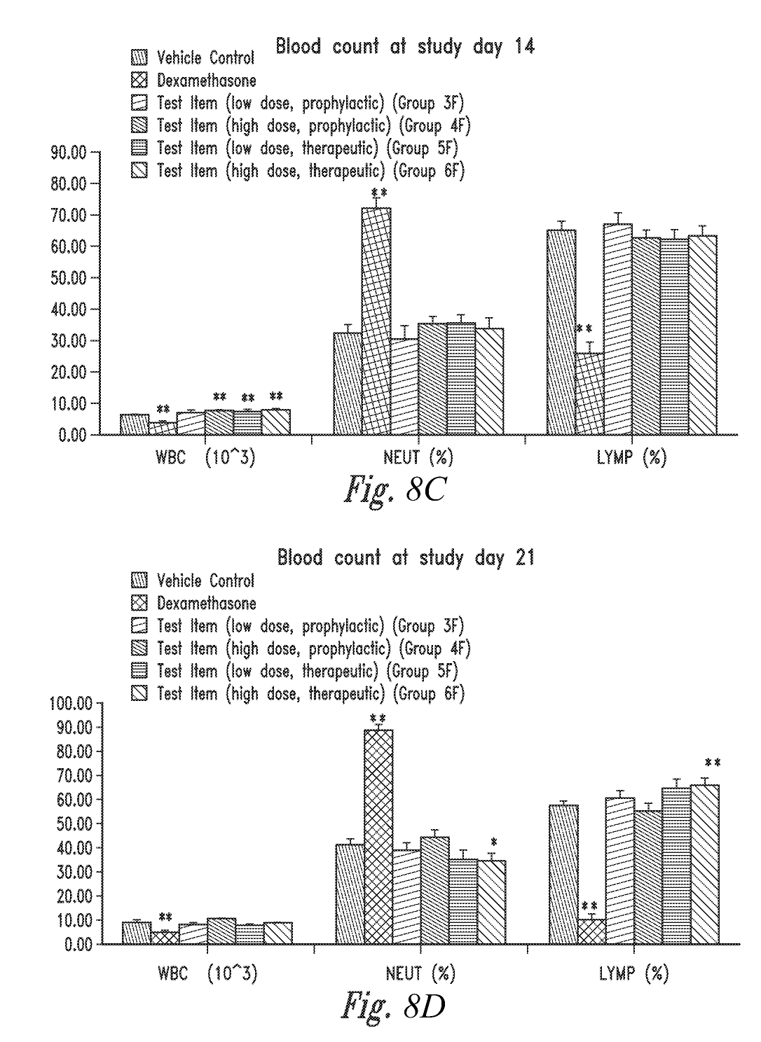

FIGS. 8A-D show the levels of white blood cells, neutrophils, and lymphocytes in blood samples that were collected throughout the EAE experiment.

White blood cells (WBC), neutrophils and lymphocytes were counted one hour after the Test Item was administered on study days 0 (panel A), 7 (panel B), 14 (panel C) and 21 (panel D). The maximum WBC count one hour after the animals were treated with Vehicle on Study Day 7 was 8.23±0.36 points. Treatment with Dexamethasone significantly reduced the average WBC count vs. Vehicle to 2.46±0.38 points (p<0.05). Therapeutic treatment with the Test Item at a low dose (Group 5F) significantly increased the average WBC count vs. Vehicle to 9.59±0.46 points (p<0.1). Therapeutic treatment with the Test Item at a high dose (Group 6F) significantly increased the average WBC count vs. Vehicle to 10.84±0.88 points (p<0.05).

The maximum WBC count one hour after animals were treated with Vehicle on study day 14 was 6.34±0.28 points. Treatment with Dexamethasone significantly reduced the average WBC count vs. Vehicle to 3.79±0.69 points (p<0.05). Prophylactic treatment with the Test Item at the high dose (Group 4F) significantly increased the average WBC count vs. Vehicle to 7.83±0.51 points (p<0.05). Therapeutic treatment with the Test Item at the low dose (Group 5F) significantly increased the average WBC count vs. Vehicle to 7.65±0.52 points (p<0.05). Therapeutic treatment with the Test Item at the high dose (Group 6F) significantly increased the average WBC count vs. Vehicle to 8.05±0.43 points (p<0.05). The maximum WBC count one hour after animals were treated with Vehicle on study day 21 was 9.09±0.75 points. Treatment with Dexamethasone significantly reduced the average WBC count vs. Vehicle to 5.12±0.57 points (p<0.05).

The maximum neutrophils count one hour after animals were treated with the Vehicle on study day 7 was 26.20±1.62 points. Treatment with Dexamethasone significantly increased the average neutrophils count versus vehicle to 65.38±4.62 points (p<0.05). Prophylactic treatment with the Test Item at the high dose (Group 4F) significantly increased the average neutrophils count versus vehicle to 31.90±0.96 points (p<0.05). Therapeutic treatment with the Test Item at the high dose (Group 6F) significantly increased the average neutrophils count versus vehicle to 33.90±2.79 points (p<0.05).

The maximum Neutrophils count one hour after animals were treated with Vehicle on study day 14 was 33.00±2.58 points. Treatment with Dexamethasone significantly increased the average neutrophils count vs. Vehicle to 73.10±3.15 points (p<0.05).

The maximum neutrophils count one hour after animals were treated with Vehicle on study day 21 was 41.40±2.32 points. Treatment with Dexamethasone significantly increased the average neutrophils count vs. Vehicle to 89.33±1.97 points (p<0.05). Therapeutic treatment with the Test Item at the high dose (Group 6F) significantly decreased the average neutrophils count vs. Vehicle to 34.60±3.08 points (p<0.1).

The maximum lymphocytes count one hour after treated with Vehicle on study day 7 was 73.20±1.95 points. Treatment with Dexamethasone significantly reduced the average lymphocytes count vs. Vehicle to 30.63±1.31 points (p<0.05). Prophylactic treatment with the Test Item at the high dose (Group 4F) significantly reduced the mean lymphocytes count vs. Vehicle to 68.30±1.42 points (p<0.1). Therapeutic treatment with the Test Item at the high dose (Group 6F) significantly reduced the average lymphocytes count vs. Vehicle to 64.80.±3.00 points (p<0.05).

The maximum lymphocytes count one hour after treated with Vehicle on study day 14 was 66.10±2.53 points. Treatment with Dexamethasone significantly reduced the average lymphocytes count vs. Vehicle to 26.80±3.23 points (p<0.05).

The maximum lymphocytes count one hour after treated with Vehicle on study day 21 was 57.50±2.09 points. Treatment with Dexamethasone significantly reduced the average lymphocytes count vs. Vehicle to 10.11±2.08 points (p<0.05). Therapeutic treatment with the

Test Item at the high dose (Group 6F) significantly increased the average lymphocytes count vs. Vehicle to 66.20±2.74 points (p<0.05).

Thus the inventive electrokinetic fluid RNS-60 administered prophylactically and therapeutically at the high dose significantly increased the neutrophils count and significantly decreased the lymphocytes count versus the Vehicle at study day 7. The inventive electrokinetic fluid RNS-60 administered prophylactically at the high dose, and therapeutically at both doses, significantly increased the WBC count versus the Vehicle at study day 14. The Test Item RNS60 administered therapeutically at the high dose, significantly decreased the neutrophils count and increased the Lymphocytes count versus the Vehicle at study day 21. Thus the inventive electrokinetic fluid RNS-60 was found to have little effect on the overall levels of WBC, neutrophils, and lymphocytes.

### Example 6

(The Inventive Electrokinetic Fluid was Shown to Effect the Level of Certain Cytokines in Blood Samples Taken from Rat Subjected to the Art-Recognized Acute Experimental Allergic (Autoimmune) Encephalomyelitis (EAE) Rat MBP Model of Multiple Sclerosis(MS))

### Overview:

This working EXAMPLE discloses the level of cytokines as discovered in blood samples taken from rats during the experiment as described in Example 7. The inventive electrokinetic fluid RNS-60 was evaluated in the therapeutic administration regimens, as described in Example 7. The inventive electrokinetic fluid RNS-60 was shown to affect the level of certain cytokines in blood samples taken from rat subjected to the EAE rat model.

Certain cytokines have been shown to have a role in Multiple Sclerosis. In particular interleukin 17 (IL-17), also known as CTLA-8 or IL-17A, has been demonstrated to have elevated levels in the central nervous system in acute and chronic EAE (Hofstetter, H. H., et al., Cellular Immunology (2005), 237:123-130). IL-17 is a pro-inflammatory cytokine which stimulates the secretion of a wide range of other cytokines from various non-immune cells. IL-17 is capable of inducing the secretion of IL-6, IL-8, PGE2, MCP-1 and G-CSF by adherent cells like fibroblasts, keratinocytes, epithelial and endothelial cells and is also able to induce ICAM-1 surface expression, proliferation of T cells, and growth and differentiation of CD34+human progenitors into neutrophils when cocultured in presence of irradiated fibroblasts (Fossiez et al., 1998, Int.Rev.Immunol. 16, 541-551). IL-17 is predominantly produced by activated memory T cells and acts by binding to a ubiquitously distributed cell surface receptor (IL-17R) (Yao et al., 1997, Cytokine, 9, 794-800). A number of homologues of IL-17 have been identified which have both similar and distinct roles in regulating inflammatory responses. For a review of IL-17 cytokine/receptor families see Dumont, 2003, Expert Opin. Ther. Patents, 13, 287-303.

IL-17 may contribute to a number of diseases mediated by abnormal immune responses, such as rheumatoid arthritis and air-way inflammation, as well as organ transplant rejection and antitumour immunity. Inhibitors of IL-17 activity are well known in the art, for example an IL-17R:Fc fusion protein was used to demonstrate the role of IL-17 in collagen-induced arthritis (Lubberts et al., J. Immunol. 2001,167, 1004-1013) and neutralising polyclonal antibodies have been used to reduce peritoneal adhesion formation (Chung et al., 2002, J. Exp. Med., 195, 1471-1478). Neutralising monoclonal antibodies are commercially available (R&D Systems UK).

Thus based on the role IL-17 plays in the pathogenesis of MS, Applicants' examined the effect that inventive electrokinetic fluid RNS-60 had on levels of IL-17 in blood samples taken from rats in the EAE study.

### Cytokine Data:

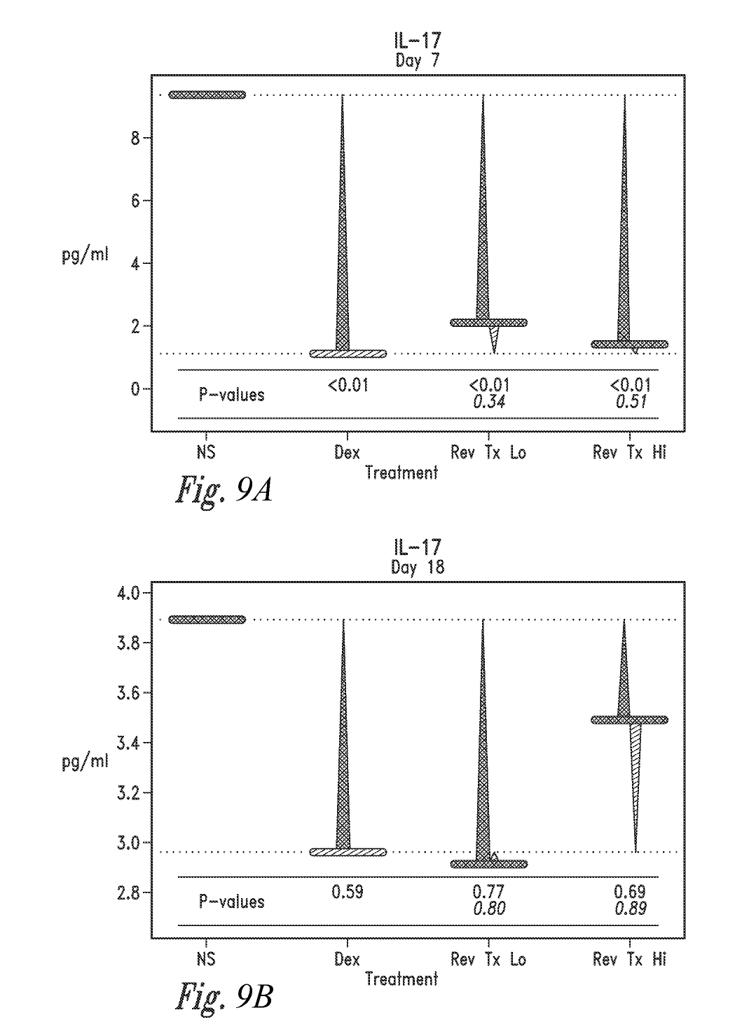

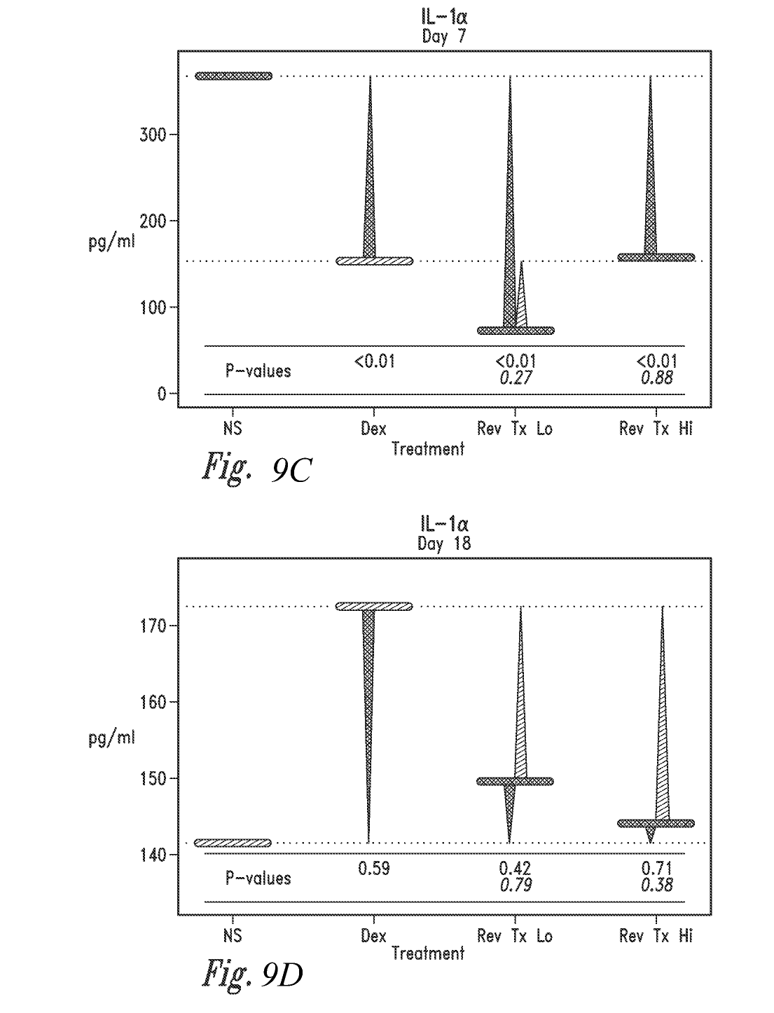

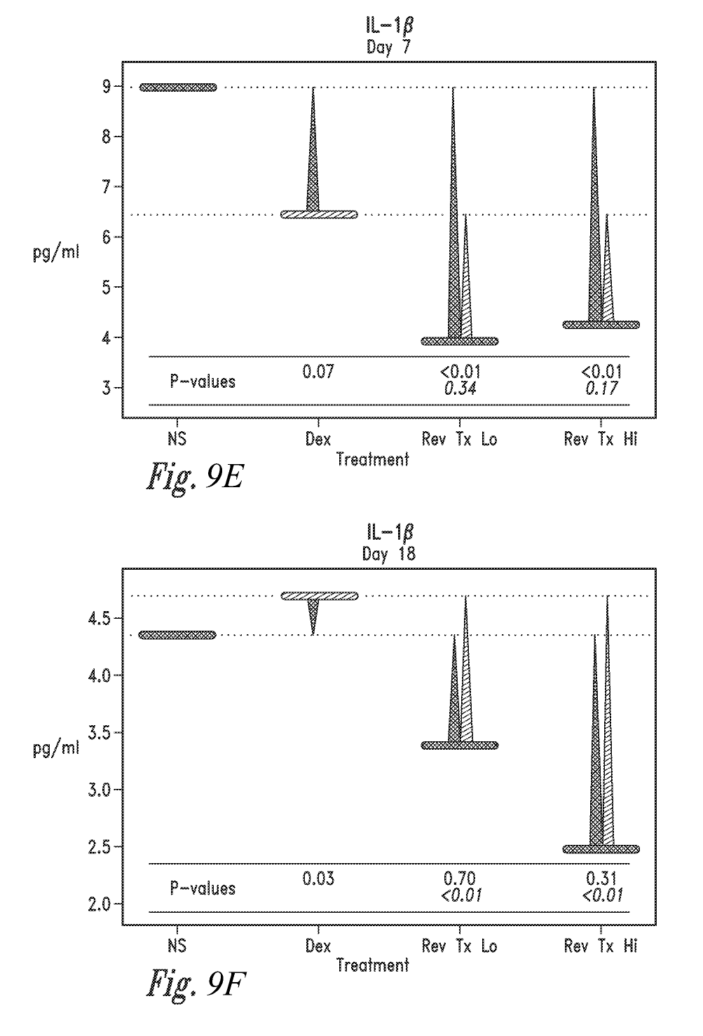

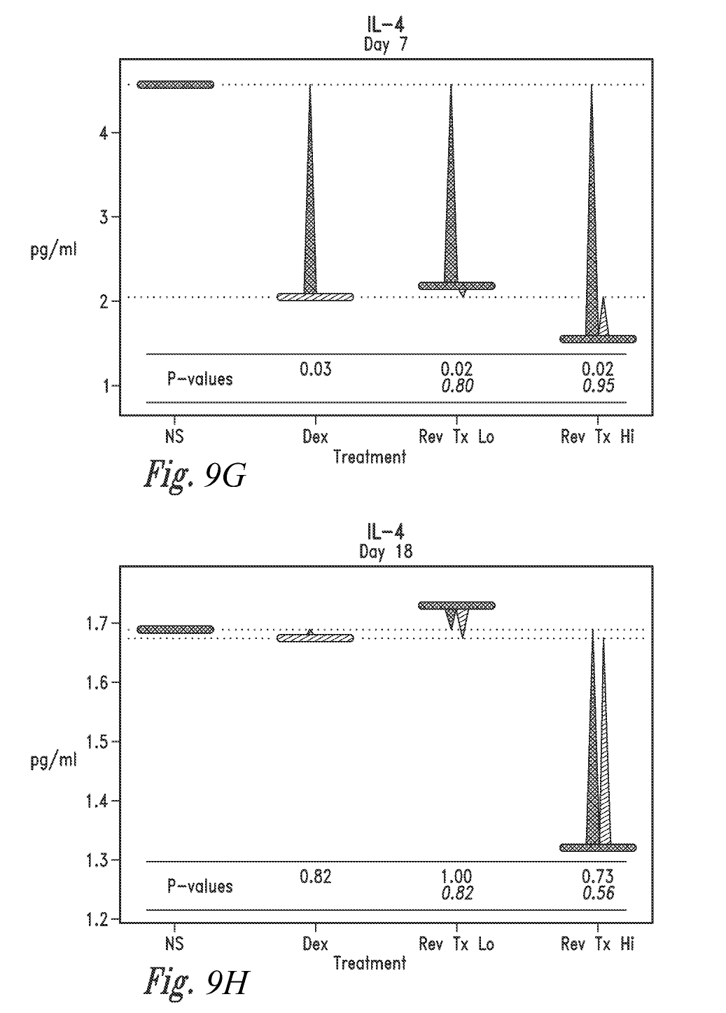

Levels of various cytokines in the blood were analyzed during the study. In brief, all animals were bled 1-hour post injection and plasma was collected in heparinized vials. 100 μl samples were analyzed for various inflammatory cytokines by Luminex technology (using Procarta rat cytokine assay kit PC4127 from Panomics) which enables measurement of multiple cytokines from the same sample, simultaneously. Due the non-Gaussian distributed data and occasional results below the assay detection threshold, the nonparametric Cox regression model for censored data was adapted to compare the different fluids. As show in FIGS. 9A-H, levels of IL1a, IL1b, and IL17 were most notably reduced by both therapeutic treatment doses (high and low) of RNS60. Clinical manifestation of MBP induced EAE starts around day 10 and peaks around day 18. Hence, we considered the day 7 (just prior to disease manifestation) and day 18 (around the peak of the disease) to be the most important time points for cytokine analysis. Systemic levels of IL1a, IL1b and IL17 on days 7 and 18, from 10 animals/group are presented in FIGS. 9A-H.

IL-1 is one of the major pro-inflammatory cytokines and is an upstream mediator of the innate immune responses. IL-1 induces the production of various growth and trophic factors, inflammatory mediators, adhesion molecules and other cytokines directly and indirectly, as well as using a positive feedback loop (A. Basu et al., The type 1 interleukin-1 receptor is essential for the efficient activation of microglia and the induction of multiple proinflammatory mediators in response to brain injury, J. Neurosci. 22 (2002), pp. 6071-6082; P. N. Moynagh, The interleukin-1 signaling pathway in astrocytes: a key contributor to inflammation in the brain, J. Anat. 207 (2005), pp. 265-269). These include important modulators such as NGF, ICAM 1, IL6, TNFα, CSF etc. The progression of MS involves the activation of auto-antigen-reactive T cells in the periphery, followed by invasion into the CNS. IL-1 is crucial in the development of MS as they participate not only in myelin-specific T cell activation but also represent the main mediator of macrophage activation in the periphery [R. Furlan et al., HSV-1-mediated IL-1 receptor antagonist gene therapy ameliorates MOG(35-55)-induced experimental autoimmune encephalomyelitis in C57BL/6 mice, Gene Ther. 14 (2007), pp. 93-98)). In EAE models for MS, both IL-1α and IL-1β have been shown to be mediators of the inflammatory process. Peripheral levels of IL-1β correlate with the clinical course and IL-1β reactivity has been shown during EAE in CNS-infiltrating macrophages and in resident microglial cells ((C. A. Jacobs et al., Experimental autoimmune encephalomyelitis is exacerbated by IL-1 alpha and suppressed by soluble IL-1 receptor, J. Immunol. 146 (1991), pp. 2983-2989)). Therefore, IL-1 is a suitable therapeutic target in EAE and MS. A non-selective inhibitory mechanism of IL-1 has been shown in existing therapeutic agents for MS; that is interferon beta, anti-inflammatory glucocorticoids, immunosuppressants, atorvastatin and omega-3 polyunsaturated fatty acids [F. L. Sciacca et al., Induction of IL-1 receptor antagonist by interferon beta: implication for the treatment of multiple sclerosis, J. Neurovirol. 6 (Suppl. 2) (2000), pp. S33-S37.; R. Pannu et al., Attenuation of acute inflammatory response by atorvastatin after spinal cord injury in rats, J. Neurosci. Res. 79 (2005), pp. 340-350; A. P. Simopoulos, Omega-3 fatty acids in inflammation and autoimmune diseases, J. Am. Coll. Nutr. 21 (2002), pp. 495-505)). As demonstrated in FIG. 9C-F, IV administration of RNS60 effectively lowers the systemic levels of both IL1α and IL1β. For IL1α, RNS60treatment lowered the blood level significantly compared to the vehicle treated group, and was as effective as dexamethasone at this time point. However at the 18 day time point, the treatment has no significant effect on the IL1α systemic level. Systemic levels of IL1β were also reduced significantly after 7 days of IV treatment of RNS60, to the levels comparable to the dexamethsone treatment groups, without any sign of toxic side effects. Although the same trend was noted at the 18 day time point, the differences were not statistically significant when compared to the control group.IL-17 is a also crucial effector cytokine with potent proinflammatory effects. It induces the expression of other proinflammatory cytokines such as tumor necrosis factor-α and chemokines, attracts neutrophilic leukocytes, and enhances the maturation of dendritic cells (Kolls J K, Lindén A.Interleukin-17 family members and inflammation.Immunity. 2004 October; 21(4):467-76). IL-17-producing cells are thought to be essential inflammatory mediators in autoimmune diseases such as collagen-induced arthritis, colitis, psoriasis, and EAE. T helperl7 cells in EAE are CD4+cells and they are present both in the immune periphery and in the inflamed central nervous system in EAE. Moreover, neutralization of IL-17 ameliorates clinical disease, a finding that is paralleled by reduced EAE severity in IL-17-deficient animals ((from Gold and L\ddot{h}der, Interleukin-17—Extended Features of a Key Player in Multiple Sclerosis Am J Pathol. 2008 January; 172(1): 8-10.). 7 day IV treatment with RNS60 caused a significant reduction in IL17 levels in blood, once again to a level similar to dexamethasone treated animals. The same was followed even after 18 days of treatment although the results were not statistically significant. It is important to note that RNS60 is effective not only in lowering the IL1 levels but the combination of the two key cytokines in EAE, IL1 and IL17 with no notable toxic side effects even after 21 days of IV injections.

In addition to IL1 and IL17, a number of other molecules that play critical role in inflammation of the nervous system are also modulated by RIS60. These include Rantes, KC, NGF and ICAM (data not shown).

Thus the inventive electrokinetic fluid RNS-60 had a significant effect on levels of IL-17 in blood samples taken from rats in the EAE study. In addition, since IL-17 stimulates the secretion of IL-6, IL-8, PGE2, MCP-1 and G-CSF, it seems likely that the inventive electrokinetic fluid RNS-60 would have a significant effect on the level of these cytokines in blood. According to particular aspects of the present invention, therefore, the inventive electrokinetic compositions have substantial utility for treating, including alleviating and preventing, the symptoms of EAE in art-recognized rat models of human MS.

### Example 7

(The Inventive Electrokinetic Fluid (e.g., RNS60) was Shown to Inhibit the Expression of both iNOS and IL-1β in a Dose-Dependent Manner in Microglial Cells)

### Overview:

According to particular aspects as described herein, the inventive electrokinetic fluids have substantial utility for treating Parkinson's disease (PD).

Parkinson's disease (PD) is one of the most devastating neurodegenerative disorders in humans. PD may appear at any age, but it is uncommon in people younger than 30. Clinically, PD is characterized by tremor, bradykinesia, rigidity and postural instability. Pathologically, it is indicated by gliosis and progressive degeneration of the dopaminergic neurons associated with the presence of intracytoplasmic inclusions (Lewy bodies) in the substantia nigra pars compacta (SNpc). In postmortem PD brain, dying neurons have been reported to display morphological characteristics of apoptosis, including cell shrinkage, chromatin condensation, and DNA fragmentation. Therefore, development of effective neuroprotective therapeutic approacheso halt the disease progression is of paramount importance. The MPTP mouse model has substantial utility for testing and validating therapeutic approaches against PD.

Microglial activation plays an important role in the pathogenesis of Parkinson's disease (PD) as well as other neurodegenerative disorders. Particular features of PD are modeled in 1-methyl-4-phenyl-1,2,3,6-tetrahydropyridine (MPTP)-intoxicated animals. The neurotoxic effect of MPTP depends on its conversion into MPP⁺. In glial cells, monoamine oxidase B (MAO-B) converts MPTP to MPP+, which then activates glial cells, and recently, it has been shown that MPP⁺ induces the expression of proinflammatory molecules in microglia. In addition, MPP⁺ causes apoptosis of dopaminergic neurons.

In this working EXAMPLE, the ability of RNS60 to modulate the expression of proinflammatory molecules in MPP⁺-stimulatedmicroglial cells was confirmed.

### Materials and Methods:

Briefly, mouse BV-2 microglial cells were incubated with different concentrations of RNS60 and normal saline (NS) for 1 hour followed by stimulation with 2 μM MPP⁺ under serum-free conditions. After 6 hours, total RNA was isolated and mRNA of iNOS and IL-1β was measured by semi-quantitative RT-PCR. Data are representative of three independent experiments.

### Results:

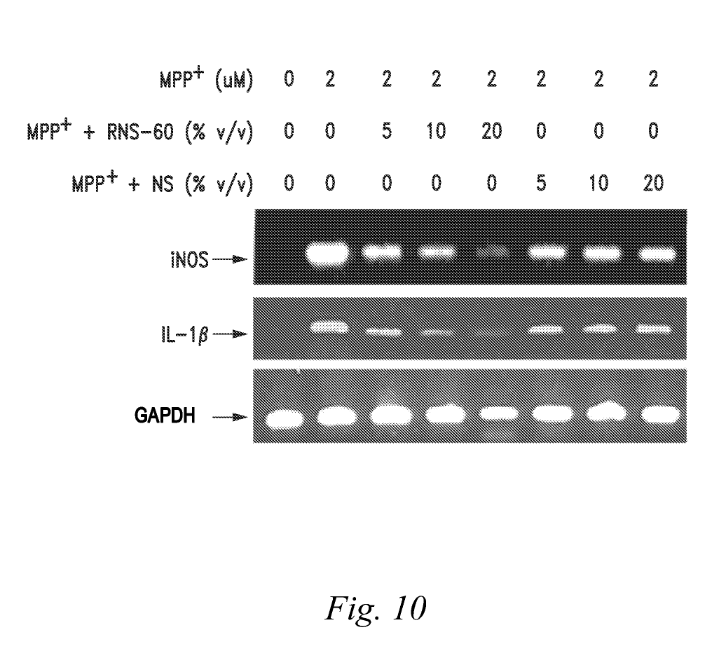

As evidenced by semi-quantitative RT-PCR analysis in FIG. 10, MPP⁺ alone induced the expression of inducible nitric oxide synthase (iNOS) and interleukin-1β(IL-1β) mRNAs in mouse BV-2 microglial cells. Signficantly, RNS60 inhibited the expression of both iNOS and IL-1β in a dose-dependent manner in microglial cells (FIG. 10). By contrast, under similar experimental condition, the normal saline control (NS) had no effect on the expression of these two proinflammatory genes (FIG. 10) indicating the specificity of the effect.

Specifically, FIG. 10 shows that the inventive electrokinetic fluid (RNS-60), but not control normal saline (NS), attenuates MPP⁺-induced expression of inducible nitric oxide synthase (iNOS) and interleukin-1β(IL-1β) in mouse microglial cells. BV-2 microglial cells preincubated with different concentrations of RNS60 and normal saline (NS) in serum-free media for 1 h were stimlated with MPP+(a Parkinsonian toxin). After 6 h of stimulation, total RNA was isolated and the mRNA expression of iNOS and IL-1β was analyzed by semi-quantitative RT-PCR. Results represent three independent experiments.

According to particular aspects therefore, because MPP⁺ is a Parkinsonian toxin, these results indicate that RNS60 has a protective effect in an art-recognized MPTP-induced mouse model of Parkinson's disease.

According to particular aspects, the inventive electrokinetic fluids have substantial utility for treating Parkinson's disease (PD).

### Example 8

(The Inventive Electrokinetic Fluid (e.g., RNS60) was Shown to Protect Nerve Cells and Primary Human Neurons from Amyloid-β Toxicity)

### Overview:

According to particular aspects as described herein, the inventive electrokinetic fluids have substantial utility for treating Alzheimer's disease (AD).

Alzheimer's disease (AD) is a neurodegenerative disorder resulting in progressive neuronal death and memory loss. Increased TUNEL staining in postmortem AD brains indicates that neurons in the brains of AD patients die through apoptosis. Fibrillar amyloid-β peptides participate in the pathophysiology of AD. Neuropathologically, the disease is characterized by neurofibrillary tangles and neuritic plaques composed of aggregates of β-amyloid (Aβ) protein, a 40-43 amino acid proteolytic fragment derived from the amyloid precursor protein, and phosphorylated tau. It has been found that over-expression of the Aβ peptides intracellularly in transgenic mice causes chromatin segmentation, condensation, and increased TUNEL staining Cell culture studies have also shown that Aβ peptides are apoptotic and cytotoxic to neuronal cells, and It has been shown that fibrillar Aβ1-42 peptides are capable of inducing apoptosis in neuronal cells.

Additionally, studies are increasingly being directed at characterizing the link between inflammation and AD, and widespread glial activation has been found around plaques and tangles.

In this EXAMPLE, the effect of RNS60 in blocking Aβ(1-42)-induced apoptosis in human SHSY5Y nerve cells and primary human neurons was confirmed.

### Materials and Methods:

Fragmented DNA of SHS5Y human neuronal cells was detected in situ by the terminal deoxynucleotidyltransferase (TdT)-mediated binding of 3′-OH ends of DNA fragments generated in response to fibrillar Aβ1-42, using a commercially available kit (TdT FragEL™) from Calbiochem. Briefly, cover slips were treated with 20 μg/ml proteinase K for 15 min at room temperature and washed prior to TdT staining Neurons were isolated as described previously and cultured (1,2).

### Results:

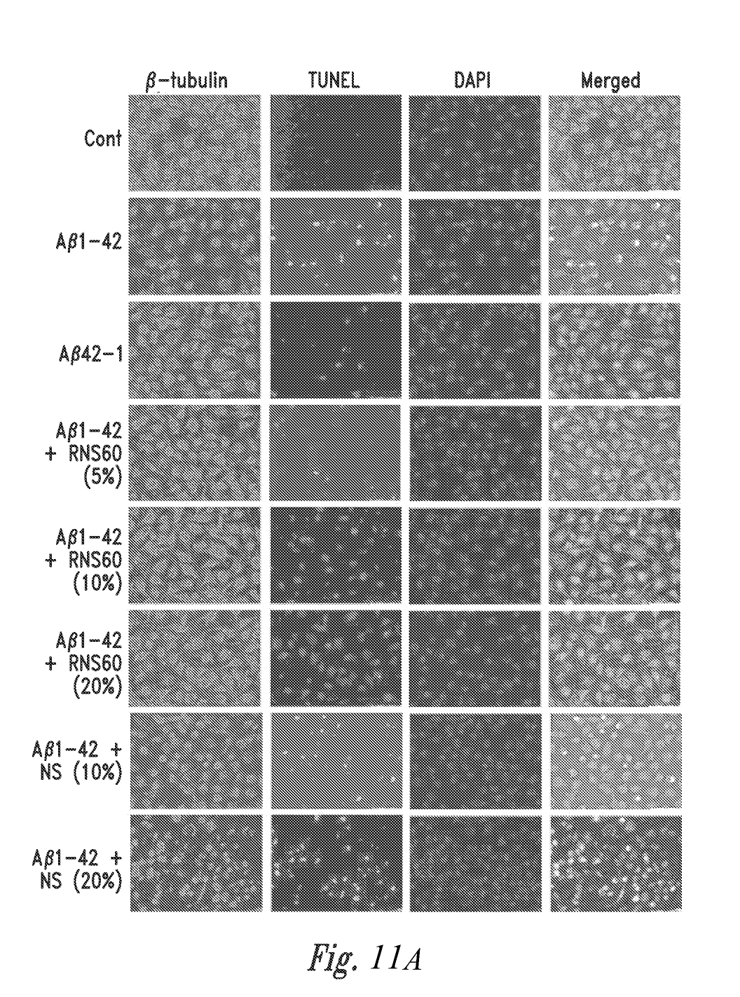

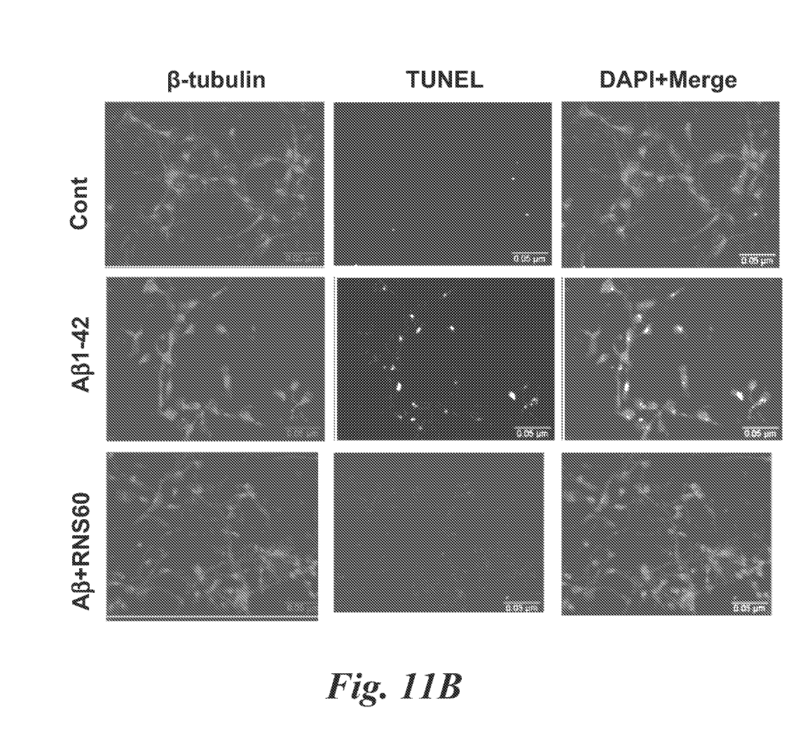

As demonstrated in FIGS. 11A and B, fibrillar Aβ1-42 peptides markedly induced the formation of apoptotic bodies in neuronal cells. We also observed loss of neuronal processing after Aβ1-42 treatment (2^{nd} row; FIG. 11A). In contrast, reverse peptides Aβ2-1 were unable to induce neuronal apoptosis and loss ofprocesses (3^{rd} row; FIG. 11A). Significantly, RNS60 at different doses tested markedly blocked Aβ(1-42)-induced apoptosis and preserved processes in neuronal cells (4^{th}, 5^{th} & 6^{th} rows; FIGS. 11A and B). By contrast, normal saline control fluid (NS) had no effect on Aβ(1-42)-induced apoptosis and loss of processes (7^{th} & 8^{th} rows; FIG. 11A).

Specifically, FIG. 11A shows that RNS60, but not normal saline control (NS), suppresses fibrillar Aβ(1-42)-mediated apoptosis of human SHSY5Y neuronal cells. After differentiation, SHSY5Y cells were incubated with different concentrations of either RNS60 or NS for 1 h followed by insult with 1 μM fibrillar Aβ(1-42) peptides. After 18 h of treatment, apoptosis was monitored by TUNEL (Calbiochem). Aβ(42-1) peptides were also incubated as control. Results represent three independent experiments.

In addition, FIG. 11B, 2^{nd} and 3^{rd} row shows that RNS60 suppresses fibrillar Aβ(1-42)-mediated apoptosis of primary human neurons. Neurons were incubated with RNS60 for 1 h followed by insult with 1 μM fibrillar Aβ(1-42) peptides. After 18 h of treatment, apoptosis was monitored by TUNEL (Calbiochem). Aβ(42-1) peptides were also incubated as control. Results represent three independent experiments.

These results indicate that the etiological reagent of AD (fibrillar Aβ1-42) induces apoptosis in neurons via an RNS60-sensitive pathway and that RNS60 can strongly inhibit fibrillar induced apoptosis in both cultured and primary neurons.

According to particular aspects, the inventive electrokinetic fluids have substantial utility for treating Alzheimer's disease (AD), and in preferred aspects, preventing or slowing progression of AD.

### Example 9

### (The Inventive Electrokinetic Fluid was Shown to be Substantially Efficacious in Suppressing Clinical Score in a Dose-Responsive Manner in an Art-Recognized Mouse MOG Model of Multiple Sclerosis (MS))

### Overview:

In this working EXAMPLE, the inventive electrokinetic fluid RNS-60 was evaluated at two doses, in therapeutic administration regimens, in an art-recognized experimental allergic encephalomyelitis (EAE) mouse MOG model of Multiple Sclerosis (MS).

### Materials and Methods:

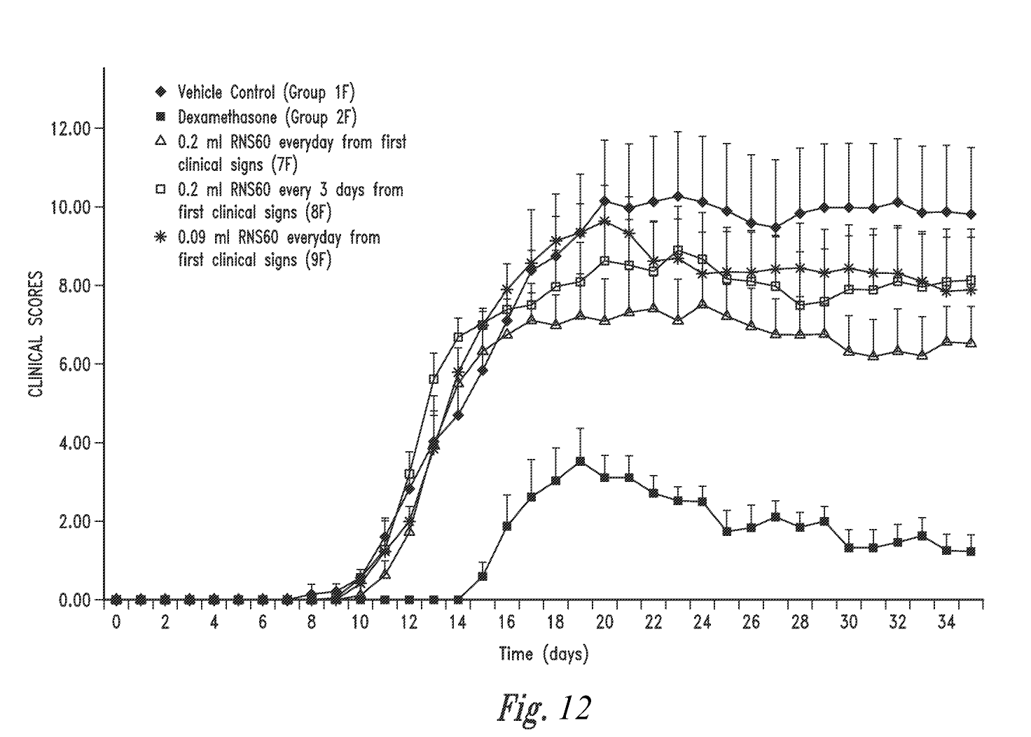

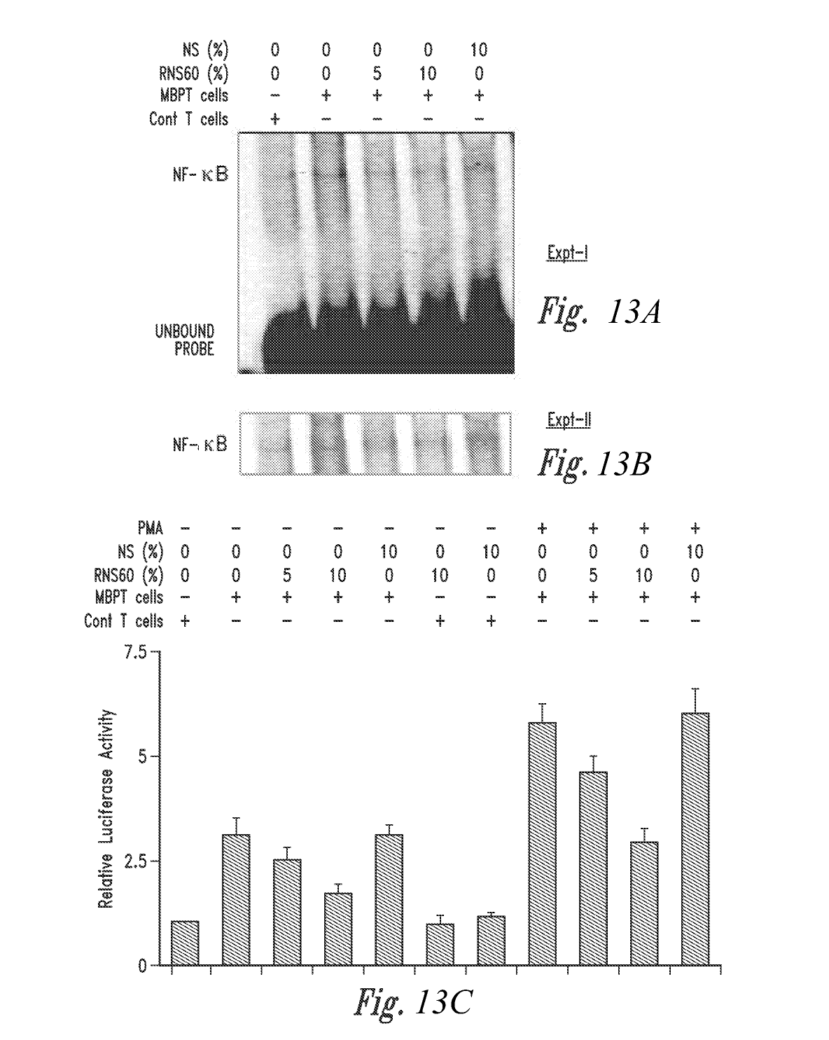

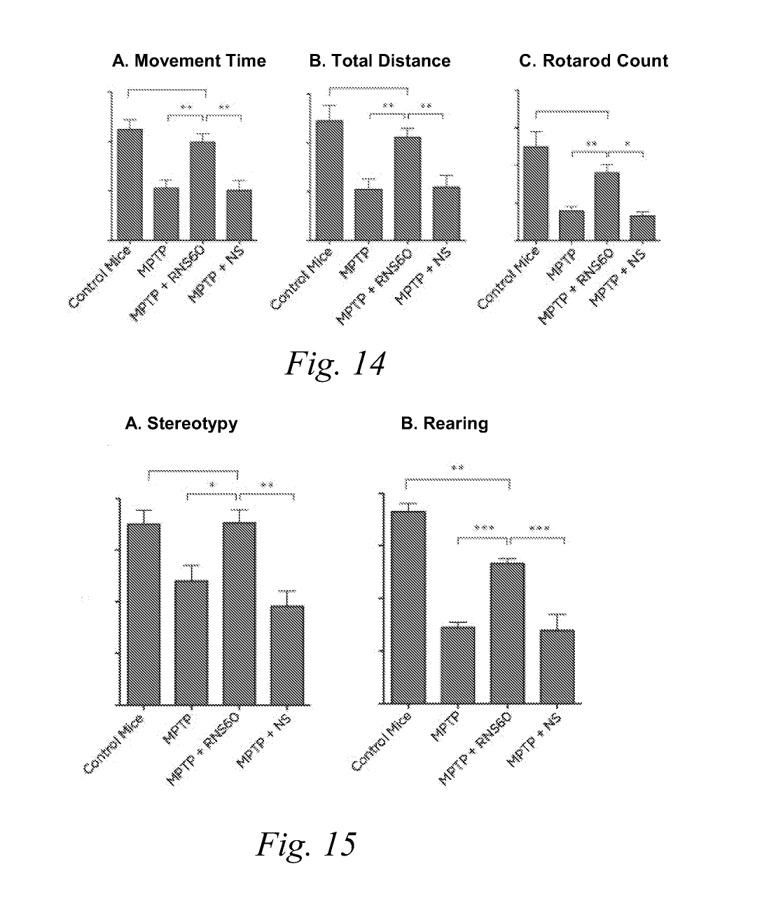

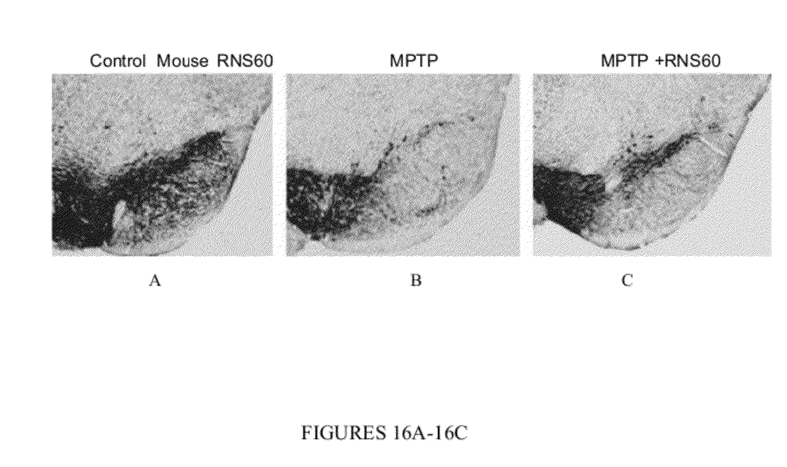

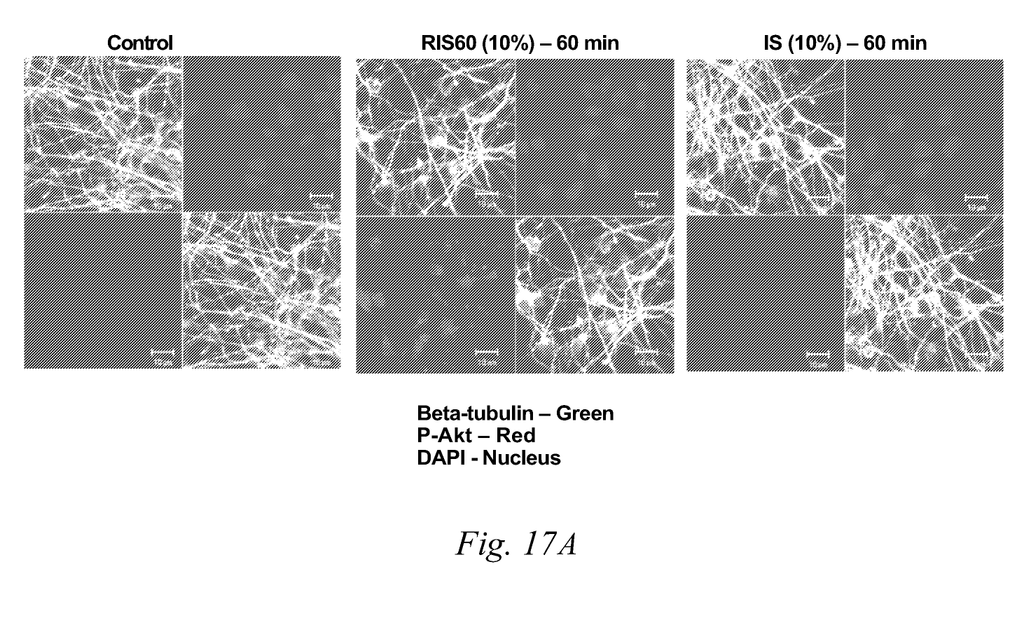

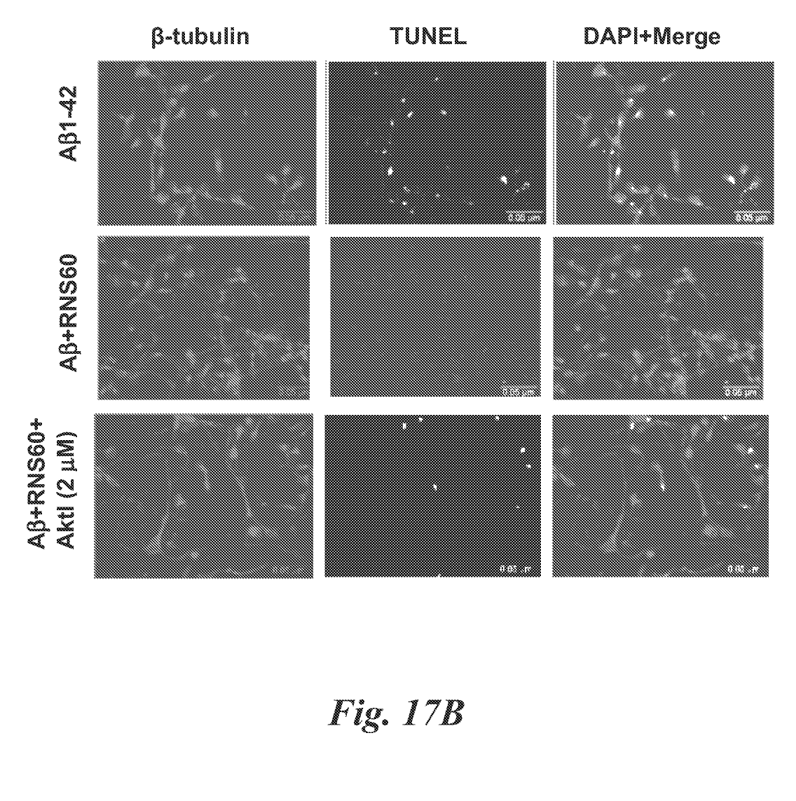

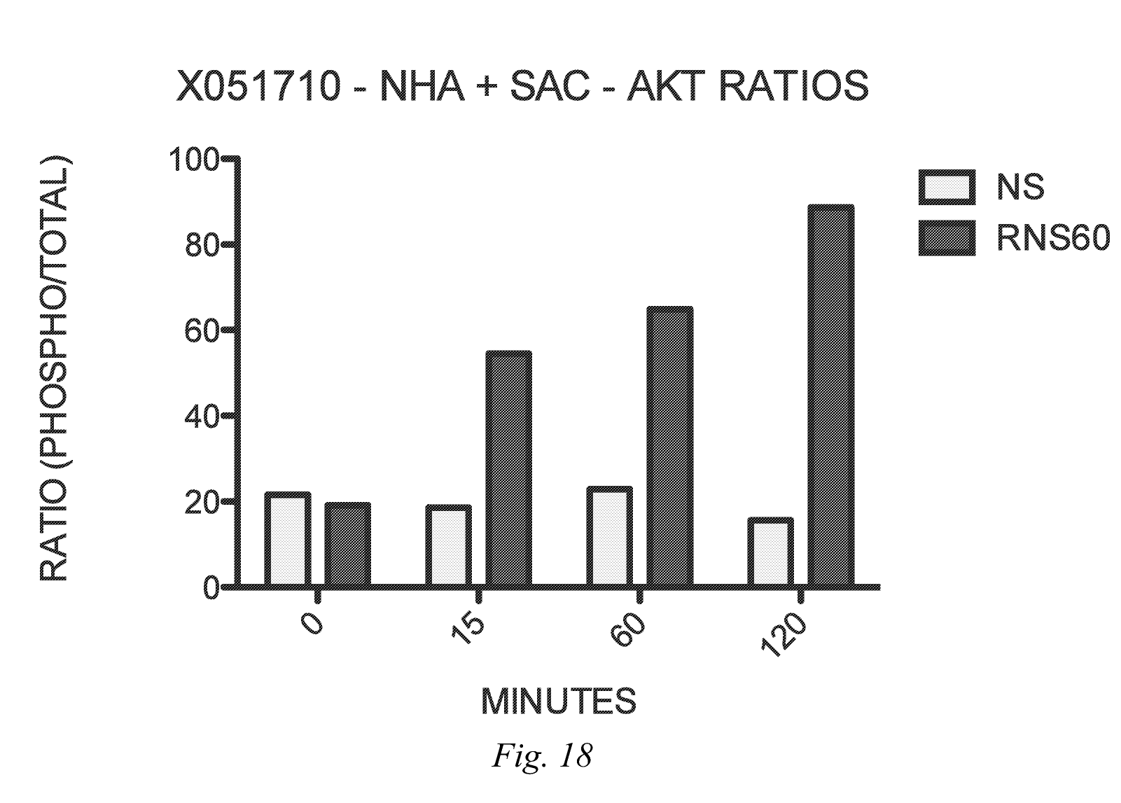

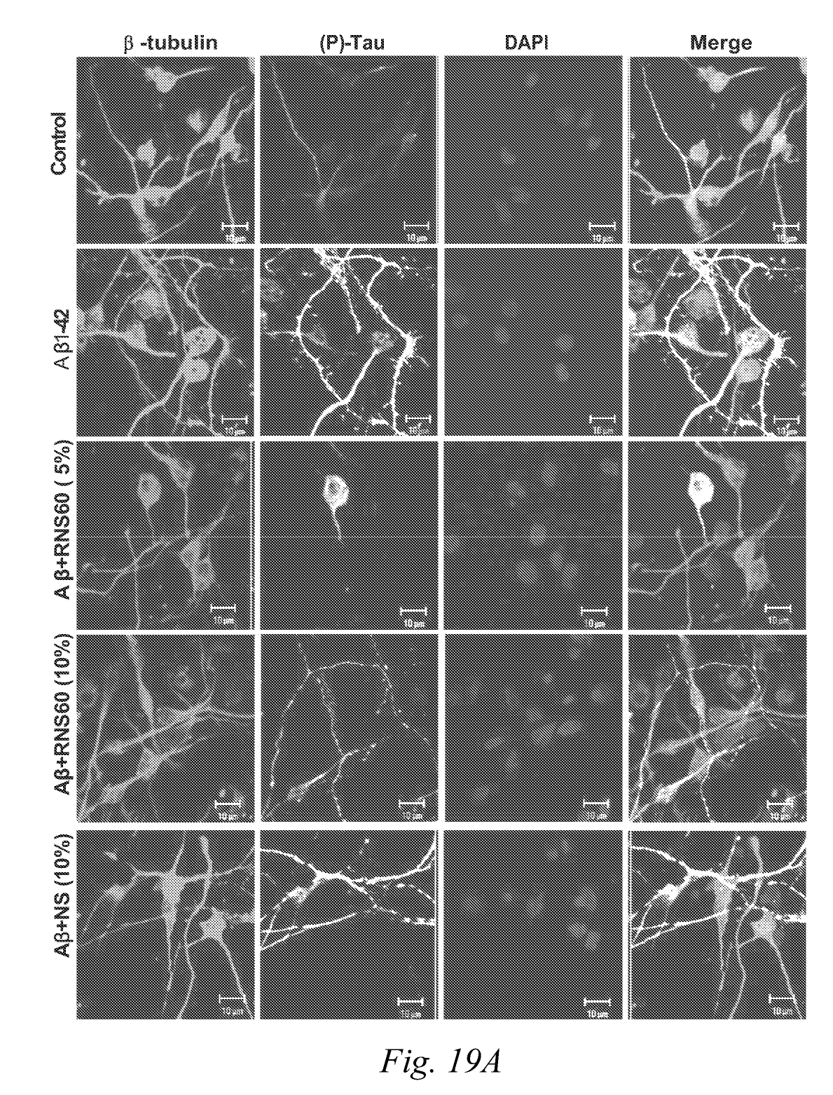

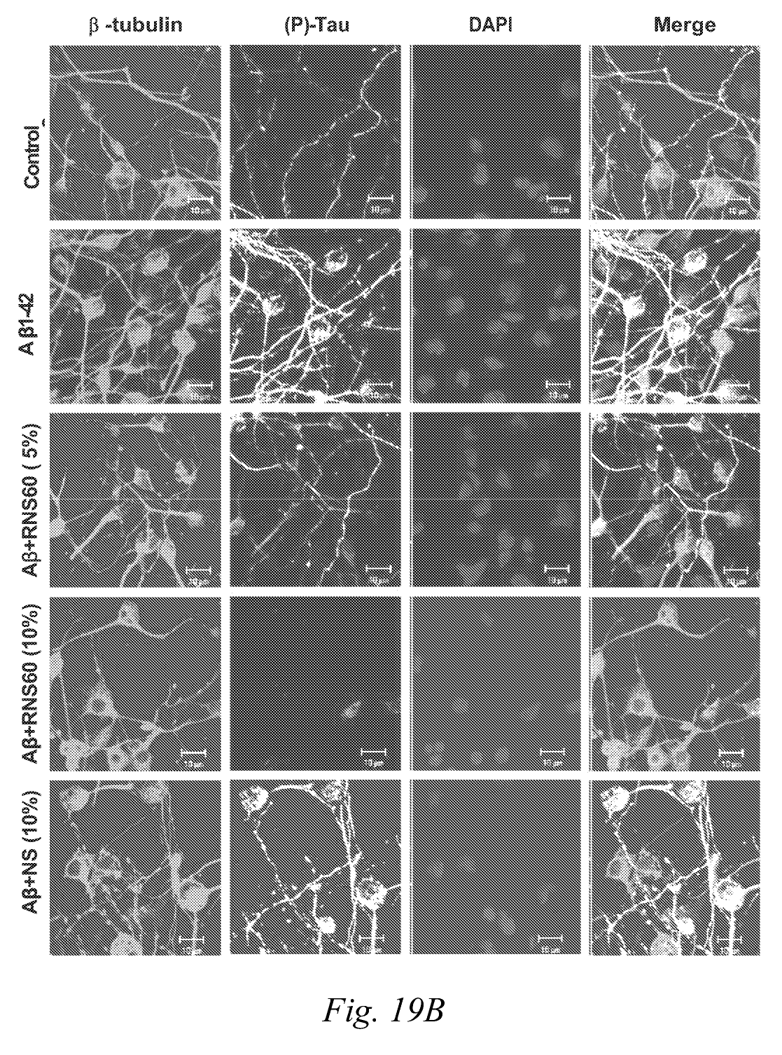

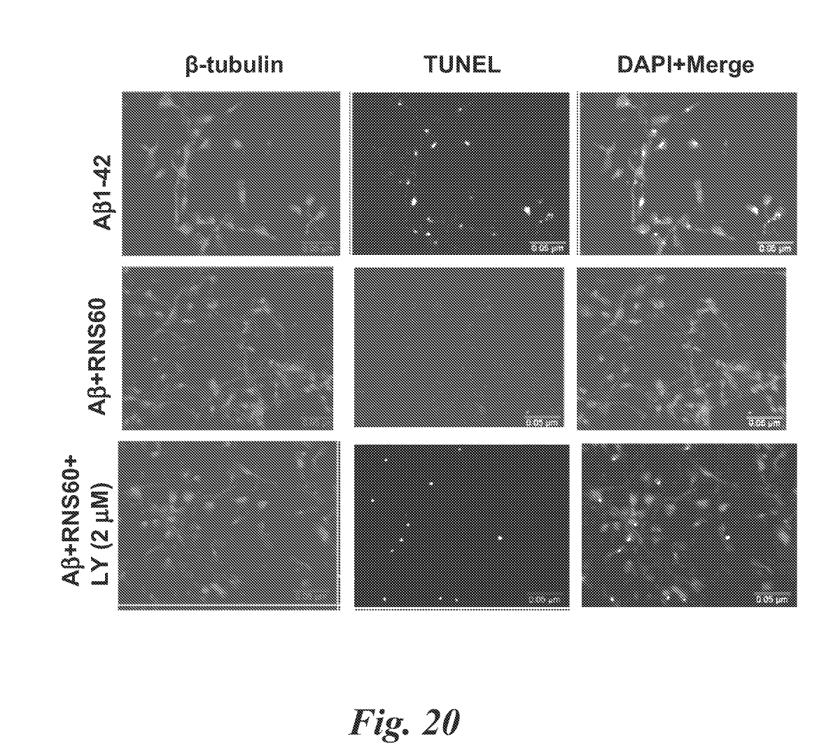

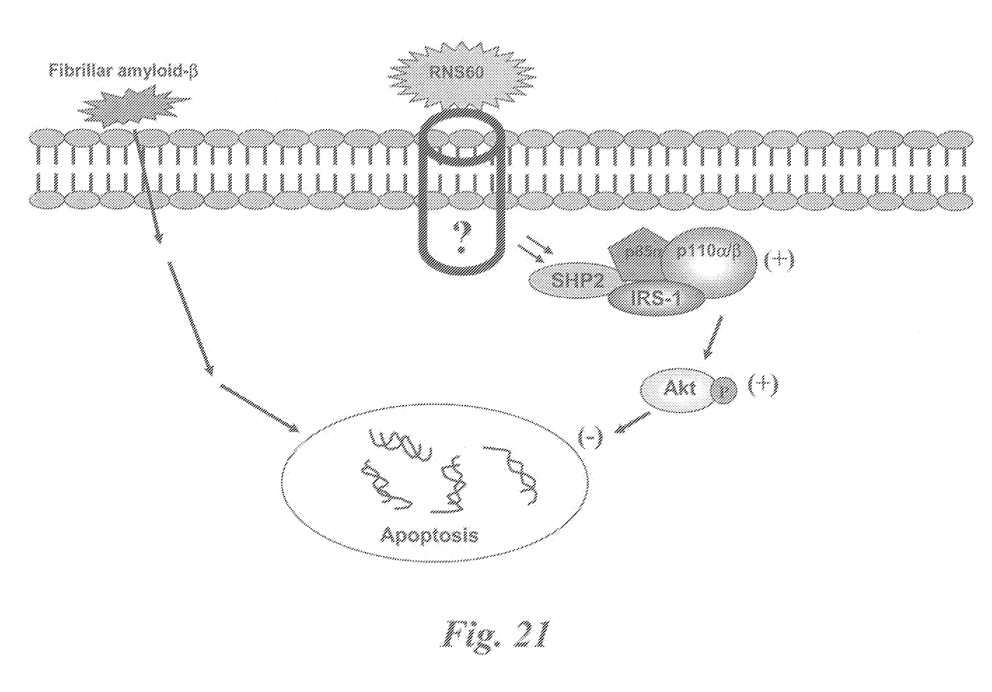

Experimental allergic encephalomyelitis (EAE) is a central nervous system (CNS) autoimmune demyelinating disease that mimics many of the clinical and pathologic features of multiple sclerosis (MS). The MOG murine model consists of a sensitization period, induced by the single subcutaneous (SC) injection of MOG emulsified in complete Freund's adjuvant (CFA) on study day 0 (200 μg MOG/300 μg CFA injected at a total dose volume of 200 μl/animal delivered as 2×100 μl subcutaneous bilateral injections over the paralumbar region); followed by intraperitoneal (IP) supplemental immunostimulation with pertussis toxin (PT) at 20 μg/kg (approximately 400 ng/mouse) via intraperitoneal (IP) injection once at the time of EAE induction on study day 0 and again, 48 hours later on study day 2 (Gilgun-Sherki Y. et al., Neurosciences Research 47:201-207, 2003). Animals were then treated with RNS60 IV infusion at indicated in FIG. 12. Animals used were Female C57BL/6J mice from Harlan Laboratories Israel, Ltd. (10 animals/group); young adults; 8-9 weeks old at study initiation.

All the animals were examined for signs of neurological responses and symptoms prior to EAE induction (study day 0) and thereafter examined on a daily basis throughout the 35-day observation period. EAE reactions were scored and recorded according to the art-recognized 0-15 scale in ascending order of severity. The clinical score was determined by summing the score of each section (see, e.g., Weaver et al., FASEB 2005; The FASEB Journal express article 10.1096/fj.04-2030fje. Published online Aug. 4, 2005.).

### Results:

FIG. 12 shows that RNS60, but not Vehicle control (Vehicle), is substantially efficacious in suppressing clinical score in a dose-responsive manner in an art-recognized mouse MOG model of Multiple Sclerosis(MS). Both high and low dose therapeutic daily administration of RNS-60, as well as the high dose administration of RNS-60 every three days (administration or RNS-60 in all instances beginning concomitant with first clinical signs), showed a marked decrease of clinical score (open diamonds=Vehicle control; open squares=dexamethasone positive control; light “x”s=low dose (0.09 ml RNS60) daily administration from onset of clinical signs; dark “x”s=high dose (0.2 ml RNS60) administration every three days from onset of clinical signs; and open triangles=high dose (0.2 ml RNS60) daily administration from onset of clinical signs).

In comparison with the MBP model of Example herein above, this mouse MOG model is known in the art for its ability to mimic the characteristic axonal damage of MS which the MBP model does not show, and extends the observed therapeutic efficacy over longer periods (28-30 days compared to 21 days with the MBP model). According to further aspects, RNS60, but not

Vehicle control (Vehicle), is substantially efficacious in reducing axonal damage in this mouse MOG model.

According to particular aspects of the present invention, the inventive electrokinetic compositions have substantial utility for treating, including alleviating and preventing, symptoms in an art-recognized mouse model of human MS. According to further aspects of the present invention, the inventive electrokinetic compositions have substantial utility for treating, including alleviating and preventing, the symptoms of MS in afflicted mammals (preferably humans).

In yet further aspects, the inventive electrokinetic compositions cross the Blood Brain Barrier (BBB), and thus provide a novel method for treating inflammatory conditions of the central nervous system.

### Example 10

### (RNS60, but not Normal Saline (NS), Attenuated the Activation of NFKB in MBP-primed T Cells)

Overview. NF-KB kinase is a kinase widely recognized in the art as mediating inflammatory responses in inflammation-mediated conditions and diseases.

This Example shows that RNS60, but not normal saline (NS), attenuated the activation of NFKB in MBP-primed T cells. According to particular aspects, therefore, the present electrokinetically-generated fluids have substantial utility for treating inflammation and inflammation-mediated conditions and diseases, including but not limited to, diabetes and related metabolic disorders, insulin resistance, neurodegenerative diseases (e.g., M.S., Parkinson's, Alzheimer's, etc), asthma, cystic fibrosis, vascular/coronary disease, retinal and/or macular degeneration, digestive disorders (e.g., inflammatory bowel disease, ulcerative colitis, Crohn's, etc.).

Methods. For the experiments shown in FIGS. 13A and 13B, T cells isolated from MBP-immunized mice were re-primed with MBP and after 24 h, cells received different concentrations of RNS60 and NS. After 2 h of treatment, DNA-binding activity of NF-KB was monitored in nuclear extracts by electrophoretic mobility shift assay (EMSA).

For experiments shown in FIG. 13C, T cells isolated from MBP-immunized mice were transfected with PBIIX-Luc, an NF-κB dependent reporter construct, followed by repriming with MBP. After 24 h of MBP priming, cells were treated with different concentrations of RNS60 and NS for 2 h followed by assay of luciferase activity in total cell extracts by a luciferase assay kit (Promega). In other cases, MBP-primed T cells were also stimulated with 30 nM PMA for 1 h. In these cases, PMA was added after 1 h of pretreatment with RNS60 and NS. Results are mean+SD of three different experiments.

Results. FIGS. 13A-C show that RNS60, but not normal saline (NS), attenuated the activation of NF-κB in MBP-primed T cells. Specifically, FIGS. 13A and 13B show that RNS60 (see middle three lanes of FIGS. 13A and 124B), but not NS (see right-most lane of FIGS. 13A and 13B), attenuated the activation of NF-κB in MBP-primed T cells in a dose-responsive manner.

Likewise, the bar graph of FIG. 13C shows that that RNS60 (see second, third and fourth bars of FIGS. 13A and 13B), but not NS (see fifth bar of FIGS. 13A and 13B), attenuated the activation of NF-κB in MBP-primed T cells, and hence also attenuated luciferase activity from the transfected NF-κB-dependent reporter construct (PBIIX-Luc) in total cell extracts, in a dose-responsive manner.

According to particular aspects, therefore, the disclosed electrokinetically-generated fluids have substantial utility for treating inflammation and inflammation-mediated conditions and diseases, including but not limited to, diabetes and related metabolic disorders, insulin resistance, neurodegenerative diseases (e.g., M.S., Parkinson's, Alzheimer's, etc), asthma, cystic fibrosis, vascular/coronary disease, retinal and/or macular degeneration, digestive disorders (e.g., inflammatory bowel disease, ulcerative colitis, Crohn's, etc.).

### Example 11

### (RNS60, but not Normal Saline (NS), Attenuated the MPTP Induced Pathological Signs of Parkinson's Disease in Mice)

### Overview:

Mice can be induced to exhibit pathological signs of Parkinson's disease (PD) (e.g., reduction in movement time, reduction in movement distance, lower ability to balance on a rotation rod, tremors, and loss of the striatum-controlled behavioral patterns stereotypy and rearing (vertical movements)) by treating them with 1-methyl-4-phenyl-1,2,3,6-tetrahydropyridine (MPTP). The neurotoxic effect of MPTP depends on its conversion into MPP⁺. In glial cells, monoamine oxidase B (MAO-B) converts MPTP to MPP⁺, which then activates glial cells, and recently, it has been shown that MPP⁺ induces the expression of proinflammatory molecules in microglia. In addition, MPP⁺ has been shown to cause apoptosis of dopaminergic neurons.

In this working EXAMPLE, the ability of RNS60 to reduce the pathological symptoms of PD (e.g., improve the coordinated movements, prevent the loss of straitum dependent behaviors, and rescue of dopaminergic neurons) in MPTP-treated mice was confirmed.

### Materials and Methods:

Briefly, C57BL/6 mice received four intraperitoneal injections of MPTP-HCl (18 mg/kg of free base) in saline at 2-hour intervals. Control animals received the same volume of saline. Treatment with RNS60 or normal saline (NS) started 1 day before the MPTP intoxication. Locomotor activity was measured 7 days after the MPTP injections with a computer-assisted Digiscan infrared activity monitor (FIGS. 14 and 15). Data are presented as mean±SEM, P values were calculated by ANOVA; *=P<0.05, **=P<0.01, ***=P<0.001, ns=not significant.

For the experiments verifying that RNS60 treatment rescues dopaminergic neurons in mice intoxicated with MPTP, the striatum was dissected 7 days after the MPTP intoxication detected by immunostaining with an antibody to tyrosine hydroxylase, the rate-limiting enzyme involved in dopamine synthesis. Panel A shows the striatum from the control mouse=healthy control mouse not intoxicated with MPTP, Panel B shows the striatum from the MPTP=MPTP-challenged mouse, Panel C shows the striatum from the MPTP+RNS60=MPTP-challenged mouse that was treated with RNS60.

### Results:

As evidenced by the locomotion analysis in FIGS. 14 and 15, MPP⁺ alone induced PD-like symptoms in the subjects, including reducing the movement time (FIG. 14A), distance (FIG. 14B), the ability of the mice to keep their balance on a rotating rod (FIG. 14C), loss of the striatum-controlled behavioral patterns stereotypy (grooming) (FIG. 15A), and rearing (vertical movements) (FIG. 15B). Signficantly, RNS60 substantially alleviated these symptoms and in some coordinated movement experiments the mice behavior was similar to the control mice. By contrast, under similar experimental conditions, mice pre-treated with the normal saline control (NS) and then induced with MPP⁺ had similar symptoms as MPP⁺ treatment alone (FIGS. 14 and 15). Thus, these data indicate that RNS60 had a specific protective effect on the MPP⁺-intoxicated mice.

Thus, FIGS. 14 and 15 show that the inventive electrokinetic fluid (RNS-60), but not control normal saline (NS), improves coordinated movements and prevents the loss of striatum-dependent behaviors of mice in a mouse model of PD.

Furthermore, immunostaining in the substantia nigra pars compacta, the part of the brain predominantly affected in PD, revealed a notable rescue of dopaminergic neurons in mice treated with RNS60 (FIG. 16), confirming the neuroprotective activity of the treatment. As can be seen in FIG. 16, MPP+intoxication led to the loss of tyrosine hydroxylase (TH)-positive neurons and the pre-treatment of RNS60 protected TH-positive neurons in the substantia nigra pars compacta (SNpc).

In addition, quantitation of striatal TH immunostaining of all groups of mice (n=6 per group) will be performed as described previously (1, 2). Optical density measurements will be obtained by digital image analysis (Scion). Striatal TH optical density basically reflects dopaminergic fiber innervation.

According to particular aspects therefore, because MPP⁺ is a neurotoxin, these results indicate that RNS60 has a protective effect from neurotoxins. According to further particular aspects, because MPP⁺ is a dopaminergic neurotoxin, these results indicate that RNS60 has a protective effect from dopaminergic neurotoxins.

According to particular aspects, the inventive electrokinetic fluids have substantial utility for preventing neurotoxic symptoms resulting from exposure to a neurotoxin.

### REFERENCES CITED IN THE ABOVE SECTION

1. Ghosh, A., Roy, A., Liu, X., Kordower, J. H., Mufson, E. J., Mosely, R. L., Ghosh, S., Gendelman, H. E. & Pahan, K. 2007. Selective inhibition of NF-κB activation prevents dopaminergic neuronal loss in a mouse model of Parkinson's disease. Proc. Natl. Acad. Sci. U.S.A. 104: 18754-18759.
2. Ghosh, A., Roy, A., Matras, J., Brahmachari, S., Gendelman, H. E., & Pahan, K. 2009. Simvastatin inhibits the activation of p21^{ras} and prevents the loss of dopaminergic neurons in a mouse model of Parkinson's disease. J. Neurosci. 29: 13543-13556.

### Example 12

(RNS60, but not Normal Saline (NS), Suppresses the MPTP Induced Expression of Microglial iNOS In Vivo in the Substantia Nigra Pars Compacta (SNpc))

### Overview:

According to particular aspects as described herein, the inventive electrokinetic fluids have substantial utility for protecting neural cells from neurotoxins.

Mice can be induced to exhibit pathological signs of Parkinson's disease (PD) by treating them with 1-methyl-4-phenyl-1,2,3,6-tetrahydropyridine (MPTP). The neurotoxic effect of MPTP depends on its conversion into MPP⁺. In glial cells, monoamine oxidase B (MAO-B) converts MPTP to MPP⁺, which then activates glial cells, and recently, it has been shown that MPP⁺ induces the expression of proinflammatory molecules in microglia. In addition, MPP⁺ causes apoptosis of dopaminergic neurons.

In the working EXAMPLES 7 and 11, the ability of RNS60 to inhibit MPP⁺-induced expression of inducible nitric oxide synthase (iNOS) and IL-1β in microglial cells and protect striatal dopaminergic neurons and improve locomotor activities in MPTP mouse model of PD was shown. Additional experiments are conducted to a) examine the effect of RIS60 on microglial iNOS in vivo in the substantia nigra pars compacta (SNpc) of MPTP-intoxicated mice.

### Materials and Methods:

Male C57BL/6 mice (n=3 in each group) receiving RIS60 or IS (300 μl/d/mouse via i.p. injection) one day prior to MPTP intoxication will receive four MPTP injections every 2 h interval. Treatment with RIS60/IS will continue and after 1 d of MPTP intoxication, mice are killed, and their brains are fixed, embedded, and processed for iNOS immunostaining as described previously (1,2). Briefly, ventral midbrain sections of all groups of mice (Saline, MPTP, MPTP-RIS60-300 μl, MPTP-IS-300 μl) undergo free-floating double-imunolabeling with antibodies against iNOS and CD11b (for microglia) as described (1-3).

CD11b-positive, iNOS-positive, and cells, which are positive for both CD11b and iNOS will be counted using the “Microsuite Biological Suite” software in Olympus IX81 fluorescent microscope to determine whether microglial activation and expression of iNOS is reduced in SNpc of RIS60-treated MPTP-intoxicated mice compared to that of control MPTP mice and IS

(Vehicle)-treated MPTP mice. Six nigral sections of each brain isolated from each of three animals are used to determine the effect of RIS60 on protein levels of CD11b and iNOS in the SNpc of MPTP-treated mice.

### Results:

According to certain embodiments, RNS60 but not normal saline suppresses the MPTP induced expression of microglial iNOS in vivo in the substantia nigra pars compacta (SNpc)). Thus these in vivo experiments confirm the results seen in Example 7, wherein semi-quantitative PCR showed that RNS60 but not normal saline suppresses the MPTP induced expression of iNOS in mouse microglial cells.

### REFERENCES CITED IN THE ABOVE SECTION

1. Ghosh, A., Roy, A., Liu, X., Kordower, J. H., Mufson, E. J. , Mosely, R. L., Ghosh, S., Gendelman, H. E. & Pahan, K. 2007. Selective inhibition of NF-κB activation prevents dopaminergic neuronal loss in a mouse model of Parkinson's disease. Proc. Natl. Acad. Sci. U.S.A. 104: 18754-18759.
2. Ghosh, A., Roy, A., Matras, J., Brahmachari, S., Gendelman, H. E., & Pahan, K. 2009. Simvastatin inhibits the activation of p21^{ras} and prevents the loss of dopaminergic neurons in a mouse model of Parkinson's disease. J. Neurosci. 29: 13543-13556.
3. Roy, A. & Pahan, K. 2010. Prospects of statins in Parkinson's disease. Neuroscientist 16: 000-000.

### Example 13

### (RNS60, but not Normal Saline (NS), Induced the Activation of Akt Phosphorylation in Primary Neurons and in Astrocytes)

Overview. Akt is a serine/threonine protein kinase that plays a key role in multiple cellular processes including glucose metabolism, cell proliferation, apoptosis, transcription and cell migration. Akt is known to regulate cellular survival. In particular, phosphorylated Akt has been shown to inhibit apoptosis by inactivating BAD (a pro-apoptoic protein). (See, Song G, et al., (2005). “The activation of Akt/PKB signaling pathway and cell survival”. J. Cell. Mol. Med. 9 (1): 59-71.) Phosphorylation of Akt, as recognized in the art, is a major player in protecting cells, including neural cells, from toxic and pro-apoptotic stimuli.

In this working EXAMPLE, the ability of RNS60 to induce phosphorylation of Akt in primary neural cells and astrocytes was confirmed. Further, the role of Akt in RNS60's ability to block apoptosis was demonstrated.

### Materials and Methods:

Neurons were isolated as described previously and cultured (1,2). Astrocytes were isolated and cultured as described previously. Neurons or astrocytes were treated with 10% RIS60 or IS (used as a control) for 0′, 15′, 30′, 60′, 90′, 120′, & 180′ monitored by western blot of cell extracts with antibodies against phospho-Akt and normal Akt (Cell Signaling). Total Akt was detected by antibodies against normal Akt. Neurons or astrocytes were treated with different doses RI560 (2%, 5%, 10%, & 20%). Different doses of IS were used as control. Activation of Akt was monitored as described above.

FIG. 17A shows the results from an experiment examining the effects of RNS60, compared with normal saline (NS) control, on inducing the phosphorylation of Akt in primary neurons. Akt phosphorylation was monitored by double-label immunofluorescence using antibodies against β-tubulin and phospho-Akt. Beta-tubulin was used as a marker for neurons and DAPI staining was used to visualize the nucleus of cells. Panels B and C shows that Akt phosphorylation was induced by 10% RNS60, whereas contol normal saline (“NS”) had no effect.

FIG. 17B shows the results from an experiment examining the effects of inhibiting Akt in primary neurons in the presence and absence of RNS60. Fibrillar Aβ1-42 (Bachem Biosciences) was changed into the fibrillar form as described previously (1,3). The function of phosphorylated Akt in neurons was inhibited by AktI (a specific inhibitor of Akt obtained from Calbiochem). Neurons preincubated with different concentrations of AktI for 30 min were treated with RNS60. After 1 h of incubation, cells were challenged with fibrillar Ab1-42. After 12 h, neuronal apoptosis was monitored by TUNEL and after 24 h, cell death was assessed by MTT and LDH release as described previously (1,2). The results (FIG. 17B) showed that the Akt inhibitor, AktI, abrogated the protective effect of RNS60 on fibrillar Ab-challenged neurons.

The results confirmed, therefore, that RNS60 requires Akt to protect neurons from Ab toxicity. FIG. 18 shows the results from a time course (0 minutes, 15 minutes, 60 minutes, and 120 minutes) experiment examining the effects of RNS60, compared with normal saline (NS) control, on inducing the phosphorylation of Akt in primary neurons. The graph represents the ratio between the amount of phosphorylated Akt to the total amount of Akt present in astrocytes when treated with either RNS60 or normal saline. As can be seen in FIG. 18, RNS60 induces a four-fold increase in Akt phosphorylation in astrocytes when compared to the effects of normal saline (NS). Thus, RNS60 specifically induces Akt phosphorylation.

According to particular aspects as described herein and not being bound by any particular mechanism, the inventive electrokinetic fluids have substantial utility for protecting neural cells from neurotoxins by preventing apoptosis induced by exposure to toxins.

### REFERENCES CITED IN THE ABOVE SECTION

1. Jana, A. & Pahan, K. 2004. Fibrillar amyloid-β peptides kill human primary neurons via NADPH oxidase-mediated activation of neutral sphingomyelinase: Implications for Alzheimer's disease. J. Biot Chem. 279: 51451-51459.
2. Jana, A. & Pahan, K. 2004. HIV-1 gp120 induces apoptosis in human primary neurons through redox-regulated activation of neutral sphingomyelinase. J. Neurosci. 24: 9531-9540.

### Example 14

### (RNS60, but Not Normal Saline (NS), Attenuated Fibrillar Aβ1-42 Peptide Induced Tau Phosphorylation in Primary Neurons)

Overview. Hyperphosphorylation of Tau is a hallmark of tangles in brain and neuronal tissue and can lead to one of several diseases which grouped together are known as taupathies. Taupathies include, but are not limited to Alzheimer's disease, argyorphilic grain disease, frontotemporal dementia, progressive supranuclear palsy, corticobasal degeneration, frontotemporal lobar degeneration (Pick's disease), and Dementia pugilistica (DP) (a.k.a., boxer's dementia, chronic boxer's encephalopathy).

FIGS. 19A-B show the results from an experiment examining the effects of RNS60 , compared with normal saline (NS) control, on fibrillar Aβ1(1-42)-mediated tau phosphorylation in primary neurons. Tau phosphorylation was monitored by double-label immunofluorescence using antibodies against β-tubulin and phospho-tau. Beta-tubulin was used as a marker for neurons and DAPI staining was used to visualize the nucleus of cells. The third and fourth panels from the top in the column labeled “(p)-Tau”, shows that Tau phosphorylation was inhibited by RNS60 in a dose-dependent manner, whereas contol normal saline (“NS”) had no effect, even at the high dose of 10% (see bottom panel the column labeled “(p)-Tau”.

### Example 15

### (The Protective Effect of RNS60 in the Presence of a Neurotoxin was Blocked by an Akt Inhibitor)

Overview. Akt is a serine/threonine protein kinase that plays a key role in multiple cellular processes including glucose metabolism, cell proliferation, apoptosis, transcription and cell migration. In particular, Akt is known to regulate cellular survival. In particular, phosphorylated Akt has bee shown to inhibit apoptosis by inactivating BAD (a pro-apoptoic protein). (See, Song G, et al., (2005). “The activation of Akt/PKB signaling pathway and cell survival”. J. Cell. Mol. Med. 9 (1): 59-71.) Phosphorylation of Akt, as recognized in the art, is a major player in protecting cells, including neural cells, from toxic and pro-apoptotic stimuli.

Working EXAMPLES 13 and 14 showed that a) Akt was phosphoylated in the presence of RNS60 in primary neurons and b) RNS60 attenuated fibrillar Aβ1-42 peptide induced Tau phosphorylation in primary neurons. The experiments disclosed in this working EXAMPLE confirmed that the protective effect of RNS60 in the presence of a neurotoxin could be blocked by an Akt inhibitor. Thus this EXAMPLE confirmed that RNS60 requires Akt to protect neurons from Aβ toxicity.

Neurons or astrocytes were isolated and cultured as described previously (1,2). Neurons or astrocytes were treated with 10% RIS60 or IS (used as a control) for 0′, 15′, 30′, 60′, 90′, 120′, & 180′ and activation of Akt was monitored by western blot of cell extracts with antibodies against phospho-Akt and normal Akt (Cell Signaling). Total Akt was detected by antibodies against normal Akt. Neurons or astrocytes were treated with increasing doses of RNS60 (2%, 5%, 10%, & 20%). Different doses of NS were used as control. Activation of Akt was monitored as described above.

Fibrillar Aβ1-42 (Bachem Biosciences) was changed into the fibrillar form as described previously (1,3). The function of phosphorylated Akt in neurons was inhibited by AktI (a specific inhibitor of Akt obtained from Calbiochem)

Neurons preincubated with different concentrations of AktI for 30 min were treated with RNS60. After 1 h of incubation, cells were challenged with fibrillar Aβ1-42. After 12 h, neuronal apoptosis was monitored by TUNEL and after 24 h, cell death was assessed by MTT and LDH release as described previously (1,2).

The results showed that the Akt inhibitor, AktI, abrogated the protective effect of RNS60 on fibrillar Aβ-challenged neurons. Thus, the results confirmed that RNS60 requires Akt to protect neurons from AP toxicity.

### REFERENCES CITED IN THE ABOVE SECTION

1. Jana, A. & Pahan, K. 2004. Fibrillar amyloid-β peptides kill human primary neurons via NADPH oxidase-mediated activation of neutral sphingomyelinase: Implications for Alzheimer's disease. J. Biol. Chem. 279: 51451-51459.
2. Jana, A. & Pahan, K. 2004. HIV-1 gp120 induces apoptosis in human primary neurons through redox-regulated activation of neutral sphingomyelinase. J. Neurosci. 24: 9531-9540.
3. Jana, M. & Pahan, K. 2008. Fibrillar amyloid-β peptides activate microglia via toll-like receptor 2: Implications for Alzheimer's disease. J. Immunol. 181: 7254-7262.

### Example 16

(The Protective Effect of RNS60 in the Presence of a Neurotoxin was blocked by a P1-3 kinase Inhibitor)

Overview. PI-3 kinase plays a key role in multiple cellular processes including cell growth, proliferation, differentiation, motility, survival and intracellular trafficking. In addition, PI 3-kinases are also a key component of the insulin signaling pathway. In particular, PI-3 kinase is known to phosphorylate, and hence, activate Akt, which is a major player in protecting cells, including neural cells, from toxic and pro-apoptotic stimuli.

Working EXAMPLES 13 and 15 herein above showed that: a) Akt was phosphoylated in the presence of RNS60 in primary neurons; and b) RNS60-mediated protection from a neurotoxin could be blocked by an Akt inhibitor. This EXAMPLE further demonstrates that RNS60-mediated protection from neurotoxin induced apoptosis requires the PI-3 kinase pathway.

FIG. 20 shows the results from an experiment examining the effects of RNS60 on human primary neurons that have been treated with a PI-3 kinase inhibitor. Human primary neurons were isolated and cultured as described previously (1,2). Fibrillar A131-42 (Bachem Biosciences) was changed into the fibrillar form as described previously (1,3). The function of PI-3 kinase in neurons was inhibited by LY294002 (a specific inhibitor of PI-3 kinase obtained from Enogene).

Neurons preincubated with 2 μm LY294002 were treated with RNS60. After 1 h of incubation, cells were challenged with fibrillar Aβ1-42. After 12 h, neuronal apoptosis was monitored by TUNEL and after 24 h, cell death was assessed by MTT and LDH release as described previously (1,2).

The results showed that the PI-3 kinase inhibitor, LY294002, abrogated the protective effect of RNS60 on fibrillar AP-challenged neurons. Thus, the results demonstrate that RNS60 requires PI-3 kinase to protect neurons from AP toxicity.

According to certain embodiments, therefore, and as schematically represented in FIG. 21, in neurons, RNS60 activates PI-3 kinase via membrane effects (e.g., via modulation of ion channel(s), which in turn phosphorylates and activates Akt. Phosphorylated Akt then blocks neurotoxin-mediated apoptosis of the neuronal cells.

Incorporation by Reference. All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

It should be understood that the drawings and detailed description herein are to be regarded in an illustrative rather than a restrictive manner, and are not intended to limit the invention to the particular forms and examples disclosed. On the contrary, the invention includes any further modifications, changes, rearrangements, substitutions, alternatives, design choices, and embodiments apparent to those of ordinary skill in the art, without departing from the spirit and scope of this invention, as defined by the following claims. Thus, it is intended that the following claims be interpreted to embrace all such further modifications, changes, rearrangements, substitutions, alternatives, design choices, and embodiments.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Accordingly, the invention is not limited except as by the appended claims.

1. A method for protecting against or reducing neurotoxicity of exposure to a neurotoxic agent, comprising administering to a subject in need thereof a therapeutically effective amount of an electrokinetically altered aqueous fluid comprising an ionic aqueous solution of charge-stabilized oxygen-containing nanostructures substantially having an average diameter of less than about 100 nanometers and stably configured in the ionic aqueous fluid in an amount sufficient to provide for neuroprotection against the neurotoxic agent, wherein an method for protecting against or reducing neurotoxicity of exposure to a neurotoxic agent is afforded.

2. The method of claim 1, comprising protecting against or reducing loss of motor coordination in the subject exposed to the neurotoxin.

3. The method of claim 1, wherein protecting or reducing neurotoxin-mediated neuronal apoptosis is afforded.

4. The method of claim 1, comprising activating or inducing at least one of PI-3 kinase and Akt phosphorylation in neurons of the subject.

5. The method of claim 1, wherein the charge-stabilized oxygen-containing nanostructures are stably configured in the ionic aqueous fluid in an amount sufficient to provide, upon contact of a living cell by the fluid, modulation of at least one of cellular membrane potential and cellular membrane conductivity.

6. The method of any one of claims 1 through 5, wherein administering the fluid comprises administering the fluid prior to exposure to the neurotoxic agent.

7. The electrokinetic fluid of claim 1, wherein the charge-stabilized oxygen-containing nanostructures are the major charge-stabilized gas-containing nanostructure species in the fluid.

8. The electrokinetic fluid of claim 1, wherein the percentage of dissolved oxygen molecules present in the fluid as the charge-stabilized oxygen-containing nanostructures is a percentage selected from the group consisting of greater than: 0.01%, 0.1%, 1%, 5%; 10%; 15%; 20%; 25%; 30%; 35%; 40%; 45%; 50%; 55%; 60%; 65%; 70%; 75%; 80%; 85%; 90%; and 95%.

9. The electrokinetic fluid of claim 1, wherein the total dissolved oxygen is substantially present in the charge-stabilized oxygen-containing nanostructures.

10. The electrokinetic fluid of claim 1, wherein the charge-stabilized oxygen-containing nanostructures substantially have an average diameter of less than a size selected from the group consisting of: 90 nm; 80 nm; 70 nm; 60 nm; 50 nm; 40 nm; 30 nm; 20 nm; 10 nm; and less than 5 nm.

11. The electrokinetic fluid of claim 1, wherein the ionic aqueous solution comprises a saline solution.

12. The electrokinetic fluid of claim 1, wherein the fluid is superoxygenated.

13. The electrokinetic fluid of claim 1, wherein the fluid comprises a form of solvated electrons.

14. The method of claim 1, wherein alteration of the electrokinetically altered aqueous fluid comprises exposure of the fluid to hydrodynamically-induced, localized electrokinetic effects.

15. The method of claim 14, wherein, exposure to the localized electrokinetic effects comprises exposure to at least one of voltage pulses and current pulses.

16. The method of claim 14, wherein the exposure of the fluid to hydrodynamically-induced, localized electrokinetic effects, comprises exposure of the fluid to electrokinetic effect-inducing structural features of a device used to generate the fluid.

17. The method of claim 1, wherein the electrokinetically altered aqueous fluid modulates localized or cellular levels of nitric oxide.

18. The method of claim 1 wherein the electrokinetically altered aqueous fluid promotes a localized decrease at the site of administration of at least one cytokine selected from the group consisting of: IL-1beta, IL-8, TNF-alpha, and TNF-beta.

19. The method of claim 1, further comprising combination therapy, wherein at least one additional therapeutic agent is administered to the patient.

20. The method of claim 19, wherein, the at least one additional therapeutic agent is selected from the group consisting of: adrenergic neurotoxins, cholinergic neurotoxins, dopaminergic neurotoxins, excitotoxins and chemotherapeutic agents.

21. The method of claim 5, wherein modulation of at least one of cellular membrane potential and cellular membrane conductivity comprises modulating at least one of cellular membrane structure or function comprising modulation of at least one of a conformation, ligand binding activity, or a catalytic activity of a membrane associated protein.

22. The method of claim 21, wherein the membrane associated protein comprises at least one selected from the group consisting of receptors, transmembrane receptors, ion channel proteins, intracellular attachment proteins, cellular adhesion proteins, and integrins.

23. The method of claim 22, wherein the transmembrane receptor comprises a G-Protein Coupled Receptor (GPCR).

24. The method of claim 23, wherein the G-Protein Coupled Receptor (GPCR) interacts with a G protein a subunit.

25. The method of claim 24, wherein the G protein a subunit comprises at least one selected from the group consisting of Gαₛ, Gαᵢ, Gα_{q}, and Gα₁₂.

26. The method of claim 25, wherein the at least one G protein a subunit is Ga_{q}.

27. The method of claim 5, wherein modulating cellular membrane conductivity, comprises modulating whole-cell conductance.

28. The method of claim 27, wherein modulating whole-cell conductance, comprises modulating at least one voltage-dependent contribution of the whole-cell conductance.

29. The method of claim 5, wherein modulation of at least one of cellular membrane potential and cellular membrane conductivity comprises modulating intracellular signal transduction comprising modulation of a calcium dependant cellular messaging pathway or system.

30. The method of claim 5, wherein modulation of at least one of cellular membrane potential and cellular membrane conductivity comprises modulating intracellular signal transduction comprising modulation of phospholipase C activity.

31. The method of claim 5, wherein modulation of at least one of cellular membrane potential and cellular membrane conductivity comprises modulating intracellular signal transduction comprising modulation of adenylate cyclase (AC) activity.

32. The method of claim 5, wherein modulation of at least one of cellular membrane potential and cellular membrane conductivity comprises modulating intracellular signal

33. The method of claim 1, comprising administration to a cell network or layer, and further comprising modulation of an intercellular junction therein.

34. The method of claim 33, wherein the intracellular junction comprises at least one selected from the group consisting of tight junctions, gap junctions, zona adherins and desmasomes.

35. The method of claim 33, wherein the cell network or layers comprises at least one selected from the group consisting of endothelial cell and endothelial-astrocyte tight junctions in CNS vessels, blood-cerebrospinal fluid tight junctions or barrier, pulmonary epithelium-type junctions, bronchial epithelium-type junctions, and intestinal epithelium-type junctions.

36. The method of claim 1, wherein the electrokinetically altered aqueous fluid is oxygenated, and wherein the oxygen in the fluid is present in an amount of at least 8 ppm, at least 15, ppm, at least 25 ppm, at least 30 ppm, at least 40 ppm, at least 50 ppm, or at least 60 ppm oxygen at atmospheric pressure.

37. The method of claim 1, wherein the amount of oxygen present in charge-stabilized oxygen-containing nanostructures of the electrokinetically-altered fluid is at least 8 ppm, at least 15, ppm, at least 20 ppm, at least 25 ppm, at least 30 ppm, at least 40 ppm, at least 50 ppm, or at least 60 ppm oxygen at atmospheric pressure.

38. The method of claim 1, wherein the electrokinetically altered aqueous fluid comprises at least one of a form of solvated electrons, and electrokinetically modified or charged oxygen species.

39. The method of claim 38, wherein the form of solvated electrons or electrokinetically modified or charged oxygen species are present in an amount of at least 0.01 ppm, at least 0.1 ppm, at least 0.5 ppm, at least 1 ppm, at least 3 ppm, at least 5 ppm, at least 7 ppm, at least 10 ppm, at least 15 ppm, or at least 20 ppm.

40. The method of claim 38, wherein the electrokinetically altered oxygenated aqueous fluid comprises solvated electrons stabilized, at least in part, by molecular oxygen.

41. The method of claim 5, wherein the ability to modulate of at least one of cellular membrane potential and cellular membrane conductivity persists for at least two, at least three, at least four, at least five, at least 6, at least 12 months, or longer periods, in a closed gas-tight container.

42. The method of claim 21, wherein the membrane associated protein comprises CCR3.

43. The method of claim 1, wherein treating comprises administration by at least one of topical, inhalation, intranasal, oral and intravenous.

44. The method of claim 1, wherein , the charge-stabilized oxygen-containing nanostructures of the electrokinetically-alterd fluid comprise at least one salt or ion from Tables 1 and 2 disclosed herein.

45. A pharmaceutical composition, comprising an amount of an electrokinetically altered aqueous fluid comprising an ionic aqueous solution of charge-stabilized oxygen-containing nanostructures substantially having an average diameter of less than about 100 nanometers and stably configured in the ionic aqueous fluid in an amount sufficient for protecting against or reducing neurotoxicity of exposure to a neurotoxic agent.

46. A method for preserving or improving motor coordination in a subject, having a neurodegenerative condition or disease, comprising administering to a subject having a neurodegenerative condition or disease characterized by loss of motor coordination, a therapeutically effective amount of an electrokinetically altered aqueous fluid comprising an ionic aqueous solution of charge-stabilized oxygen-containing nanostructures substantially having an average diameter of less than about 100 nanometers and stably configured in the ionic aqueous fluid in an amount sufficient to provide for preserving or improving motor coordination in the subject, wherein a method for preserving or improving motor coordination in a subject having a neurodegenerative condition or disease is afforded.

47. The method of claim 46, comprising activation or induction of at least one of P1-3 kinase and Akt phosphorylation.

48. The method of claim 46, wherein the neurodegenerative condition or disease comprises at least one inflammatory neurrodegenerative condition or disease selected from the group consisting of multiple sclerosis, amyotrophic lateral sclerosis, Alzheimer's disease,

49. The method of claim 48, wherein the inflammatory neurodegenerative condition or disease comprises at least one of multiple sclerosis, amyotrophic lateral sclerosis, Alzheimer's disease, Parkinson's disease.

50. The method of claim 46, further comprising a synergistic or non-synergistic inhibition or reduction in inflammation by simultaneously or adjunctively treating the subject with another anti-inflammatory agent.

51. The method of claim 50, wherein said other anti-inflammatory agent comprises a steroid or glucocorticoid steroid.

52. The method of claim 51, wherein the glucocorticoid steroid comprises Budesonide or an active derivative thereof.

53. The method of claim 46, further comprising combination therapy, wherein at least one additional therapeutic agent is administered to the patient.

54. The method of claim 53, wherein, the at least one additional therapeutic agent is selected from the group consisting of: glatiramer acetate, interferon-β, mitoxantrone, natalizumab, inhibitors of MMPs including inhibitor of MMP-9 and MMP-2, short-acting β₂-agonists, long-acting β₂-agonists, anticholinergics, corticosteroids, systemic corticosteroids, mast cell stabilizers, leukotriene modifiers, methylxanthines, β₂-agonists, albuterol, levalbuterol, pirbuterol, artformoterol, formoterol, salmeterol, anticholinergics including ipratropium and tiotropium; corticosteroids including beclomethasone, budesonide, flunisolide, fluticasone, mometasone, triamcinolone, methyprednisolone, prednisolone, prednisone; leukotriene modifiers including montelukast, zatirhikast, and zileuton; mast cell stabilizers including cromolyn and nedocromil; methylxanthines including theophylline; combination drugs including ipratropium and albuterol, fluticasone and salmeterol, budesonide and formoterol; antihistamines including hydroxyzine; diphenhydramine, loratadine, cetirizine, and hydrocortisone; immune system modulating drugs including tacrolimus and pimecrolimus; cyclosporine; azathioprine; mycophenolatemofetil and combinations thereof.

55. The method of claim 53, wherein the at least one additional therapeutic agent is a TSLP and/or TSLPR antagonist.

56. The method of claim 55, wherein the TSLP and/or TSLPR antagonist is selected from the group consisting of neutralizing antibodies specific for TSLP and the TSLP receptor, soluble TSLP receptor molecules, and TSLP receptor fusion proteins, including TSLPR-immunoglobulin Fc molecules or polypeptides that encode components of more than one receptor chain.
